(19) United States
(12) Patent Application Publication
(10) Pub. No.: US 2006/0051373 A1
(43) Pub. Date: Mar. 9, 2006

(54) Particle-bound human immunodeficiency virus envelope glycoproteins and related compositions and methods

(76) Inventors: William C. Olson, Ossining, NY (US); Norbert Schulke, New City, NY (US); Jason Gardner, Ardsley, NY (US); Paul J. Maddon, Scarsdale, NY (US)

Correspondence Address: COOPER & DUNHAM, LLP, 1185 AVENUE OF THE AMERICAS, NEW YORK, NY, 10036, US

(21) Appl. No.: 10/510,268

(22) PCT Filed: Sep. 6, 2002
(86) PCT No.: PCT/US02/28332

§ 371 Date: Jul. 11, 2005

Related U.S. Application Data
(60) Provisional application No. 60/370,410, filed on Apr. 5, 2002.

(51) Int. Cl. A61K 39/21; A61K 39/12; A61K 38/20; A61K 38/19; A61K 31/739

(52) U.S. Cl. 424/204.1; 424/208.1; 424/85.1; 514/54; 424/85.2

(57) ABSTRACT

This invention provides a first composition comprising a pharmaceutically acceptable particle and a stable HIV-1 pre-fusion envelope glycoprotein trimeric complex operably affixed thereto. This invention further provides a second composition comprising (a) a pharmaceutically acceptable particle, (b) an antigen, and (c) an agent which is operably affixed to the particle and is specifically bound to the antigen, whereby the antigen is operably bound to the particle. Finally, this invention provides related nucleic acids, vectors, cells, compositions, production methods, and prophylactic and therapeutic methods.

The invention disclosed herein was made with government support under. NIH Grant Nos. R01 AI39420, R01 AI42382, R01 AI45463, R21 AI44291, R21 AI49566, and U01 AI49764 from the Department of Health and Human Services. Accordingly, the government has certain rights in this invention.

Throughout this application, various publications are referenced. The disclosures of these publications are hereby incorporated by reference into this application to describe more fully the art to which this invention pertains.

### BACKGROUND OF THE INVENTION

I. Viral Envelope Glycoproteins

The human immunodeficiency virus (HIV) is the agent that causes Acquired Immunodeficiency Syndrome (AIDS), a lethal disease characterized by deterioration of the immune system. The initial phase of the HIV replicative cycle involves the attachment of the virus to susceptible host cells followed by fusion of viral and cellular membranes.

These events are mediated by the exterior viral envelope glycoproteins, which are first synthesized as a fusion-incompetent precursor envelope glycoprotein (env) known as gp160. The gp160 glycoprotein is endoproteolytically processed to the mature envelope glycoproteins gp120 and gp41, which are noncovalently associated with each other in a complex on the surface of the virus. The gp120 surface protein contains the high affinity binding site for human CD4, the primary receptor for HIV, as well as domains that interact with fusion coreceptors, such as the chemokine receptors CCR5 and CXCR4. The gp41 protein spans the viral membrane and contains at its amino-terminus a sequence of amino acids important for the fusion of viral and cellular membranes.

The native, fusion-competent form of the HIV-1 envelope glycoprotein complex is a trimeric structure composed of three gp120 and three gp41 subunits. The receptor-binding (CD4 and co-receptor) sites are located in the gp120 moieties, and the fusion peptides in the gp41 components (Chan, 1997; Kwong, 1998; Kwong, 2000; Poignard, 2001; Tan, 1997; Weissenhorn, 1997; and Wyatt, 1998a).

In the generally accepted model of HIV-1 fusion, the sequential binding of gp120 to CD4 and a co-receptor induces a series of conformational changes in the gp41 subunits, leading to the insertion of the fusion peptides into the host cell membrane in a highly dynamic process (Doms, 2000; Jones, 1998; Melikyan, 2000; Sattentau, 1991; Sullivan, 1998; Trkola, 1996; Wu, 1996; Wyatt, 1998b; and Zhang, 1999). The associations between the six components of the fusion-competent complex are maintained via non-covalent interactions between gp120 and gp41, and between the gp41 subunits (Poignard, 2001; and Wyatt, 1998b). These interactions are relatively weak, making the fusion-competent complex unstable. This instability perhaps facilitates the conformational changes in the various components that are necessary for the fusion reaction to proceed efficiently, but it greatly complicates the task of isolating the native complex in purified form. Put simply, the native complex falls apart before it can be purified, leaving only the dissociated subunits.

Because of their location on the virion surface and central role in mediating viral entry, the HIV envelope glycoproteins provide important targets for HIV vaccine development. Although most HIV-infected individuals mount a robust antibody (Ab) response to the envelope glycoproteins, most anti-gp120 and anti-gp41 antibodies produced during natural infection bind weakly or not at all to virions and are thus functionally ineffective. These antibodies are probably elicited and affinity matured against “viral debris” comprising gp120 monomers or improperly processed oligomers released from virions or infected cells. (Burton, 1997).

Several preventive HIV-1 subunit vaccines have been tested in Phase I and II clinical trials and a multivalent formulation is entering Phase III testing. These vaccines have contained either monomeric gp120 or unprocessed gp160 proteins. In addition, the vaccines mostly have been derived from viruses adapted to grow to high levels in immortalized T cell lines (TCLA viruses). These vaccines have consistently elicited antibodies which neutralize the homologous strain of virus and some additional TCLA viruses.

However, the antibodies do not potently neutralize primary HIV-1 isolates (Mascola, 1996). Compared with TCLA strains, the more clinically relevant primary isolates typically possess a different cellular tropism, show a different pattern of coreceptor usage, and have reduced sensitivity to neutralization by soluble CD4 and antibodies. These differences primarily map to the viral envelope glycoproteins (Moore, 1995).

The Importance of Oligomerization in Envelope Glycoprotein Structure

There is a growing awareness that current-generation HIV subunit vaccines do not adequately present key neutralization epitopes as they appear on virions (Parren, 1997). There are several ways in which the native structure of virions affects the presentation of antibody epitopes. First, much of the surface area of gp120 and gp41 is occluded by inter-subunit interactions within the trimer. Hence several regions of gp120, especially around the N- and C-termini, that are well exposed (and highly immunogenic) on the monomeric form of the protein, are completely inaccessible on the native trimer (Moore, 1994a). This means that a subset of antibodies raised to gp120 monomers are irrelevant, whether they arise during natural infection (because of the shedding of gp120 monomers from virions or infected cells) or after gp120 subunit vaccination. This provides yet another level of protection for the virus; the immune system is decoyed into making antibodies to shed gp120 that are poorly reactive, and hence ineffective, with virions.

A second, more subtle problem is that the structure of key gp120 epitopes can be affected by oligomerization. A classic example is provided by the epitope for the broadly neutralizing human MAb IgG1b12 (Burton, 1994). This epitope overlaps the CD4-binding site on gp120 and is present on monomeric gp120. However, IgG1b12 reacts far better with native, oligomeric gp120 than might be predicted from its monomer reactivity, which accounts for its unusually potent neutralization activity. Thus, the IgG1b12 epitope is oligomer-dependent, but not oligomer-specific.

The converse situation is more common, unfortunately. Many antibodies that are strongly reactive with CD4-binding site-related epitopes on monomeric gp120 fail to react with the native trimer, and consequently do not neutralize the virus. In some undefined way, oligomerization of gp120 adversely affects the structures recognized by these monoclonal antibodies (Mabs)(Fouts, 1997).

A third example of the problems caused by the native structure of the HIV-1 envelope glycoproteins is provided by gp41 MAbs. Only a single gp41 MAb (2F5) is known to have strong neutralizing activity against primary viruses (Trkola, 1995), and among those tested, 2F5 alone is thought to recognize an intact, gp120-gp41 complex (Sattentau, 1995). All other gp41 MAbs that bind to virions or virus-infected cells probably react with fusion-incompetent gp41 structures from which gp120 has dissociated. Since the most stable form of gp41 is this post-fusion configuration (Weissenhorn, 1997), it can be supposed that most anti-gp41 antibodies are raised (during natural infection or after gp160 vaccination) to an irrelevant gp41 structure that is not present on the pre-fusion form.

Despite these protective mechanisms, most HIV-1 isolates are potently neutralized by a limited subset of broadly reactive human MAbs, so induction of a relevant humoral immune response is not impossible. Mab IgG1b12 blocks gp120-CD4 binding; a second (2G12; Trkola, 1996) acts mostly by steric hindrance of virus-cell attachment; and 2F5 acts by directly compromising the fusion reaction itself. Critical to understanding the neutralization capacity of these MAbs is the recognition that they react preferentially with the fusion-competent, oligomeric forms of the envelope glycoproteins, as found on the surfaces of virions and virus-infected cells (Parren, 1998). This distinguishes them from their less active peers. The limited number of MAbs that are oligomer-reactive explains why so few can neutralize primary viruses. Thus, with rare exceptions, neutralizing anti-HIV antibodies are capable of binding infectious virus while non-neutralizing antibodies are not (Fouts, 1998) Neutralizing antibodies also have the potential to clear infectious virus through effector functions, such as complement-mediated virolysis.

Modifying the Antigenic Structure of the HIV Envelope Glycoproteins

HIV-1 has evolved sophisticated mechanisms to shield key neutralization sites from the humoral immune response, and in principle these mechanisms can be “disabled” in a vaccine. One example is the V3 loop, which for TCLA viruses in particular is an immunodominant epitope that directs the antibody response away from more broadly conserved neutralization epitopes. HIV-1 is also protected from humoral immunity by the extensive glycosylation of gp120.

When glycosylation sites were deleted from the V1/V2 loops of SIV gp120, not only was a neutralization-sensitive virus created, but the immunogenicity of the mutant virus was increased so that a better immune response was raised to the wild-type virus (Reitter, 1998). Similarly, removing the V1/V2 loops from HIV-1 gp120 renders the conserved regions underneath more vulnerable to antibodies (Cao, 1997), although it is not yet known whether this will translate into improved immunogenicity.

Of note is that the deletion of the V1, V2 and V3 loops of the envelope glycoproteins of a TCLA virus did not improve the induction of neutralizing antibodies in the context of a DNA vaccine (Lu, 1998). However, the instability of the gp120-gp41 interaction, perhaps exacerbated by variable loop deletions, may have influenced the outcome of this experiment. By increasing the time that the gp120-gp41 complex is presented to the immune system, stabilized envelope proteins expressed in vivo provide a means in principle to significantly improve upon the immune response elicited during natural infection.

Native and Non-Native Oligomeric Forms of the HIV Envelope Glycoproteins

Current data suggest that on the HIV virion three gp120 moieties are non-covalently associated with three, underlying gp41 components in a meta-stable configuration whose fusion potential is triggered by interaction with cell surface receptors. This pre-fusion form may optimally present neutralization epitopes. We refer to this form of the envelope glycoproteins as native gp120-gp41. However, other oligomeric forms are possible, and these are defined in FIG. 1.

gp160: The full-length gp160 molecule often aggregates when expressed as a recombinant protein, at least in part because it contains the hydrophobic transmembrane domain. One such molecule is derived from a natural mutation that prevents the processing of the gp160 precursor to gp120/gp41 (VanCott, 1997). The gp160 precursor does not mediate virus-cell fusion and is a poor mimic of fusion-competent gp120/gp41. When evaluated in humans, recombinant gp160 molecules offered no advantages over gp120 monomers (Gorse, 1998).

Uncleaved gp140 (gp140UNC): Stable “oligomers” have been made by eliminating the natural proteolytic site needed for conversion of the gp160 precursor protein into gp120 and gp41 (Berman, 1989; and Earl, 1990). To express these constructs as soluble proteins, a stop codon is inserted within the env gene to truncate the protein immediately prior to the transmembrane-spanning segment of gp41. The protein lacks the transmembrane domain and the long, intra-cytoplasmic tail of gp41, but retains the regions important for virus entry and the induction of neutralizing antibodies. The secreted protein contains full-length gp120 covalently linked through a peptide bond to the ectodomain of gp41. The protein migrates in SDS-PAGE as a single species with an apparent molecular mass of approximately 140 kilodaltons (kDa) under both reducing and nonreducing conditions. The protein forms higher molecular weight noncovalent oligomers, likely through interactions mediated by the gp41 moieties.

Several lines of evidence suggest that the uncleaved gp140 molecule does not adopt the same conformation as native gp120-gp41. These include observations that uncleaved gp120-gp41 complexes do not avidly bind fusion co-receptors. Furthermore, a gp140 protein was unable to efficiently select for neutralizing MAbs when used to pan a phage-display library, whereas virions were efficient (Parren, 1996). We refer to the uncleaved gp120-gp41 ectodomain material as gp140UNC.

Cleavable but uncleaved gp140 (gp140NON): During biosynthesis, gp160 is cleaved into gp120 and gp41 by a cellular endoprotease of the furin family. Mammalian cells have a finite capacity to cleave gp120 from gp41. Thus, when over-expressed, the envelope glycoproteins can saturate the endogenous furin enzymes and be secreted in precursor form. Since these molecules are potentially cleavable, we refer to them as gp140NON. Like gp140UNC, gp140NON migrates in SDS-PAGE with an apparent molecular mass of approximately 140 kDa under both reducing and nonreducing conditions. gp140NON appears to possess the same non-native topology as gp140UNC.

Cleaved gp140 (gp140CUT): gp140CUT refers to full-length gp120 and ectodomain gp41 fully processed and capable of forming oligomers as found on virions. The noncovalent interactions between gp120 and gp41 are sufficiently long-lived for the virus to bind and initiate fusion with new target cells, a process which is likely completed within minutes during natural infection. The association has, however, to date proven too labile for the production of significant quantities of cleaved gp140s in near homogenous form.

Stabilization of Viral Envelope Glycoproteins

The metastable pre-fusion conformation of viral envelope proteins such as gp120/gp41 has evolved to be sufficiently stable so as to permit the continued spread of infection yet sufficiently labile to readily allow the conformational changes required for virus-cell fusion. For the HIV isolates examined thus far, the gp120-gp41 interaction has proven too unstable for preparative-scale production of gp140CUT as a secreted protein. Given the enormous genetic diversity of HIV, however, it is conceivable that viruses with superior env stability could be identified using screening methods such as those described herein. Alternatively, viruses with heightened stability could in principle be selected following successive exposure of virus to conditions known to destabilize the gp120-gp41 interaction. Such conditions might include elevated temperatures in the range of 37-60° C. and/or low concentrations of detergents or chaotropic agents. The envelope proteins from such viruses could be subcloned into the pPPI4 expression vector and analyzed for stability using our methods as well.

One could also adopt a semi-empirical, engineered approach to stabilizing viral envelope proteins. For example stable heterodimers have been successfully created by introducing complementary “knob” and “hole” mutations in the binding partners (Atwell, 1997). Alternatively or in addition, one could introduce other favorable interactions, such as salt bridges, hydrogen bonds, or hydrophobic interactions. This approach is facilitated by increased understanding of the structures of the surface (SU) and transmembrane (TM) proteins.

SU-TM stabilization can also be achieved by means of one or more introduced disulfide bonds. Among mammalian retroviruses, only the lentiviruses such as HIV have non-covalent associations between the SU and TM glycoproteins. In contrast, the type C and type D retroviruses all have an inter-subunit disulfide bond. The ectodomains of retroviral TM glycoproteins have a broadly common structure, one universal feature being the presence of a small, Cys-Cys bonded loop approximately central in the ectodomain. In the type C and D retroviral TM glycoproteins, an unpaired cysteine residue is found immediately C-terminal to this loop and is almost certainly used in forming the SU-TM disulfide bond (Gallaher, 1995; and Schultz, 1992).

Although gp41 and other lentiviral TM glycoproteins lack the third cysteine, the structural homologies suggest that one could be inserted in the vicinity of the short central loop structure. Thus there is strong mutagenic evidence that the first and last conserved regions of gp120 (C1 and C5 domains) are probable contact sites for gp41.

II. Particle Vaccines

Studies have revealed the advantage that is conferred by converting a soluble protein into a particulate form in the preparation of a vaccine. Precipitated aluminum salts or “alum” remain the only adjuvant utilized in vaccines licensed for human use by the United States Food and Drug Administration. Several other particulate adjuvants have been tested in animals. The major examples include beads prepared from poly(lactic-co-glycolic acid) [PLG] (Cleland, 1994; Hanes, 1997; and Powell, 1994), polystyrene (Kovacsovics-Bankowski, 1995; Raychaudhuri, 1998; Rock, 1996; and Vidard, 1996), liposomes (Alving, 1995), calcium phosphate (He, 2000), and cross-linked or crystallized proteins (Langhein, 1987; and St. Clair, 1999).

In one series of studies, ovalbumin was linked to polystyrene beads (Vidard, 1996). These studies revealed that antigen-specific B cells can bind particulate antigens directly via their surface Ig receptor, enabling them to phagocytose the antigen, process it, and present the resulting peptides to T cells. The optimum size for particulate antigen presentation in this context was found to be 4 μm. Other studies with biodegradable PLG microspheres between 1 and 10 μm in diameter show that these particles are capable of delivering antigens into the major histocompatibility complex (MHC) class I pathway of macrophages and dendritic cells and are able to stimulate strong cytotoxic T lymphocyte (CTL) responses in vivo (Raychaudhuri, 1998). PLG microspheres containing internalized ovalbumin and other antigens also induced humoral immune responses that were greater than those achieved with soluble antigen alone (Men, 1996; and Partidos, 1996).

The potent, long-lasting immune responses induced after a single immunization with antigen-loaded or antigen-coated microspheres may result from multiple mechanisms: efficient phagocytosis of the small (<10 μm) particles, which results in their transport to lymph nodes, antigen processing and presentation to T-helper cells; the gradual release of antigens from the surface or interior of the particles, leading to the stimulation of immune-competent cells; and the sustained presentation of surface antigen (Coombes, 1999; Coombes, 1996; and O'Hagan, 1993). Antigen-presenting cells (APCs) localize to antigen-specific B cells under these conditions, and release cytokines that increase specific antibody production and augment the expansion of these antigen specific B-cell clones. Particulate antigens are also useful for generating mucosal humoral immunity by virtue of their ability to induce secretory IgA responses after mucosal vaccination (O'Hagan, 1993; and Vidard, 1996).

Overall, the use of particulate antigens allows for the simultaneous activation of both the humoral and cell-mediated arms of the immune response by encouraging the production of antigen-specific antibodies that opsonize particulate antigens and by causing the antigens to be phagocytosed and shunted into the MHC Class I antigen presentation pathway (Kovacsovics, 1995; Raychaudhuri, 1998; Rock, 1996; and Vidard, 1996).

Typically, the antigens are attached to the particles by physical adsorption. Antigens have also been incorporated into particles by entrapment, as is commonly performed for PLG-based vaccines (Hanes, 1997). More rarely, the antigens are covalently linked to functional groups on the particles (Langhein, 1987).

### SUMMARY OF THE INVENTION

This invention provides a first composition comprising a pharmaceutically acceptable particle and a stable HIV-1 pre-fusion envelope glycoprotein trimeric complex operably affixed thereto, each monomeric unit of the complex comprising HIV-1 gp120 and HIV-1 gp41, wherein (i) the gp120 and gp41 are bound to each other by at least one disulfide bond between a cysteine residue introduced into the gp120 and a cysteine residue introduced into the gp41, and (ii) the gp120 has deleted from it at least one V-loop present in wild-type HIV-1 gp120.

This invention further provides a method for eliciting an immune response in a subject against HIV-1 or an HIV-1-infected cell comprising administering to the subject a prophylactically or therapeutically effective amount of the first composition.

This invention further provides a vaccine which comprises a therapeutically effective amount of the first composition and a pharmaceutically acceptable carrier.

This invention further provides a vaccine which comprises a prophylactically effective amount of the first composition and a pharmaceutically acceptable carrier.

This invention further provides a method for preventing a subject from becoming infected with HIV-1 comprising administering to the subject a prophylactically effective amount of the first composition, thereby preventing the subject from becoming infected with HIV-1.

This invention further provides a method for reducing the likelihood of a subject's becoming infected with HIV-1 comprising administering to the subject a prophylactically effective amount of the first composition, thereby reducing the likelihood of the subject's becoming infected with HIV-1.

This invention further provides a method for preventing or delaying the onset of, or slowing the rate of progression of, an HIV-1-related disease in an HIV-1-infected subject which comprises administering to the subject a therapeutically effective amount of the first composition, thereby preventing or delaying the onset of, or slowing the rate of progression of, the HIV-1-related disease in the subject.

This invention further provides a method for producing the first composition, comprising contacting a pharmaceutically acceptable particle with a stable HIV-1 pre-fusion envelope glycoprotein trimeric complex under conditions permitting the complex to become operably affixed to the particle, wherein each monomeric unit of the complex comprises HIV-1 gp120 and HIV-1 gp41, (i) the gp120 and gp41 being bound to each other by at least one disulfide bond between a cysteine residue introduced into the gp120 and a cysteine residue introduced into the gp41, and (ii) the gp120 having deleted from it at least one V-loop present in wild-type HIV-1 gp120.

This invention further provides a second method for producing the first composition, comprising contacting (a) a pharmaceutically acceptable particle having operably affixed thereto an agent which binds to a stable HIV-1 pre-fusion envelope glycoprotein trimeric complex and (b) a stable HIV-1 pre-fusion envelope glycoprotein trimeric complex under conditions permitting the complex to bind to the agent, thereby permitting the complex to become operably affixed to the particle, wherein each monomeric unit of the complex comprises HIV-1 gp120 and HIV-1 gp41, (i) the gp120 and gp41 being bound to each other by at least one disulfide bond between a cysteine residue introduced into the gp120 and a cysteine residue introduced into the gp41, and (ii) the gp120 having deleted from it at least one V-loop present in wild-type HIV-1 gp120.

This invention further provides a method for isolating a stable HIV-1 pre-fusion envelope glycoprotein trimeric complex comprising contacting, under suitable conditions, a stable HIV-1 pre-fusion envelope glycoprotein trimeric complex-containing sample with a pharmaceutically acceptable particle having operably affixed thereto an agent which specifically binds to the trimeric complex, wherein each monomeric unit of the complex comprises HIV-1 gp120 and HIV-1 gp41, (i) the gp120 and gp41 being bound to each other by at least one disulfide bond between a cysteine residue introduced into the gp120 and a cysteine residue introduced into the gp41, and (ii) the gp120 having deleted from it at least one V-loop present in wild-type HIV-1 gp120; and separating the particle from the sample, thereby isolating the trimeric complex.

This invention further provides a second composition comprising (a) a pharmaceutically acceptable particle, (b) an antigen, and (c) an agent which is operably affixed to the particle and is specifically bound to the antigen, whereby the antigen is operably bound to the particle.

This invention further provides a method for eliciting an immune response against an antigen in a subject comprising administering to the subject a prophylactically or therapeutically effective amount of the second composition, wherein the composition comprises the antigen against which the immune response is elicited operatively bound to the particle of the composition.

This invention also provides a vaccine which comprises a therapeutically effective amount of the second composition and a pharmaceutically acceptable carrier. This invention further provides a vaccine which comprises a prophylactically effective amount of the second composition and a pharmaceutically acceptable carrier.

This invention further provides a method for preventing a subject from becoming infected with a virus comprising administering to the subject a prophylactically effective amount of the second composition, wherein the antigen of the composition is present on the surface of the virus, thereby preventing the subject from becoming infected with the virus.

This invention further provides a method for reducing the likelihood of subject's becoming infected with a virus comprising administering to the subject a prophylactically effective amount of the second composition, wherein the antigen of the composition is present on the surface of the virus, thereby reducing the likelihood of the subject's becoming infected with the virus.

This invention further provides a method for preventing or delaying the onset of, or slowing the rate of progression of, a virus-related disease in a virus-infected subject comprising administering to the subject a therapeutically effective amount of the second composition, wherein the antigen of the composition is present on the surface of the virus, thereby preventing or delaying the onset of, or slowing the rate of progression of, the virus-related disease in the subject.

This invention further provides a method for producing the second composition, comprising contacting (a) a pharmaceutically acceptable particle having operably affixed thereto an agent which specifically binds to an antigen and (b) the antigen, under conditions permitting the antigen to bind the agent, thereby permitting the antigen to become operably affixed to the particle.

This invention further provides a method for eliciting an immune response against a tumor-specific antigen in a subject comprising administering to the subject a prophylactically or therapeutically effective amount of the second, tumor-related composition.

This invention further provides a method for preventing the growth of, or slowing the rate of growth of, a tumor in a subject comprising administering to the subject a therapeutically effective amount of the second, tumor-related composition, wherein the tumor-associated antigen of the composition is present on the surface of cells of the tumor, thereby preventing the growth of, or slowing the rate of growth of, the tumor in the subject.

Finally, this invention further provides a method for reducing the size of a tumor in a subject comprising administering to the subject a therapeutically effective amount of the second, tumor-related composition, wherein the tumor-associated antigen of the composition is present on the surface of cells of the tumor, thereby reducing the size of the tumor in the subject.

### BRIEF DESCRIPTION OF THE FIGURES

FIG. 1

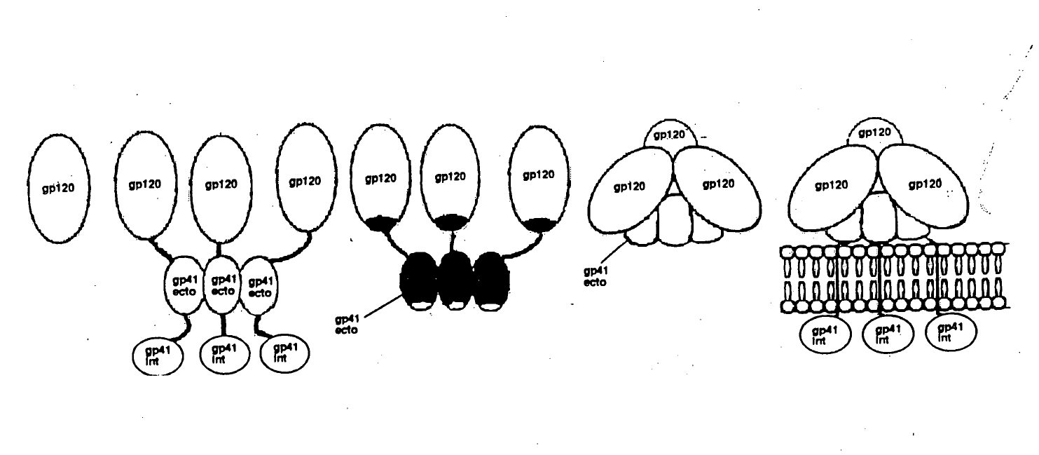

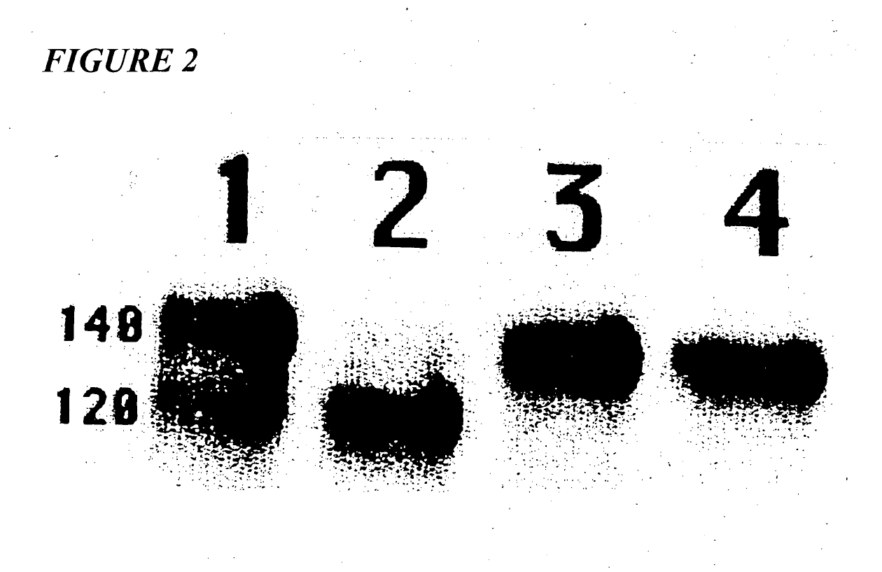

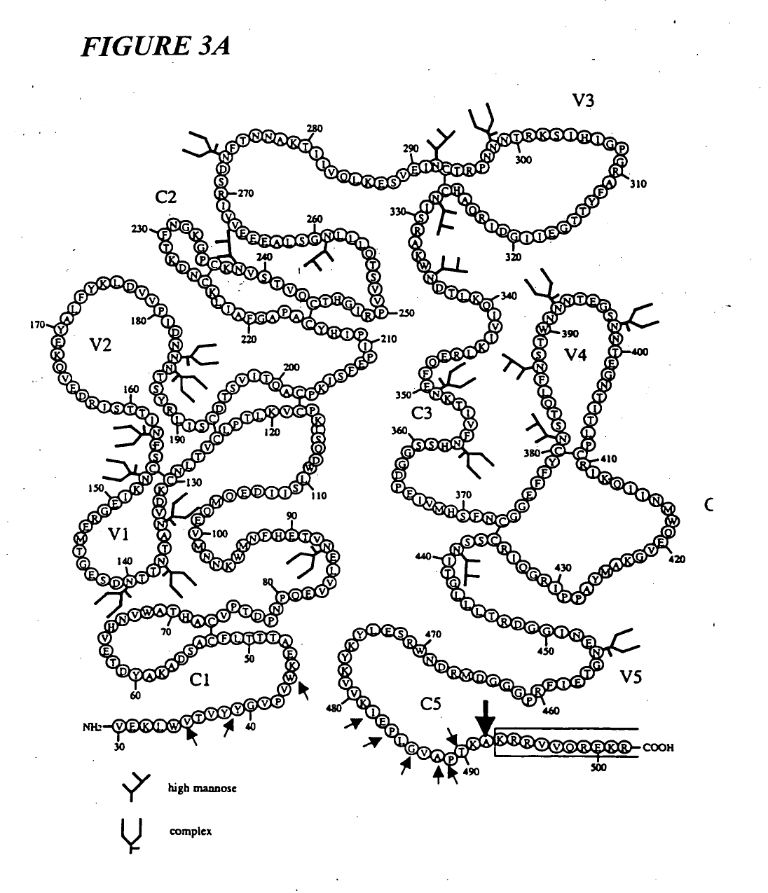

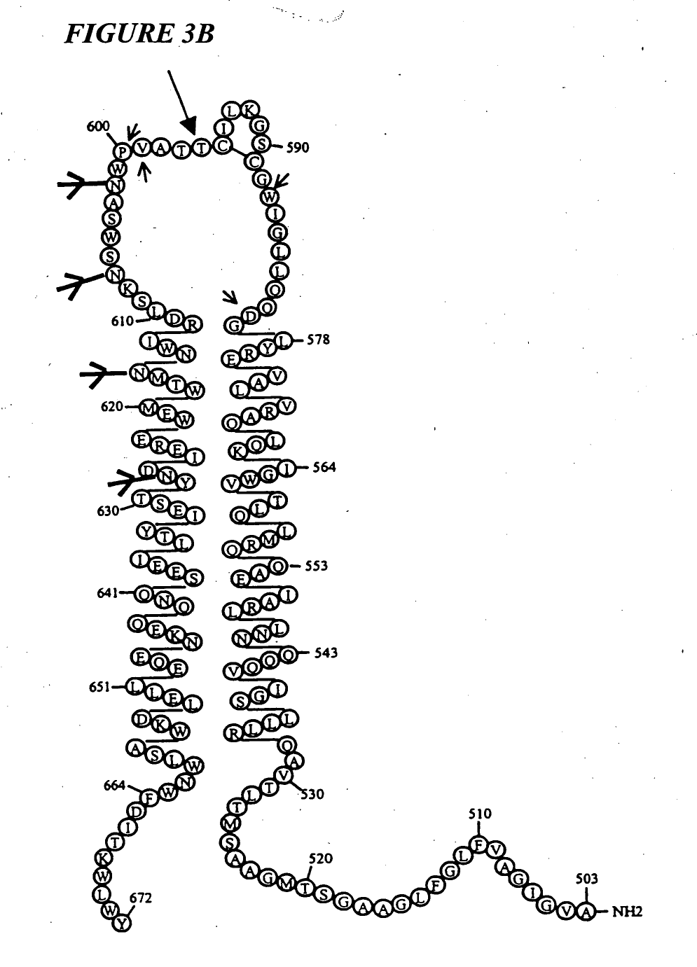

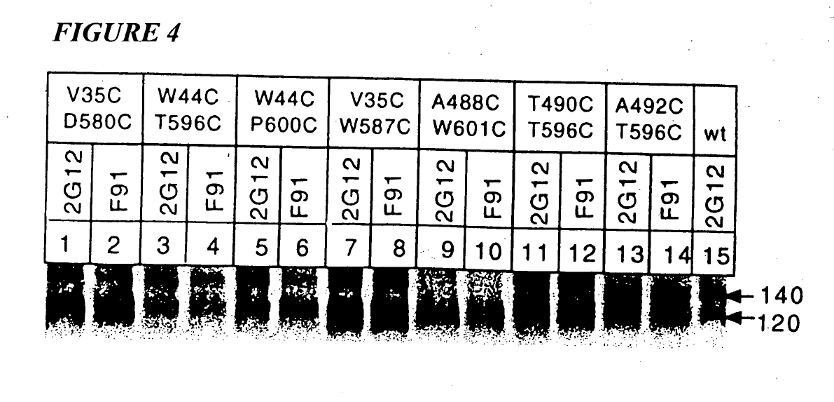

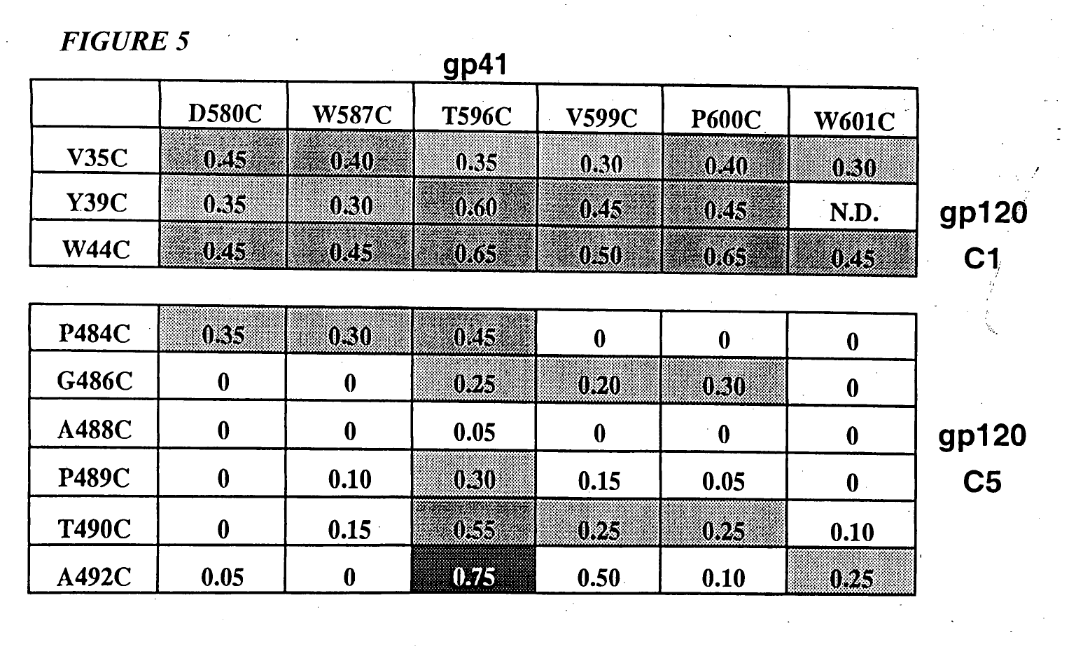

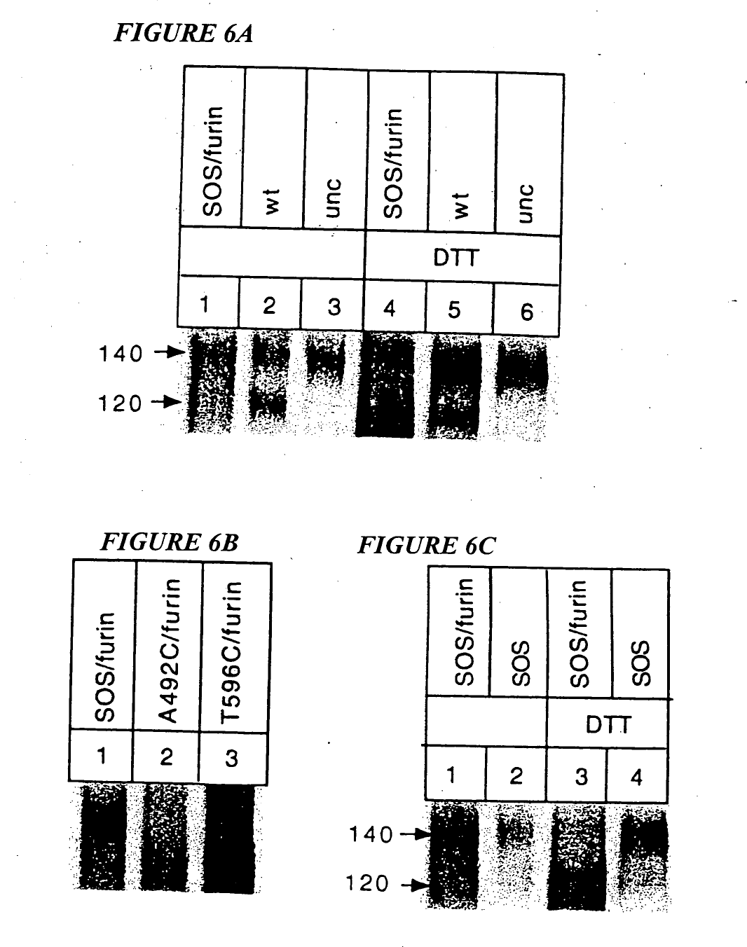

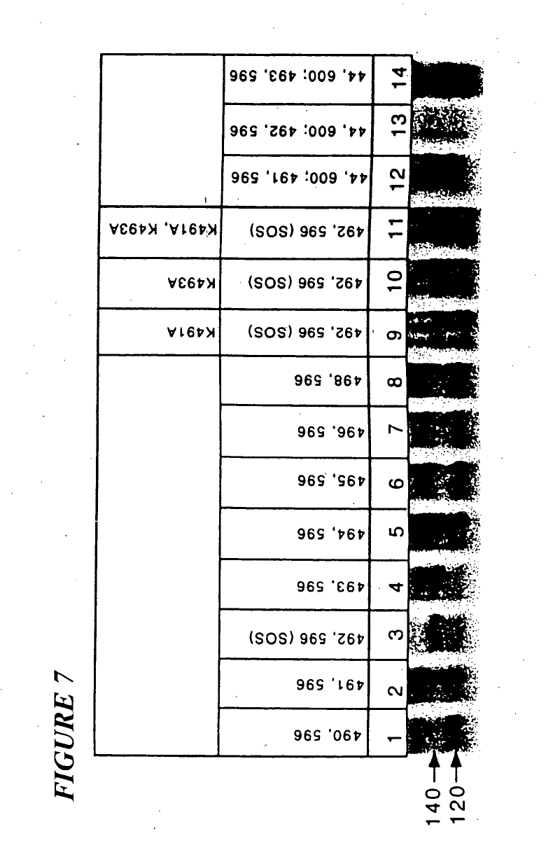

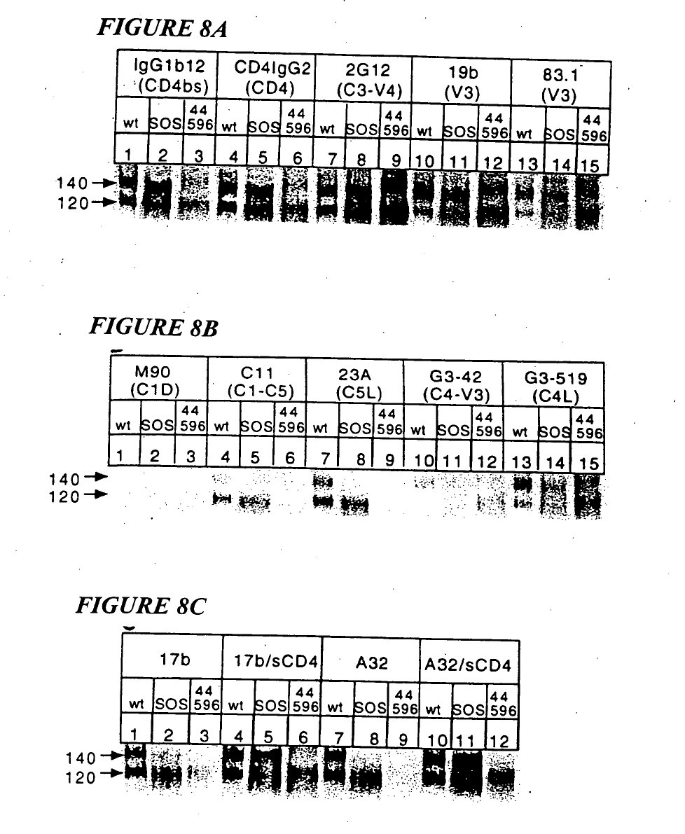

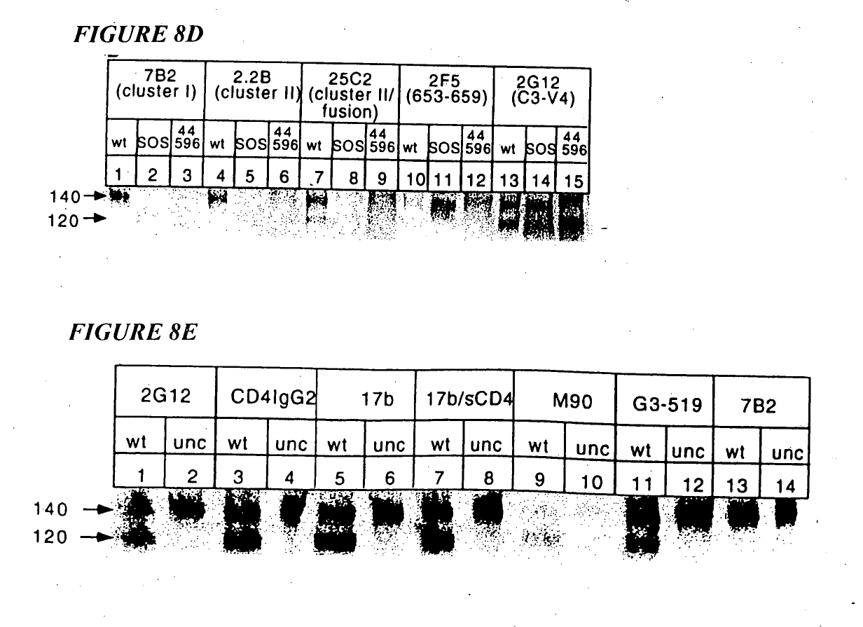

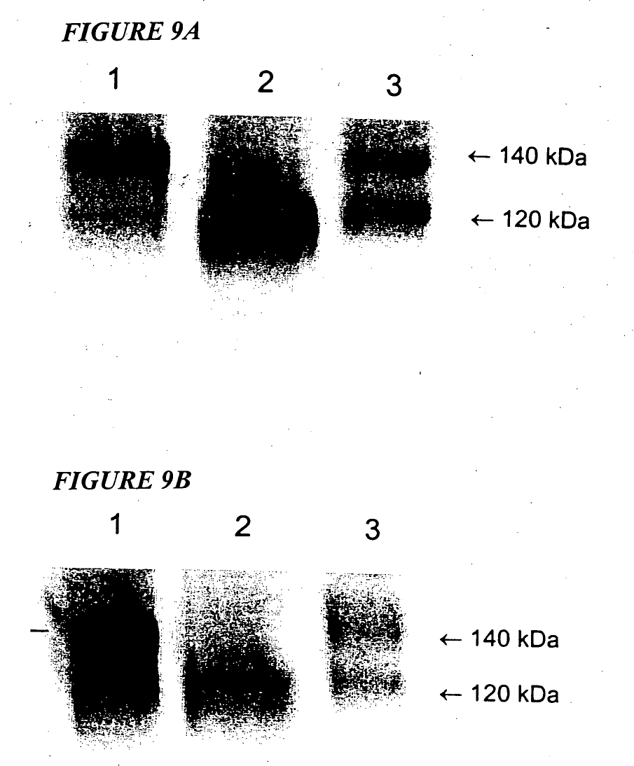

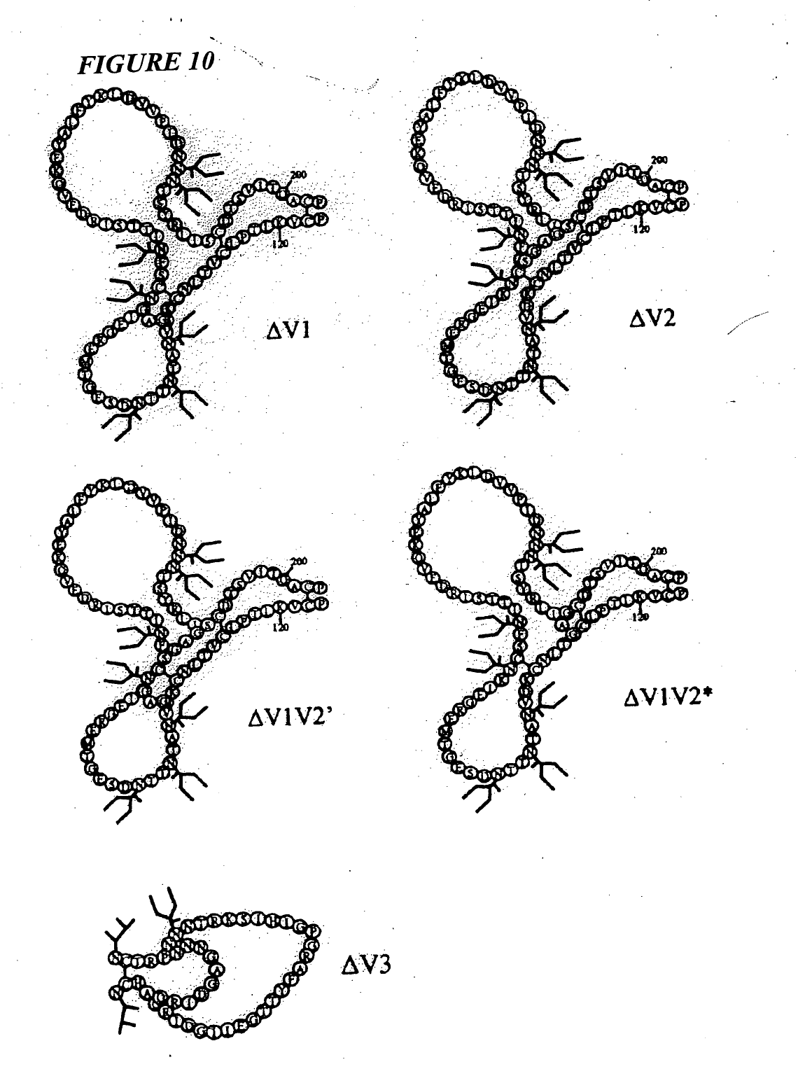

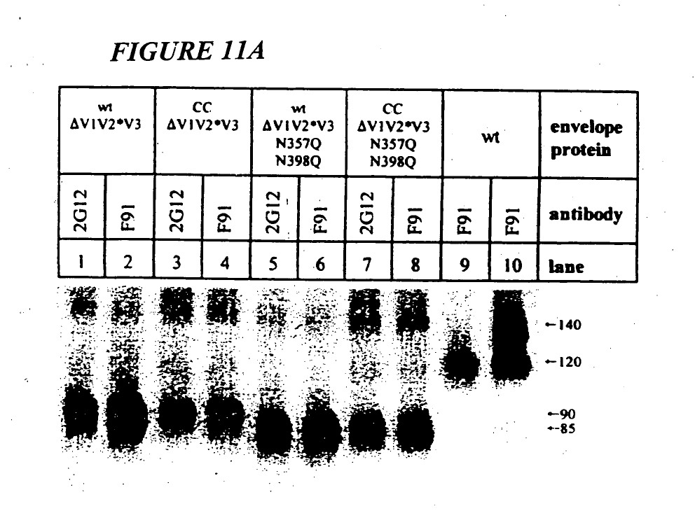

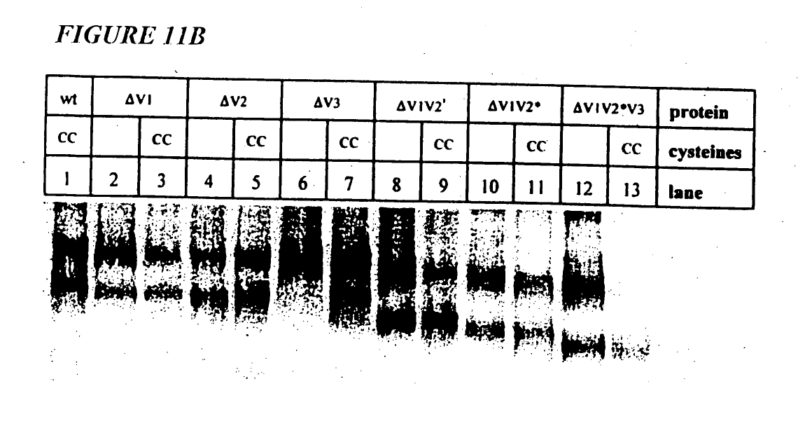

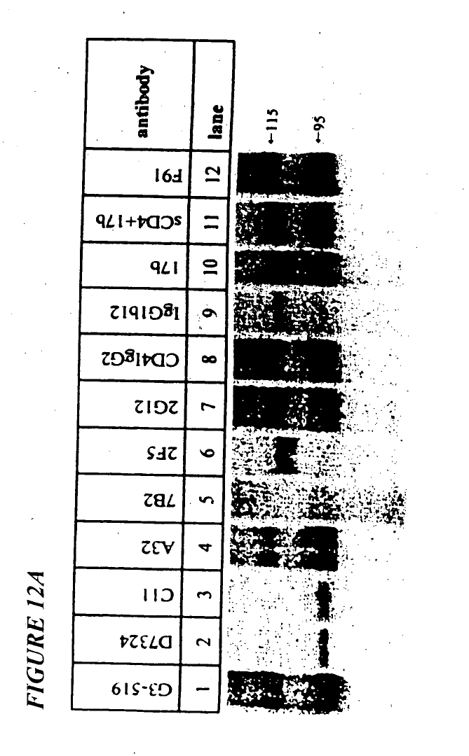

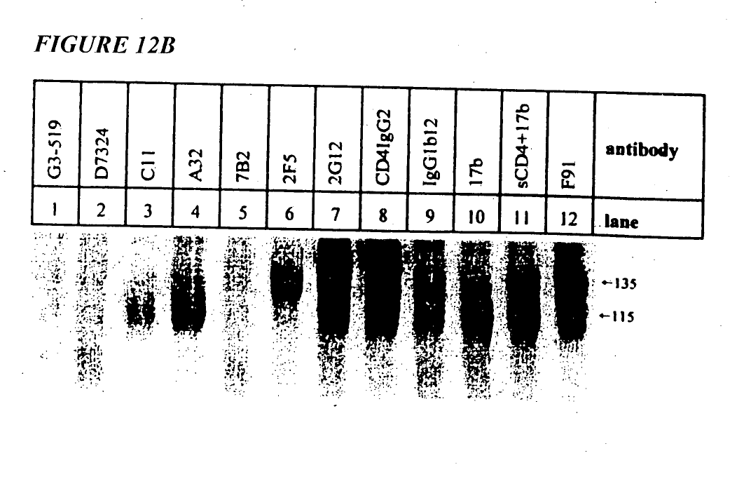

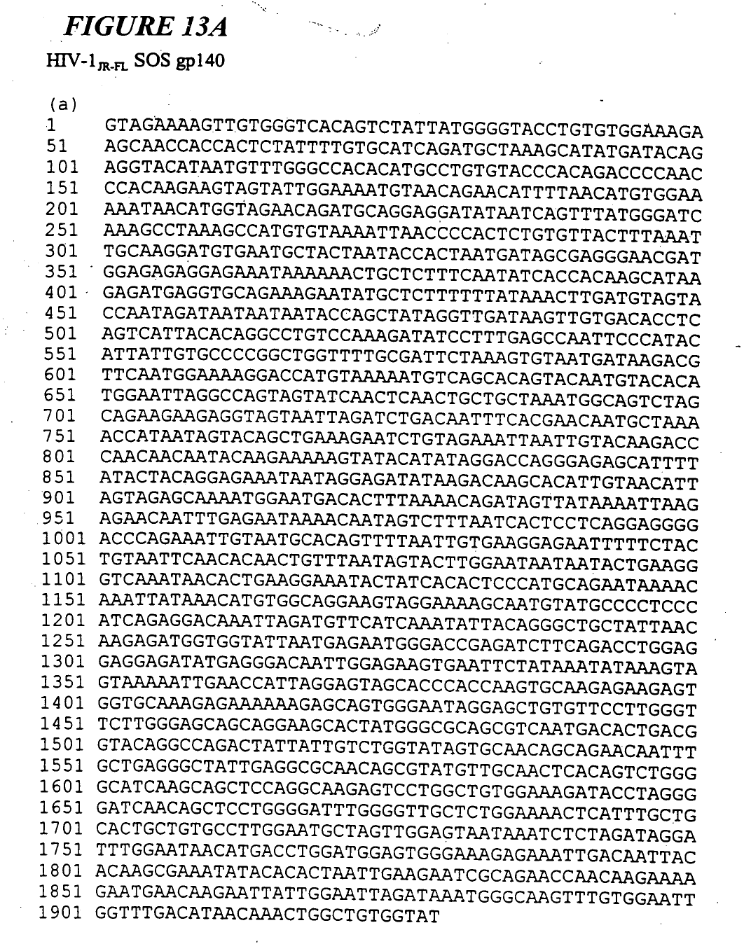

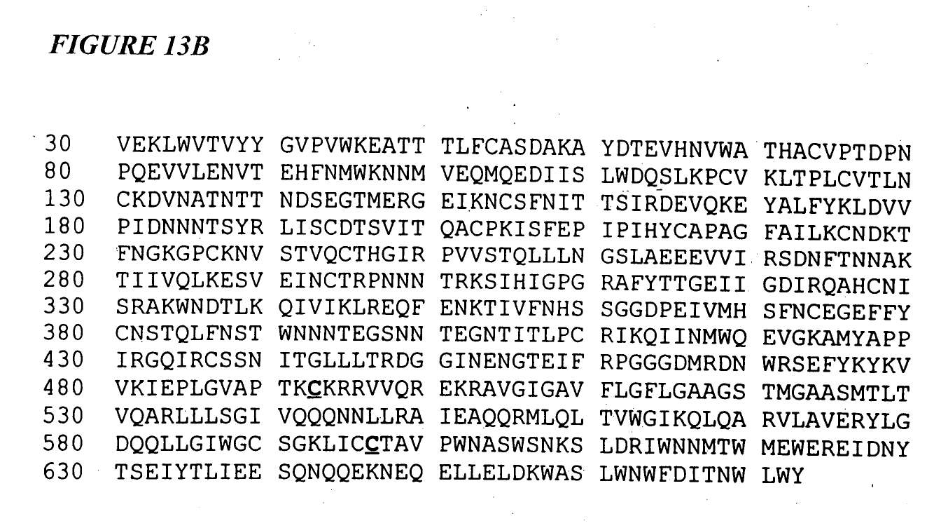

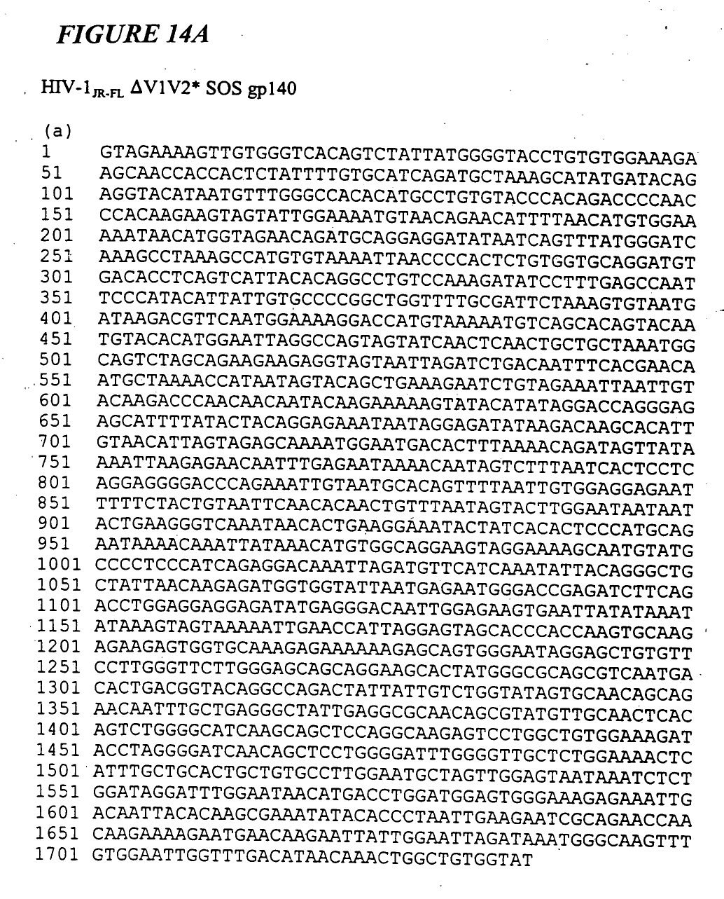

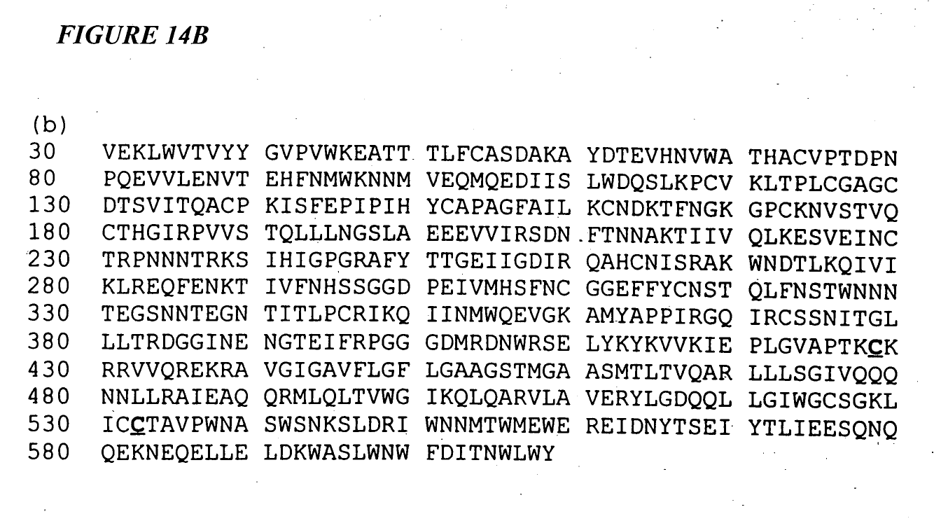

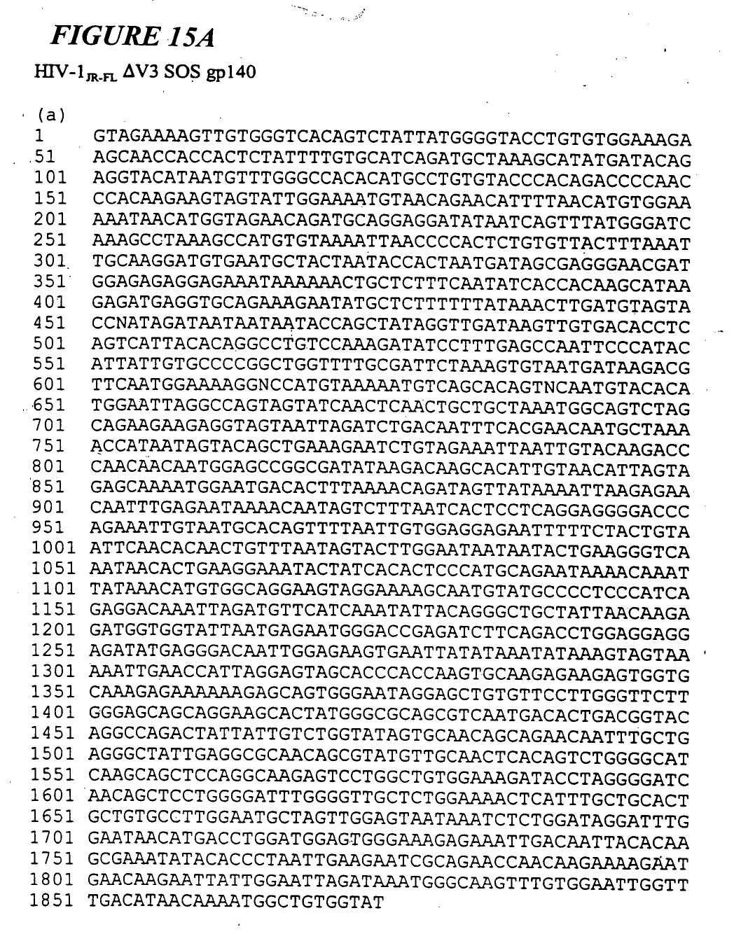

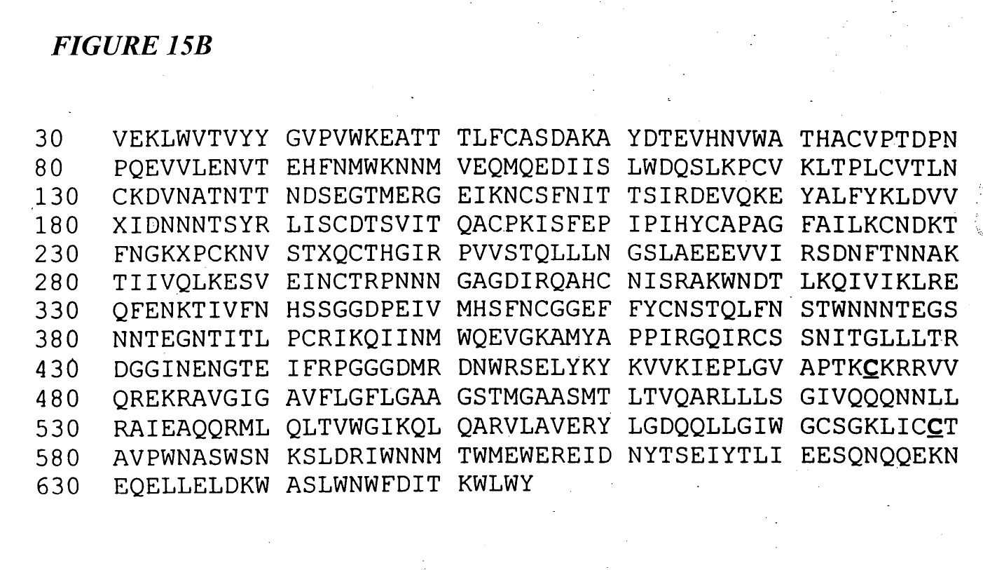

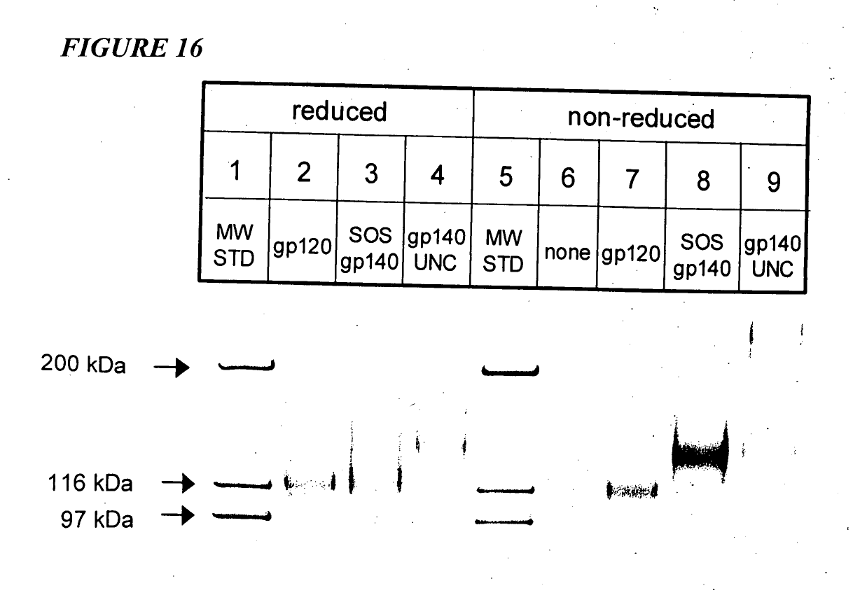

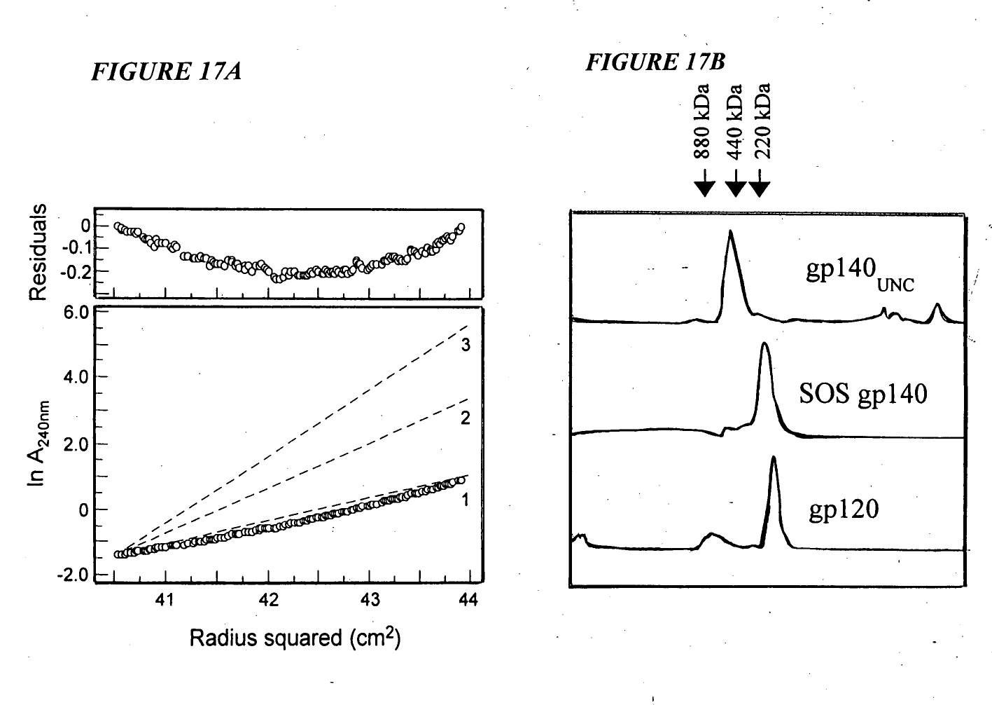

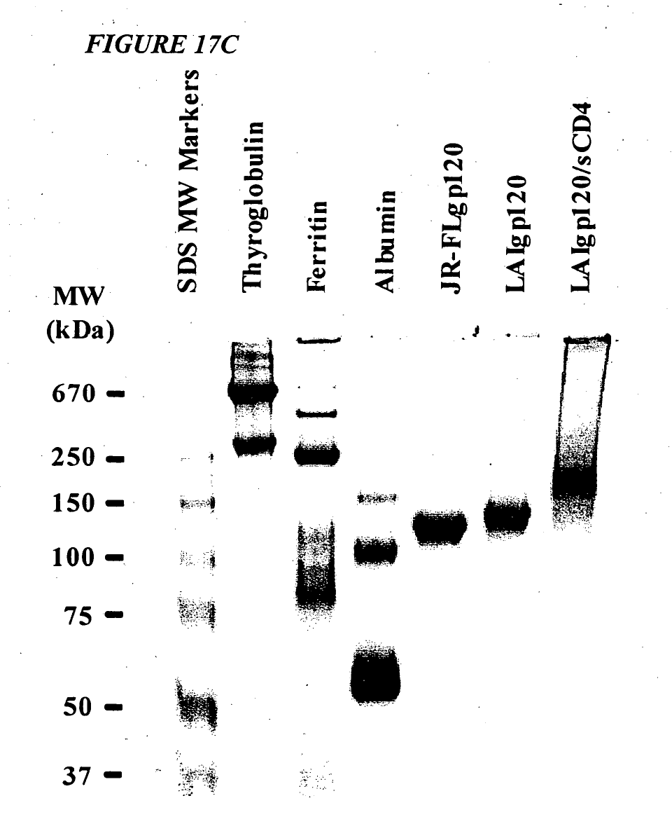

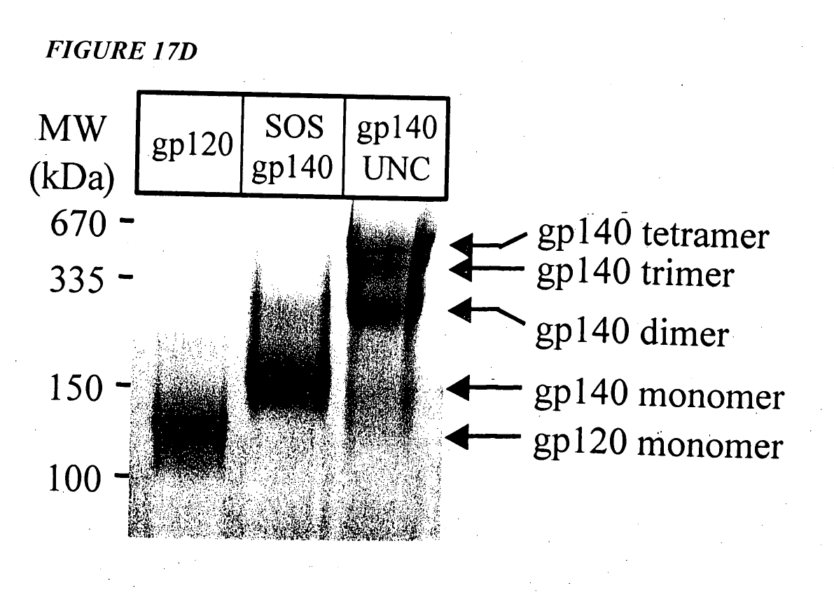

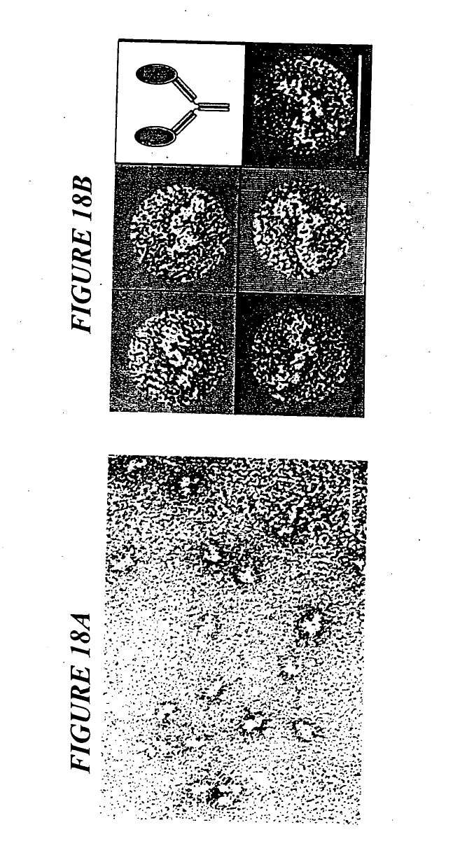

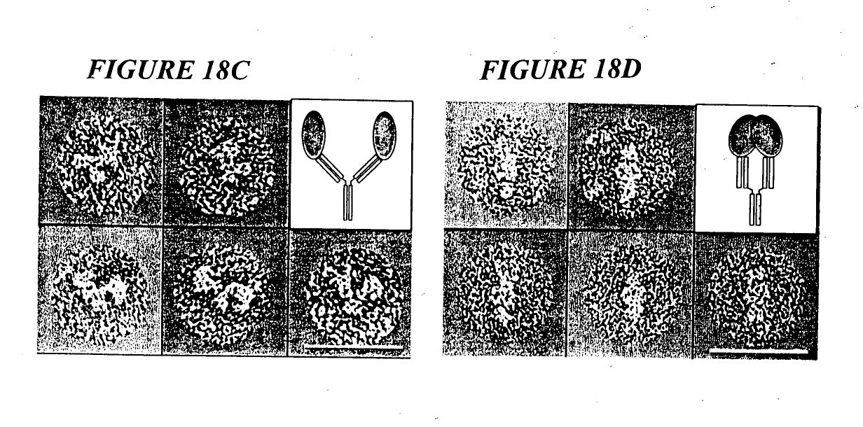

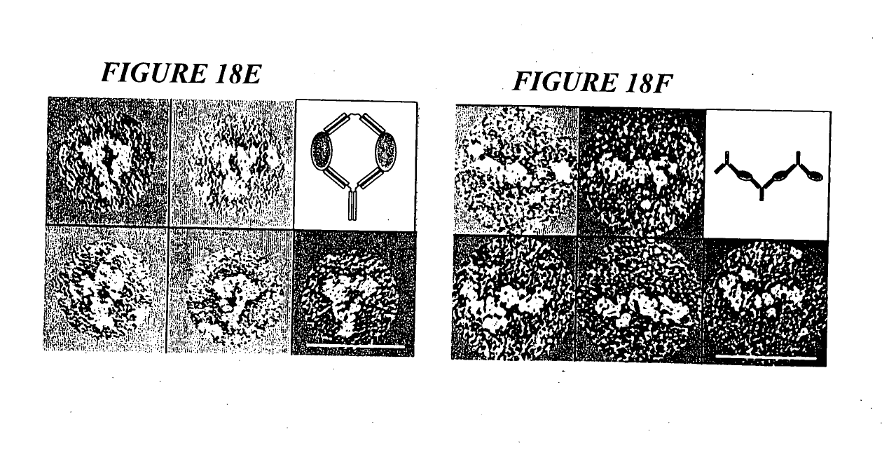

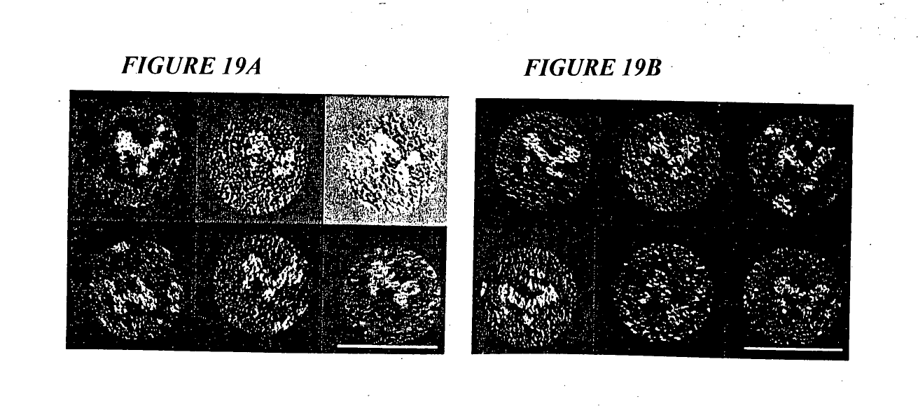

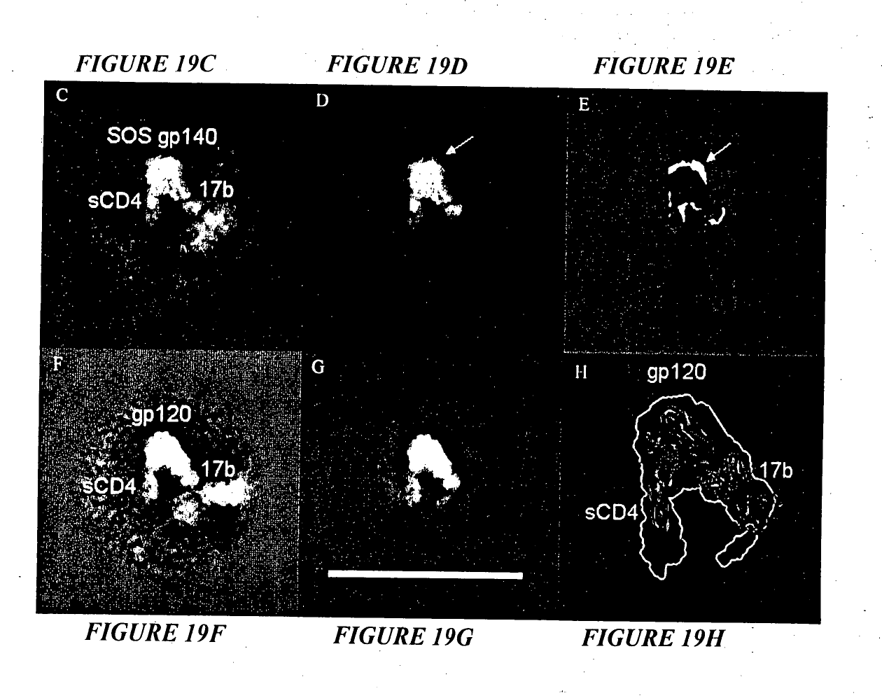

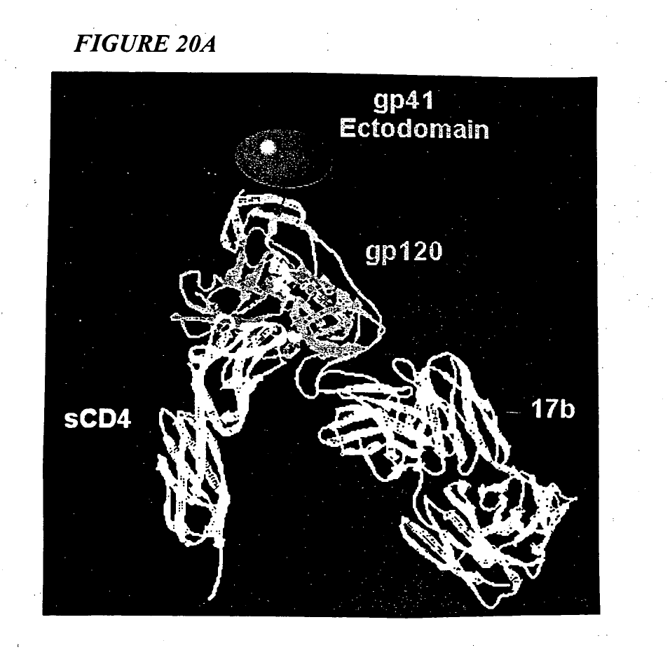

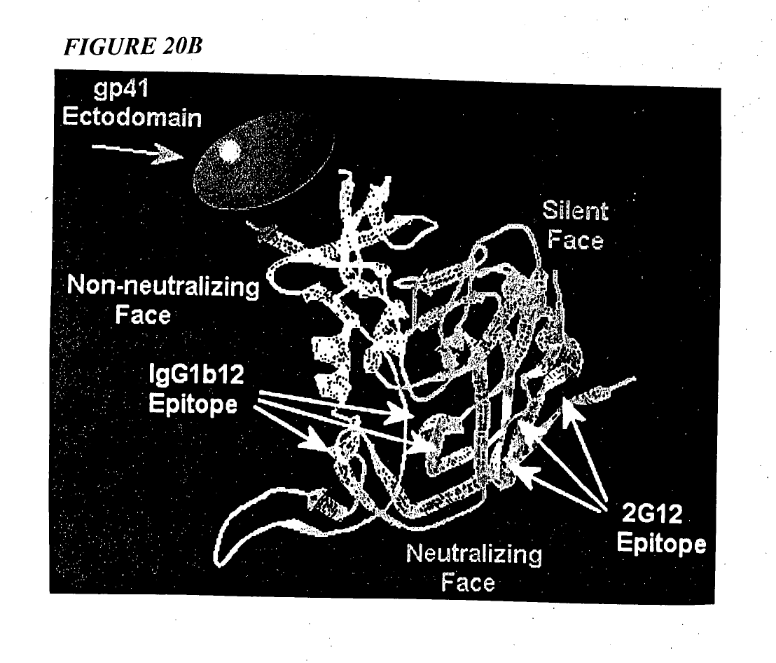

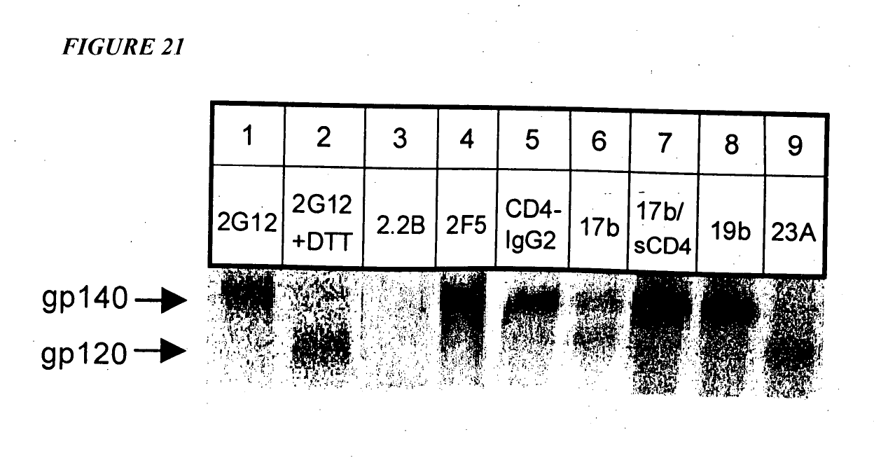

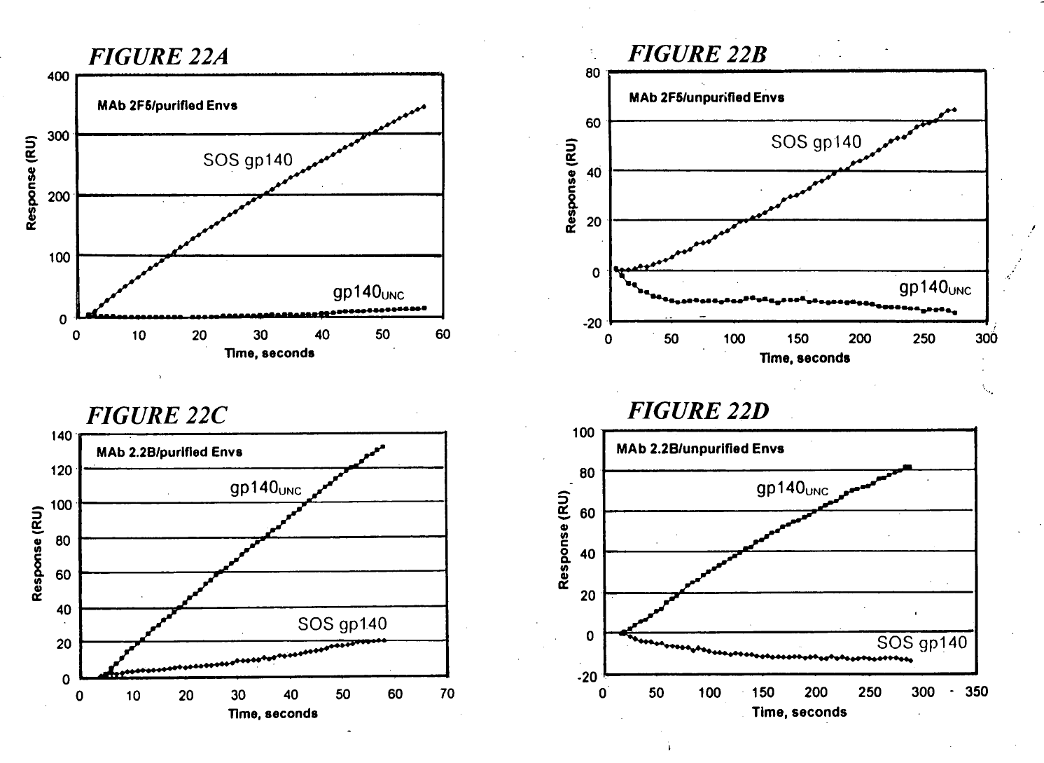

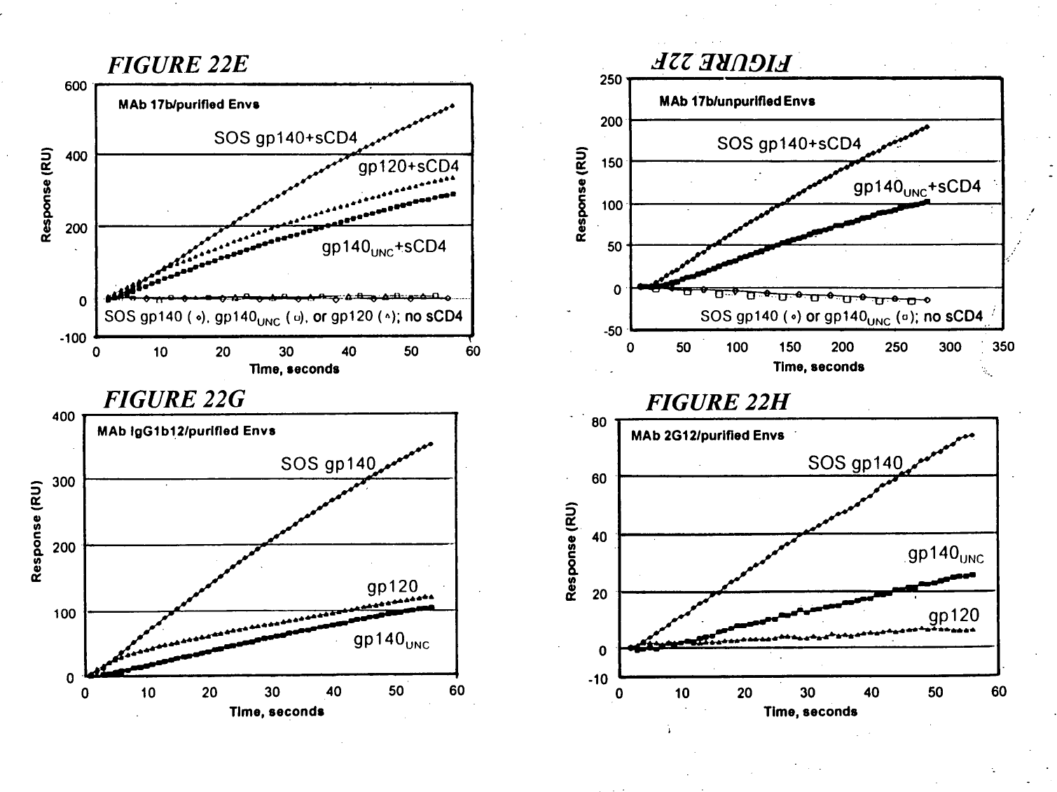

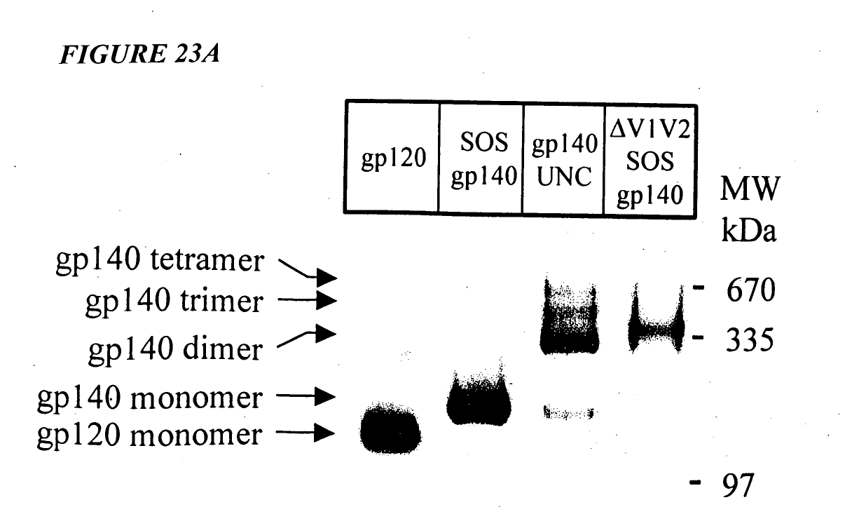

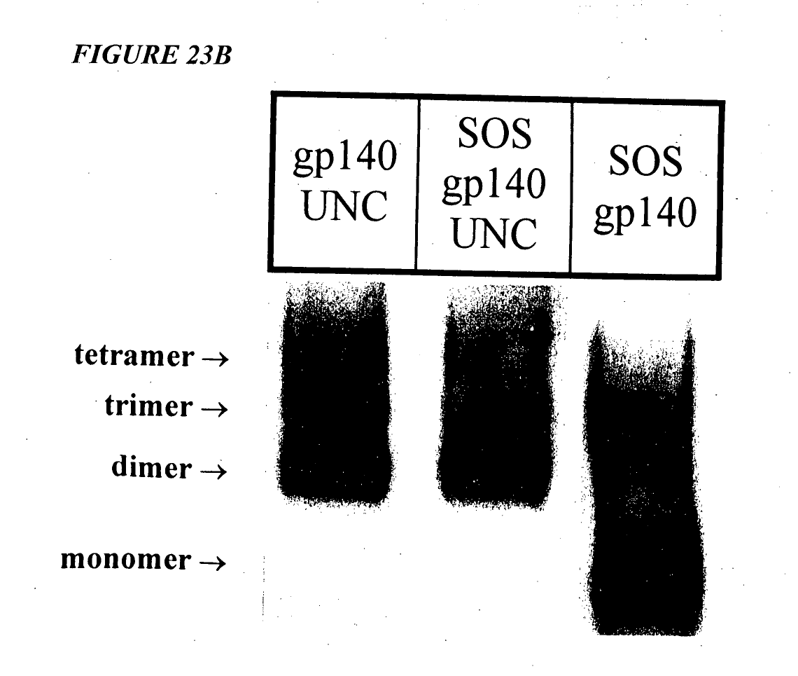

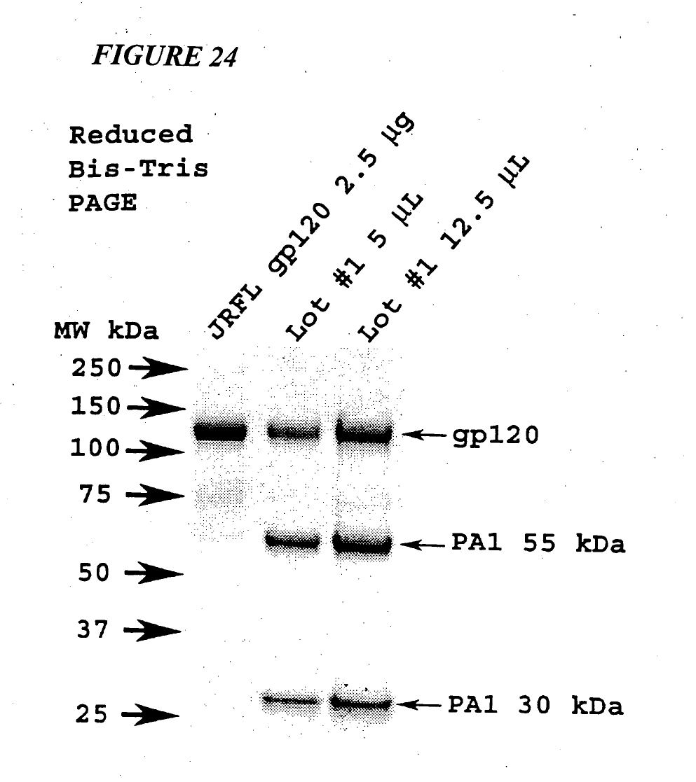

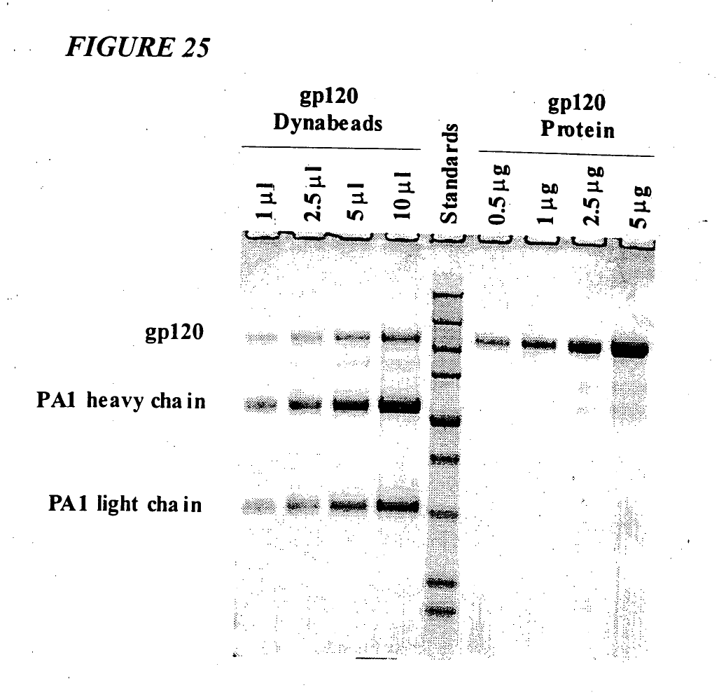

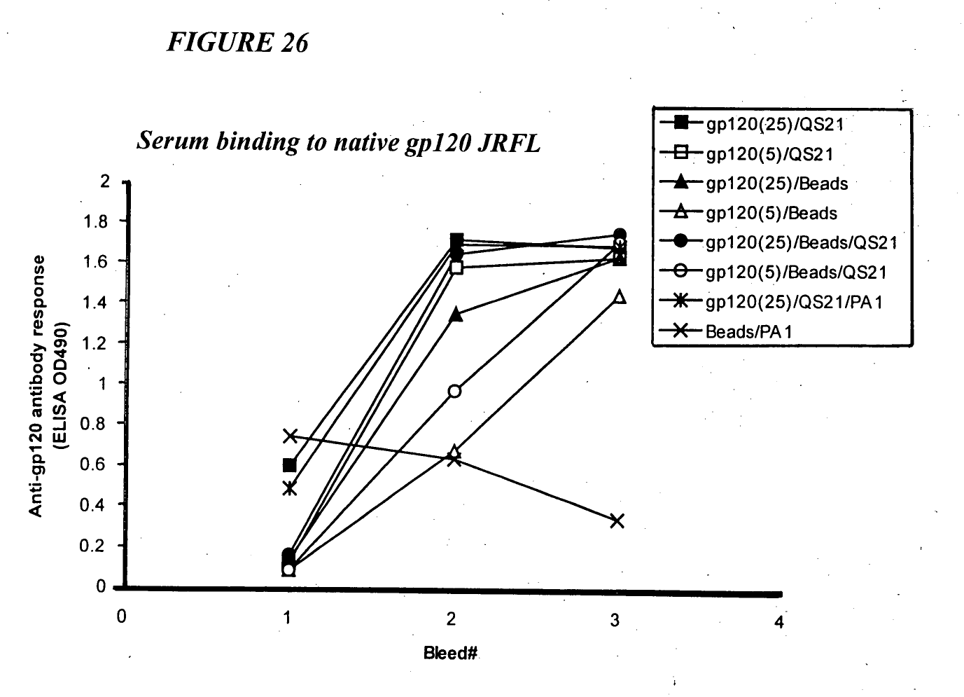

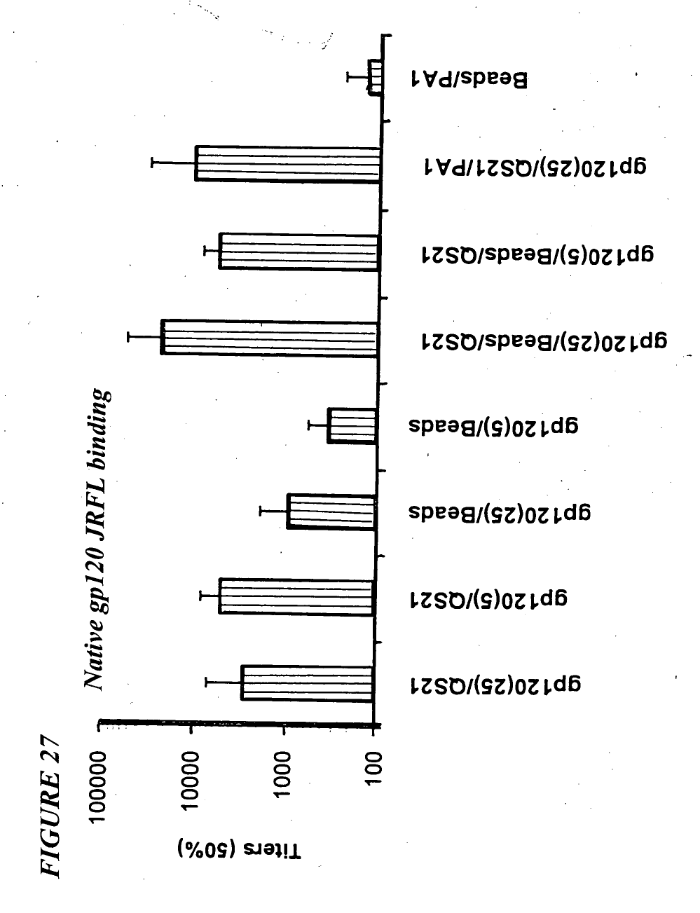

Different forms of the HIV-1 envelope glycoproteins. The cartoons depict: i) Monomeric gp120; ii) Full-length recombinant gp160; iii) Proteolytically unprocessed gp140 trimer with the peptide bond maintained between gp120 and gp41 (gp140UNC or gp140NON); iv) The SOS gp140 protein, a proteolytically processed gp140 stabilized by an intermolecular disulfide bond; and v) Native, virion-associated gp120-gp41 trimer. The shading of the gp140UNC protein (iii) indicates the major antibody-accessible regions that are poorly, or not, exposed on the SOS gp140 protein or on the native gp120-gp41 trimer.

FIG. 2

Co-transfection of furin increases the efficiency of cleavage of the peptide bond between gp120 and gp41. 293T cells were transfected with DNA expressing HIV-1_{JR-FL} gp140 wild-type (WT) or gp140UNC (gp120-gp41 cleavage site mutant) proteins, in the presence or absence of a co-transfected furin-expressing plasmid. The ³⁵S-labelled envelope glycoproteins secreted from the cells were immunoprecipitated with the anti-gp120 MAb 2G12, then analyzed by SDS-PAGE. Lane 1, gp140WT (gp140/gp120 doublet); Lane 2, gp140WT plus furin (gp120 only); Lane 3, gp140UNC (gp140 only); Lane 4, gp140UNC plus furin (gp140 only). The approximate molecular weights, in kDa, of the major species are indicated on the left.

FIG. 3

Positions of cysteine substitutions in JR-FL gp140. The various residues of the JR-FL gp140WT protein that have been mutated to cysteines in one or more mutants are indicated by closed arrows on the schematics of the gp120 and gp41ECTO subunits. The positions of the alanine-492 and threonine-596 residues that are both mutated to cysteine in the SOS gp140 protein are indicated by the larger, closed arrows. (a) JR-FL gp120. (b) JR-FL gp41. The open boxes at the C-terminus of gp120 and the N-terminus of gp41 indicate the regions that are mutated in the gp140UNC protein to eliminate the cleavage site between gp120 and gp41.

FIG. 4

Immunoprecipitation analysis of selected double cysteine mutants of JR-FL gp140. The ³⁵S-labelled envelope glycoproteins secreted from transfected 293T cells were immunoprecipitated with anti-gp120 and anti-gp41 MAbs, then analyzed by SDS-PAGE. The MAbs used were either 2G12 (anti-gp120 C3-V4 region) or F91 (anti-gp120 CD4 binding site region).

The positions of the two cysteine substitutions in each protein (one in gp120, the other in gp41ECTO) are noted above the lanes. The gp140WT protein is shown in lane 15. All proteins were expressed in the presence of co-transfected furin, except for the gp140WT protein.

FIG. 5

The efficiency of intermolecular disulfide bond formation is dependent upon the positions of the cysteine substitutions. The ³⁵S-labelled envelope glycoproteins secreted from 293T cells co-transfected with furin and the various gp140 mutants were immunoprecipitated with the anti-gp120 MAb 2G12, then analyzed by SDS-PAGE. For each mutant, the intensities of the 140 kDa and 120 kDa bands were determined by densitometry and the gp140/gp140+gp120 ratio was calculated and recorded. The extent of shading is proportional to the magnitude of the gp140/gp140+gp120 ratio. The positions of the amino acid substitutions in gp41 and the C1 and C5 domains of gp120 are recorded along the top and down the sides, respectively. N.D.=Not done.

FIG. 6

Confirmation that an intermolecular gp120-gp41 bond forms in the SOS gp140 protein. 293T cells were transfected with plasmids expressing gp140 proteins and, when indicated, a furin-expressing plasmid. The secreted, ³⁵S-labelled glycoproteins were immunoprecipitated with the indicated MAbs and analyzed by SDS-PAGE under reducing (+DTT) or nonreducing conditions. (a) Radioimmunoprecipitations with 2G12 of the SOS gp140, gp140WT and gp140UNC proteins. Immunoprecipitated proteins were resolved by SDS-PAGE under reducing (Lanes 4-6) or non-reducing (Lanes 1-3) conditions. (b) Radioimmunoprecipitations with 2G12 of the SOS gp140 protein and gp140 proteins containing the corresponding single-cysteine mutations. 140 kDa protein bands are not observed for either the A492C or the T596C single-cysteine mutant gp140 proteins. (c) Radioimmunoprecipitations with 2G12 of the SOS gp140 proteins produced in the presence or absence of co-transfected furin. Immunoprecipitated proteins were resolved by SDS-PAGE under reducing (Lanes 3-4) or non-reducing (Lanes 1-2) conditions. DTT is shown to reduce the 140 kDa SOS protein band produced in the presence but not the absence of exogenous furin.

FIG. 7

Analysis of cysteine mutants of JR-FL gp140. The ³⁵S-labelled envelope glycoproteins secreted from transfected 293T cells were immunoprecipitated with the anti-gp120 MAb 2G12, then analyzed by SDS-PAGE. All gp140s were expressed in the presence of co-transfected furin. Lanes 1-8, gp140s containing the indicated double cysteine mutations. Lanes 9-11, gp140 proteins containing the A492C/T596C double cysteine substitutions together with the indicated lysine to alanine substitutions at residue 491 (Lane 9), residue 493 (Lane 10) or at both residues 491 and 493 (Lane 11). Lanes 12-14, gp140 proteins containing quadruple cysteine substitutions.

FIG. 8

Comparison of the antigenic structures of the SOS gp140, W44C/T596C gp140 mutant, gp140UNC and gp140WT proteins. The ³⁵S-labelled envelope glycoproteins secreted from transfected 293T cells were immunoprecipitated with the indicated anti-gp120 Mabs and anti-gp41 MAbs, then analyzed by SDS-PAGE. Mutant but not wild type gp140s were expressed in the presence of cotransfected furin. (a) Anti-gp120 immunoglobulins that neutralize HIV-1_{JR-FL}. (b) Non-neutralizing antibodies to the C1, C4 and C5 regions of gp120 (c) Antibodies to CD4-induced epitopes were examined alone and in combination with sCD4. (d) Neutralizing (2F5) and non-neutralizing (7B2, 2.2B and 25C2) anti-gp41 antibodies and MAb 2G12. (e) Radioimmunoprecipitations of gp140WT (odd numbered lanes) and gp140UNC (even numbered lanes).

FIG. 9

Preparation of disulfide bond-stabilized gp140 proteins from various HIV-1 isolates. 293T cells were transfected with plasmids expressing wild type or mutant gp140s in the presence or absence of exogenous furin as indicated. ³⁵S-labeled supernatants were prepared and analyzed by radioimmunoprecipitation with MAb 2G12 as described above. Lane 1: SOS gp140 protein. Lane 2: gp140WT plus furin. Lane 3: gp140WT without furin. (a) HIV-1_{DH123}. (b) HIV-1_{HxB2}.

FIG. 10

Amino acid sequences of the glycoproteins with various deletions in the variable regions. The deleted wild-type sequences are shown in the white shade and include the following: ΔV1: D132-K152; ΔV2: F156-I191; ΔV1V2′: D132-K152 and F156-I191; ΔV1V2*: V126-S192; ΔV3: N296-Q324.

FIG. 11

Formation of an intersubunit cysteine bridge in envelope proteins with deletions in variable loop regions. (a) The ΔV1V2*V3 protein and the ΔV1V2*V3 N357Q N398Q protein with two cysteines at positions 492 and 596 (indicated with CC) were precipitated with 2G12 and F91 (Lanes 3 and 7, and 4 and 8, respectively). The appropriate controls without cysteine mutations are shown in Lanes 1, 2, 5, and 6. The wild-type protein without extra cysteines is shown in lanes 9 and 10. All the proteins were cleaved by furin, except for the wild-type protein of lane 10. The approximate sizes in kDa are given on the right. (b) Various loop deleted proteins with two cysteines at positions 492 and 596 (CC) were precipitated with 2G12 (Lanes 3, 5, 7, 9, 11, and 13). Proteins with the same deletions without extra cysteines are given in the adjacent lanes. These control proteins were not cleaved by furin. The full-length SOS gp140 protein is included as a control in Lane 1.

FIG. 12

Antigenic characterization of the A492C/T596C mutant in combination with deletions in the variable loops. All mutants were expressed in the presence of exogenous furin. The antibodies used in RIPAs are indicated on top. (a) The A492C/T596C ΔV1V2* mutant and (b) the A492C/T596C ΔV3 mutant.

FIG. 13

Nucleotide (a) and amino acid (b) sequences for HIV-1_{JR-FL} SOS gp140. The amino acid numbering system corresponds to that for wild-type JR-FL (Genbank Accession Number U63632). The cysteine mutations are indicated in underlined bold type face.

FIG. 14

Nucleotide (a) and amino acid (b) sequences for HIV-1_{JR-FL} ΔV1V2* SOS gp140. The amino acid numbering system corresponds to that for wild-type JR-FL (Genbank Accession Number U63632). The cysteine mutations are indicated in underlined bold type face.

FIG. 15

Nucleotide (a) and amino acid (b) sequences for HIV-1_{JR-FL} ΔV3 SOS gp140. The amino acid numbering system corresponds to that for wild-type JR-FL (Genbank Accession Number U63632) The cysteine mutations are indicated in underlined bold type face.

FIG. 16

SDS-PAGE analysis of purified HIV-1_{JR-FL} SOS gp140, gp140UNC and gp120 proteins. CHO cell-expressed proteins (0.5 μg) in Laemmli sample buffer with (reduced) or without (non-reduced) 50 mM DTT were resolved on a 3-8% polyacrylamide gradient gel.

FIG. 17

Biophysical analyses of purified, CHO cell-expressed HIV-1_{JR-FL} envelope glycoproteins. (a) Ultracentrifugation analysis of SOS gp140 was performed at protein concentrations ranging from 0.25 mM to 11.0 mM. The experimental data (open circles) were compared with theoretical curves for ideal monomers, dimers and trimers (labeled 1, 2, and 3). (b) Analytical size exclusion chromatography. Purified SOS gp140, gp140UNC and gp120 proteins were resolved on a TSK G3000SWXL column in PBS buffer, and their retention times were compared with those of known molecular weight standard proteins of 220 kDa, 440 kDa and 880 kDa (arrowed). The main peak retention time of SOS gp140 (5.95 minutes) is consistent with it being a monomer that is slightly larger than monomeric gp120 (retention time 6.24 minutes), whereas gp140UNC (retention time 4.91 minutes) migrates as oligomeric species. (c) The oligomeric status of pure standard proteins, thyroglobulin, ferritin and albumin, were compared with gp120 and gp120 in complex with soluble CD4 using BN-PAGE. The proteins were visualized on the gel using coomassie blue. (d) BN-PAGE analysis of CHO cell-derived, purified HIV-1_{JR-FL} gp120, SOS gp140 and gp140UNC glycoproteins.

FIG. 18

Negative stain electron micrographs of SOS gp140 alone (a) and in complex with MAbs (b-f). Bar=40 nm. In b-f, the panels were masked and rotated so that the presumptive Fc of the MAb is oriented downward. When multiple MAbs were used, the presumptive Fc of MAb 2F5 is oriented downward. In b-f, interpretative diagrams are also provided to illustrate the basic geometry and stoichiometry of the immune complexes. SOS gp140, intact MAb, and F(ab′)2 are illustrated by ovals, Y-shaped structures and V-shaped structures, respectively, in the schematic diagrams, which are not drawn to scale. The MAbs used are as follows: (b) 2F5; (c) IgG1b12; (d) 2G12; (e) MAb 2F5 plus F(ab′) 2 IgG1b12; (f) MAb 2F5 plus MAb 2G12.

FIG. 19

Individual, averaged and subtracted electron micrographs of SOS gp140 and gp120 in complex with sCD4 and MAb 17b. Bar=40 nm. Panels a and b are individual electron micrographs of ternary complexes of SOS gp140 (a) and YU2 gp120 (b). The Fc region of MAb 17b is aligned downward. Panels c and f are averaged electron micrographs of ternary complexes of SOS gp140 (Panel c) and gp120 (Panel f). Panels d and g are masked and averaged electron micrographs of the SOS gp140 complex (Panel d) and the gp120 complex (Panel g). Panel e represents the density remaining upon subtraction of the gp120 complex (Panel g) from the gp140 complex (Panel d). In Panels d and e, the arrow indicates the area of greatest residual density, which represents the presumptive gp41ECTO moiety that is present in SOS gp140 but not in gp120. Panel h indicates the outline of the gp120 complex (Panel g) overlaid upon a ribbon diagram of the X-ray crystal structure of the gp120 core in complex with sCD4 and the 17b Fab fragment [PDB code 1GC1] (Kwong, 1998). The gp120 complex was enlarged to facilitate viewing.

FIG. 20

Models indicating the approximate location of gp41ECTO in relation to gp120 as derived from electron microscopy data of SOS gp140. (a) Presumptive location of gp41ECTO (represented by the dark blue oval) in relation to the X-ray crystal structure of the gp120 core in complex with sCD4 (yellow) and Fab 17b (light blue) [PDB code 1GC1] (Kwong, 1998). The gp120 core surface was divided into three faces according to their antigenic properties (Moore, 1996; and Wyatt, 1998a); the non-neutralizing face is colored lavender, the neutralizing face is red, and the silent face, green. (b) The IgG1b12 epitope (Saphire, 2001) and the 2G12 epitope (Wyatt, 1998a) are shown in yellow and white, respectively. The residues associated with the gp120 C-terminus is colored blue, to provide a point of reference.

FIG. 21

RIPA analysis of unpurified, CHO cell-expressed HIV-1_{JR-FL} SOS gp140. Stably transfected CHO cells were cultured in the presence of ³⁵S-labeled cysteine and methionine. Culture supernatants were immunoprecipitated with the indicated MAbs and protein G-agarose beads, and bound proteins were resolved by SDS-PAGE and visualized by autoradiography. The MAb and/or CD4-based protein used for capture is indicated above each lane. In Lane 2, the proteins were reduced with DTT prior to SDS-PAGE; the remaining samples were analyzed under non-reducing conditions.

FIG. 22

SPR analysis of CHO cell-expressed HIV-1_{JP-FL} SOS gp140, gp140UNC and gp120 proteins. Anti-gp120 and anti-gp41 MAbs were immobilized onto sensor chips and exposed to buffers containing the indicated gp120 or gp140 glycoproteins in either purified or unpurified form, as indicated. Where noted, Env proteins were mixed with an 8-fold molar excess of sCD4 for 1 h prior to analysis. Culture supernatants from stably transfected CHO cells were used as the source of unpurified SOS gp140 and gp140UNC proteins. The concentrations of these proteins were measured by Western blotting and adjusted so that approximately equal amounts of each protein were loaded. Only the binding phases of the sensorgrams are shown; in general, the dissociation rates were too slow to provide meaningful information.

FIG. 23

BN-PAGE analyses of unfractionated cell culture supernatants. (a) Comparison of HIV-1_{JR-FL} gp120, SOS gp140, gp140UNC, and ΔV1V2 SOS gp140 glycoproteins present in culture supernatants from stable CHO cell lines. (b) Proteolytic cleavage destabilizes gp140 oligomers. 293T cells were transfected with furin and plasmids encoding SOS gp140, gp140UNC, SOS gp140UNC. Cell culture supernatants were combined with MOPS buffer containing 0.1% coomassie blue and resolved by BN-PAGE. Proteins were then transferred to PVDF membranes and visualized by Western blotting. Thyroglobulin and the BSA dimer were used as molecular weight markers (see FIG. 2c).

FIG. 24

HIV-1_{JR-FL} gp120 immobilization onto PA1-microbeads. HIV-1_{JR-FL} gp120 was immobilized onto PA1 magnetic microbeads as described. 5 μl and 12.5 μl of the resuspended beads were analyzed under reducing conditions on SDS-PAGE followed by Coomassie staining. 2.5 μg of gp120 was loaded for comparison and quantitation.

FIG. 25.

HIV-1_{JR-FL} gp120 immobilization onto PA1-Dynabeads. HIV-1_{JR-FL} gp120 was immobilized onto PA1 magnetic Dynabeads as described. Indicated volumes of the resuspended beads were analyzed under reducing conditions on SDS-PAGE followed by Coomassie staining. Increasing amounts of gp120 were loaded for quantitation.

FIG. 26

Temporal analysis of anti-gp120 antibody response elicited by gp120 vaccines. Serum response was analyzed after each immunization, using a native gp120-specific ELISA assay. Dose of gp120 is indicated in parentheses in legend.

FIG. 27

Anti-gp120 titers (50% maximal) in serum from animals immunized with three doses of gp120 vaccine. Data are mean +/− SD of 5 animals per group, and dose of gp120 is in parentheses.

### DETAILED DESCRIPTION OF THE INVENTION

This invention provides a first composition comprising a pharmaceutically acceptable particle and a stable HIV-1 pre-fusion envelope glycoprotein trimeric complex operably affixed thereto, each monomeric unit of the complex comprising HIV-1 gp120 and HIV-1 gp41, wherein (i) the gp120 and gp41 are bound to each other by at least one disulfide bond between a cysteine residue introduced into the gp120 and a cysteine residue introduced into the gp41, and (ii) the gp120 has deleted from it at least one V-loop present in wild-type HIV-1 gp120.

In one embodiment, the stable HIV-1 pre-fusion envelope glycoprotein trimeric complex is operably affixed to the particle via an agent which is operably affixed to the particle.

The first composition can further comprise a pharmaceutically acceptable carrier. The first composition can also further comprise an adjuvant.

In one embodiment, the gp120 has deleted from it one or more of variable loops V1, V2 and V3. In another embodiment, the disulfide bond is formed between a cysteine residue introduced by an A492C mutation in gp120 and a cysteine residue introduced by a T596C mutation in gp41. In a further embodiment, the gp120 is further characterized by (i) the absence of one or more canonical glycosylation sites present in wild-type HIV-1 gp120, and/or (ii) the presence of one or more canonical glycosylation sites absent in wild-type HIV-1 gp120.

The particle can be, for example, a paramagnetic bead, a non-paramagnetic bead, a liposome or any combination thereof. The particle can comprise, for example, PLG, latex, polystyrene, polymethyl-methacrylate, or any combination thereof.

As used herein, non-paramagnetic beads may contain, for example, metal oxides, aluminum phosphate, aluminum hydroxide, calcium phosphate, or calcium hydroxide.

In one embodiment, the mean diameter of the particle is from about 10 nm to 100 μm. In a further embodiment, the mean diameter of the particle is from about 10 nm to 10 μm. In a further embodiment, the mean diameter of the particle is from about 100 nm to 1 μm. In a further embodiment, the mean diameter of the particle is from about 1 μm to 10 μm. In a further embodiment, the mean diameter of the particle is from about 10 μm to 100 μm. In a further embodiment, the mean diameter of the particle is from about 10 nm to 100 nm. In a further embodiment, the mean diameter of the particle is about 50 nm.

In the first composition, wherein the agent can be, for example, an antibody, a fusion protein, streptavidin, avidin, a lectin, or a receptor. In one embodiment, the agent is CD4 or an antibody.

In the first composition, the adjuvant can be, for example, alum, Freund's incomplete adjuvant, saponin, Quil A, QS-21, Ribi Detox, monophosphoryl lipid A, a CpG oligonucleotide, CRL-1005, L-121, or any combination thereof.

The first composition can further comprise a cytokine and/or a chemokine. Cytokines include, for example, interleukin-2, interleukin-4, interleukin-5, interleukin-12, interleukin-15, interleukin-18, GM-CSF, and any combination thereof. Chemokines include, for example, SLC, ELC, Mip3α, Mip3β, IP-10, MIG, and any combination thereof.

Cytokines include but are not limited to interleukin-4, interleukin-5, interleukin-2, interleukin-12, interleukin-15, interleukin-18, GM-CSF, and combinations thereof.

Chemokines include but are not limited to SLC, ELC, Mip-3α, Mip-3β, interferon inducible protein 10 (IP-10), MIG, and combinations thereof.

This invention further provides a method for eliciting an immune response in a subject against HIV-1 or an HIV-1-infected cell comprising administering to the subject a prophylactically or therapeutically effective amount of the first composition. The composition can be administered in a single dose or in multiple doses.

In one embodiment, the first composition is administered as part of a heterologous prime-boost regimen.

This invention further provides a vaccine which comprises a therapeutically effective amount of the first composition and a pharmaceutically acceptable carrier.

This invention further provides a vaccine which comprises a prophylactically effective amount of the first composition and a pharmaceutically acceptable carrier.

This invention further provides a method for preventing a subject from becoming infected with HIV-1 comprising administering to the subject a prophylactically effective amount of the first composition, thereby preventing the subject from becoming infected with HIV-1.

This invention further provides a method for reducing the likelihood of a subject's becoming infected with HIV-1 comprising administering to the subject a prophylactically effective amount of the first composition, thereby reducing the likelihood of the subject's becoming infected with HIV-1.

In one embodiment of the instant methods, the subject is HIV-1-exposed.

This invention further provides a method for preventing, or delaying the onset of, or slowing the rate of progression of, an HIV-1-related disease in an HIV-1-infected subject which comprises administering to the subject a therapeutically effective amount of the first composition, thereby preventing or delaying the onset of, or slowing the rate of progression of, the HIV-1-related disease in the subject.

This invention further provides a method for producing the first composition, comprising contacting a pharmaceutically acceptable particle with a stable HIV-1 pre-fusion envelope glycoprotein trimeric complex under conditions permitting the complex to become operably affixed to the particle, wherein each monomeric unit of the complex comprises HIV-1 gp120 and HIV-1 gp41, (i) the gp120 and gp41 being bound to each other by at least one disulfide bond between a cysteine residue introduced into the gp120 and a cysteine residue introduced into the gp41, and (ii) the gp120 having deleted from it at least one V-loop present in wild-type HIV-1 gp120.

This invention further provides a second method for producing the first composition, comprising contacting (a) a pharmaceutically acceptable particle having operably affixed thereto an agent which binds to a stable HIV-1 pre-fusion envelope glycoprotein trimeric complex and (b) a stable HIV-1 pre-fusion envelope glycoprotein trimeric complex under conditions permitting the complex to bind to the agent, thereby permitting the complex to become operably affixed to the particle, wherein each monomeric unit of the complex comprises HIV-1 gp120 and HIV-1 gp41, (i) the gp120 and gp41 being bound to each other by at least one disulfide bond between a cysteine residue introduced into the gp120 and a cysteine residue introduced into the gp41, and (ii) the gp120 having deleted from it at least one V-loop present in wild-type HIV-1 gp120.

In one embodiment, the stable HIV-1 pre-fusion envelope glycoprotein trimeric complex of part (b) is present in a heterogeneous protein sample.

This invention further provides a method for isolating a stable HIV-1 pre-fusion envelope glycoprotein trimeric complex comprising contacting, under suitable conditions, a stable HIV-1 pre-fusion envelope glycoprotein trimeric complex-containing sample with a pharmaceutically acceptable particle having operably affixed thereto an agent which specifically binds to the trimeric complex, wherein each monomeric unit of the complex comprises HIV-1 gp120 and HIV-1 gp41, (i) the gp120 and gp41 being bound to each other by at least one disulfide bond between a cysteine residue introduced into the gp120 and a cysteine residue introduced into the gp41, and (ii) the gp120 having deleted from it at least one V-loop present in wild-type HIV-1 gp120; and separating the particle from the sample, thereby isolating the trimeric complex.

This invention further provides a second composition comprising (a) a pharmaceutically acceptable particle, (b) an antigen, and (c) an agent which is operably affixed to the particle and is specifically bound to the antigen, whereby the antigen is operably bound to the particle.

In one embodiment, the antigen is a tumor-associated antigen. In another embodiment, the antigen is derived from a pathogenic microorganism.

In one embodiment, the second composition further comprises a pharmaceutically acceptable carrier. In another embodiment, the second composition further comprises an adjuvant.

In the second composition, the particle is of the same material and dimensions, and the agent, adjuvant, cytokine and chemokine are of the same nature, as in the first composition.

This invention further provides a method for eliciting an immune response against an antigen in a subject comprising administering to the subject a prophylactically or therapeutically effective amount of the second composition, wherein the composition comprises the antigen against which the immune response is elicited operatively bound to the particle of the composition.

In the instant method, the composition can be administered in a single dose or in multiple doses. The composition can also be administered as part of a heterologous prime-boost regimen.

This invention also provides a vaccine which comprises a therapeutically effective amount of the second composition and a pharmaceutically acceptable carrier. This invention further provides a vaccine which comprises a prophylactically effective amount of the second composition and a pharmaceutically acceptable carrier.

This invention further provides a method for preventing a subject from becoming infected with a virus comprising administering to the subject a prophylactically effective amount of the second composition, wherein the antigen of the composition is present on the surface of the virus, thereby preventing the subject from becoming infected with the virus.

This invention further provides a method for reducing the likelihood of subject's becoming infected with a virus comprising administering to the subject a prophylactically effective amount of the second composition, wherein the antigen of the composition is present on the surface of the virus, thereby reducing the likelihood of the subject's becoming infected with the virus.

In one embodiment, the subject has been exposed to the virus.

This invention further provides a method for preventing or delaying the onset of, or slowing the rate of progression of, a virus-related disease in a virus-infected subject comprising administering to the subject a therapeutically effective amount of the second composition, wherein the antigen of the composition is present on the surface of the virus, thereby preventing or delaying the onset of, or slowing the rate of progression of, the virus-related disease in the subject.

This invention further provides a method for producing the second composition, comprising contacting (a) a pharmaceutically acceptable particle having operably affixed thereto an agent which specifically binds to an antigen and (b) the antigen, under conditions permitting the antigen to bind the agent, thereby permitting the antigen to become operably affixed to the particle.

The antigen can be, for example, a tumor-associated antigen or an antigen derived from a pathogenic microorganism.

This invention further provides a method for eliciting an immune response against a tumor-specific antigen in a subject comprising administering to the subject a prophylactically or therapeutically effective amount of the second, tumor-related composition.

This invention further provides a method for preventing the growth of, or slowing the rate of growth of, a tumor in a subject comprising administering to the subject a therapeutically effective amount of the second, tumor-related composition, wherein the tumor-associated antigen of the composition is present on the surface of cells of the tumor, thereby preventing the growth of, or slowing the rate of growth of, the tumor in the subject.

This invention further provides a method for reducing the size of a tumor in a subject comprising administering to the subject a therapeutically effective amount of the second, tumor-related composition, wherein the tumor-associated antigen of the composition is present on the surface of cells of the tumor, thereby reducing the size of the tumor in the subject.

Finally, this invention provides antibodies directed against the instant trimeric complex.

Set forth below are certain additional definitions and examples which are intended to aid in an understanding of the instant invention.

As used herein, “operably affixed”, when in reference to a trimeric complex or other antigen on a particle, means affixed so as to permit recognition of the complex or other antigen by an immune system. A “pharmaceutically acceptable particle” means any particle made of a material suitable for introduction into a subject.

As used herein, “subject” means any animal or artificially modified animal. Artificially modified animals include, but are not limited to, SCID mice with human immune systems. Animals include, but are not limited to, mice, rats, dogs, guinea pigs, ferrets, rabbits, and primates. In the preferred embodiment, the subject is a human.

As used herein, to “enhance the stability” of an entity, such as a protein, means to make the entity more long-lived or resistant to dissociation. Enhancing stability can be achieved, for example, by the introduction of disulfide bonds, salt bridges, hydrogen bonds, hydrophobic interactions, favorable van der Waals contacts, a linker peptide or a combination thereof. Stability enhancing changes can be introduced by recombinant methods.

As used herein, “HIV” shall mean the human immunodeficiency virus. HIV shall include, without limitation, HIV-1.

The human immunodeficiency virus (HIV) may be either of the two known types of HIV (HIV-1 or HIV-2). The HIV-1 virus may represent any of the known major subtypes (Classes A, B, C, D E, F, G and H) or outlying subtype (Group 0).

HIV-1_{JR-FL} is a strain that was originally isolated from the brain tissue of an AIDS patient taken at autopsy and co-cultured with lectin-activated normal human PBMCs (O'Brien, 1990). HIV-1_{JR-FL} is known to utilize CCR5 as a fusion coreceptor and has the ability to replicate in phytohemagglutinin (PHA)-stimulated PBMCs and blood-derived macrophages but does not replicate efficiently in most immortalized T cell lines.

HIV-1_{DH123} is a clone of a virus originally isolated from the peripheral mononuclear cells (PBMCs) of a pateint with AIDS (Shibata, 1995). HIV-1_{DH123} is known to utilize both CCR5 and CXCR4 as fusion coreceptors and has the ability to replicate in PHA-stimulated PBMCs, blood-derived macrophages and immortalized T cell lines.

HIV-1_{Gun-1} is a cloned virus originally isolated from the peripheral blood mononuclear cells of a hemophilia B patient with AIDS (Takeuchi, 1987). HIV-1_{Gun-1} is known to utilize both CCR5 and CXCR4 as fusion coreceptors and has the ability to replicate in PHA-stimulated PBMCs, blood-derived macrophages and immortalized T cell lines.

HIV-1_{89.6} is a cloned virus originally isolated from a patient with AIDS (Collman, 1992). HIV-1_{89.6} is known to utilize both CCR5 and CXCR4 as fusion coreceptors and has the ability to replicate in PHA-stimulated PBMCs, blood-derived macrophages and immortalized T cell lines.

HIV-1_{HXB2} is a TCLA virus that is known to utilize CXCR4 as a fusion coreceptor and has the ability to replicate in PHA-stimulated PBMCs and immortalized T cell lines but not blood derived macrophages.

Although the above strains are used herein to generate the mutant viral envelope proteins of the subject invention, other HIV-1 strains could be substituted in their place as is well known to those skilled in the art.

The human imunodeficiency virus includes but is not limited to the JR-FL strain. The surface protein includes but is not limited to gp120. An amino acid residue of the C1 region of gp120 may be mutated. An amino acid residue of the C5 region of gp120 may be mutated. The amino acids residues which may be mutated include but are not limited to the following amino acid residues: V35; Y39, W44; G462; 1482; P484; G486; A488; P489; A492; and E500. The gp120 amino acid residues are also set forth in FIG. 3a. The transmembrane protein includes but is not limited to gp41. An amino acid in the ectodomain of gp41 may be mutated. The amino acids residues which may be mutated include but are not limited to the following amino acid residues: D580; W587; T596; V599; and P600. The gp41 amino acid residues are also set forth in FIG. 3b.

As used herein, “HIV gp140 protein” shall mean a protein having two disulfide-linked polypeptide chains, the first chain comprising the amino acid sequence of the HIV gp120 glycoprotein and the second chain comprising the amino acid sequence of the water-soluble portion of HIV gp41 glycoprotein (“gp41 portion”). HIV gp140 protein includes, without limitation, proteins wherein the gp41 portion comprises a point mutation such as 1559G, L566V, T569P and 1559P. HIV gp140 protein comprising such mutations is also referred to as “HIV SOSgp140”, as well as “HIV gp140 monomer.”

In one embodiment, gp140 comprises gp120 or a modified form of gp120 which has modified immunogenicity relative to wild type gp120. In another embodiment, the modified gp120 molecule is characterized by the absence of one or more variable loops present in wild type gp120. In another embodiment, the variable loop comprises V1, V2, or V3. In another embodiment, the modified gp120 molecule is characterized by the absence or presence of one or more canonical glycosylation sites not present in wild type gp120. In another embodiment, one or more canonical glycosylation sites are absent from the V1V2 region of the gp120 molecule.

As used herein, “gp41” shall include, without limitation, (a) whole gp41 including the transmembrane and cytoplasmic domains; (b) gp41 ectodomain (gp41ECTO); (c) gp41 modified by deletion or insertion of one or more glycosylation sites; (d) gp41 modified so as to eliminate or mask the well-known immunodominant epitope; (e) a gp41 fusion protein; and (f) gp41 labeled with an affinity ligand or other detectable marker. As used herein, “ectodomain” means the extracellular region of a transmembrane protein exclusive of the transmembrane spanning and cytoplasmic regions.

Pharmaceutically acceptable carriers are well known to those skilled in the art and include, but are not limited to, 0.01-0.1M and preferably 0.05M phosphate buffer, phosphate buffered saline, or 0.9% saline. Additionally, such pharmaceutically acceptable carriers may include, but are not limited to, aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, chelating agents, inert gases and the like.

As used herein, “adjuvants” shall mean any agent suitable for enhancing the immunogenicity of an antigen such as protein and nucleic acid. Adjuvants suitable for use with protein-based vaccines include, but are not limited to, alum, Freund's incomplete adjuvant (FIA), Saponin, Quil A, QS21, Ribi Detox, Monophosphoryl lipid A (MPL), and nonionic block copolymers such as L-121 (Pluronic; Syntex SAF). In a preferred embodiment, the adjuvant is alum, especially in the form of a thixotropic, viscous, and homogenous aluminum hydroxide gel. The vaccines of the subject invention may be administered as an oil-in-water emulsion. Methods of combining adjuvants with antigens are well known to those skilled in the art.

Adjuvants may also be in particulate form. The antigen may be incorporated into biodegradable particles composed of poly-lactide-co-glycolide (PLG) or similar polymeric material. Such biodegradable particles are known to provide sustained release of the immunogen and thereby stimulate long-lasting immune responses to the immunogen. Other particulate adjuvants, include but are not limited to, micellular mixtures of Quil A and cholesterol known as immunostimulating complexes (ISCOMs) and aluminum or iron oxide beads. Methods for combining antigens and particulate adjuvants are well known to those skilled in the art. It is also known to those skilled in the art that cytotoxic T lymphocyte and other cellular immune responses are elicited when protein-based immunogens are formulated and administered with appropriate adjuvants, such as ISCOMs and micron-sized polymeric or metal oxide particles.

Suitable adjuvants for nucleic acid based vaccines include, but are not limited to, Quil A, interleukin-12 delivered in purified protein or nucleic acid form, short bacterial immunostimulatory nucleotide sequence such as CpG-containing motifs, interleukin-2/Ig fusion proteins delivered in purified protein or nucleic acid form, oil in water micro-emulsions such as MF59, polymeric microparticles, cationic liposomes, monophosphoryl lipid A (MPL), immunomodulators such as Ubenimex, and genetically detoxified toxins such as E. coli heat labile toxin and cholera toxin from Vibrio. Such adjuvants and methods of combining adjuvants with antigens are well known to those skilled in the art.

As used herein, “A492C mutation” refers to a point mutation of amino acid 492 in HIV-1_{JR-FL} gp120 from alanine to cysteine. Because of the sequence variability of HIV, this amino acid will not be at position 492 in all other HIV isolates. For example, in HIV-_{1NL4-3} the corresponding amino acid is A499 (Genbank Accession Number AAA44992). It may also be a homologous amino acid other than alanine or cysteine. This invention encompasses cysteine mutations in such amino acids, which can be readily identified in other HIV isolates by those skilled in the art.

As used herein, “T596C mutation” refers to a point mutation of amino acid 596 in HIV-1_{JR-FL} gp41 from threonine to cysteine. Because of the sequence variability of HIV, this amino acid will not be at position 596 in all other HIV isolates. For example, in HIV-1_{NL4-3} the corresponding amino acid is T603 (Genbank Accesion Number AAA44992). It may also be a homologous amino acid other than threonine or cysteine. This invention encompasses cysteine mutations in such amino acids, which can be readily identified in other HIV isolates by those skilled in the art.

As used herein, “canonical glycosylation site” includes but is not limited to an Asn-X-Ser or Asn-X-Thr sequence of amino acids that defines a site for N-linkage of a carbohydrate. In addition, Ser or Thr residues not present in such sequences to which a carbohydrate can be linked through an O-linkage are canonical glycosylation sites. In the later case of a canonical glycosylation site, a mutation of the Ser and Thr residue to an amino acid other than a serine or threonine will remove the site of O-linked glycosylation.

As used herein, “C1 region” means the first conserved sequence of amino acids in the mature gp120 glycoprotein. The C1 region includes the amino-terminal amino acids. In HIV-1_{JR-FL}, the C1 region consists of the amino acids VEKLWVTVYYGVPVWKEATTTLFCASDAKAYDTEVHNVWATHACVPTDPNPQEVVLENVT EHFNMWKNNMVEQMQEDIISLWDQSLKPCVKLTPLCVTLN. Amino acid resides 30-130 of the sequence set forth in FIG. 3a have this sequence. In other HIV isolates, the C1 region will comprise a homologous amino-terminal sequence of amino acids of similar length. W44C and P600C mutations are as defined above for A492 and T596 mutations. Because of the sequence variability of HIV, W44 and P600 will not be at positions 44 and 600 in all HIV isolates. In other HIV isolates, homologous, non-cysteine amino acids may also be present in the place of the tryptophan and proline. This invention encompasses cysteine mutations in such amino acids, which can be readily identified in other HIV isolates by those skilled in the art.

As used herein, “C5 region” means the fifth conserved sequence of amino acids in the gp120 glycoprotein. The C5 region includes the carboxy-terminal amino acids. In HIV-1_{JR-FL} gp120, the unmodified C5 region consists of the amino acids GGGDMRDNWRSELYKYKVVKIEPLGVAPTKAKRRVVQRE. Amino acid residues 462-500 of the sequence set forth in FIG. 3a have this sequence. In other HIV isolates, the C5 region will comprise a homologous carboxy-terminal sequence of amino acids of similar length.

As used herein, non-paramagnetic beads may contain, for example, metal oxides, aluminum phosphate, aluminum hydroxide, calcium phosphate, or calcium hydroxide.

Cytokines and chemokines can be provided to a subject via a vector expressing one or more cytokines.

As used herein “prophylactically effective amount” means amount sufficient to reduce the likelihood of a disorder from occurring.

As used herein, “therapeutically effective amount” means an amount effective to slow, stop or reverse the progression of a disorder.

As used herein, “virally infected” means the introduction of viral genetic information into a target cell, such as by fusion of the target cell membrane with the virus or infected cell. The target may be a cell of a subject. In the preferred embodiment, the target cell is a cell in a human subject.

This invention provides a vaccine which comprises the above isolated nucleic acid. In one embodiment, the vaccine comprises a therapeutically effective amount of the nucleic acid. In another embodiment, the vaccine comprises a therapeutically effective amount of the protein encoded by the above nucleic acid. In another embodiment, the vaccine comprises a combination of the recombinant nucleic acid molecule and the mutant viral envelope protein.

In the instant vaccine, the vaccine can comprise, for example, a recombinant subunit protein, a DNA plasmid, an RNA molecule, a replicating viral vector, a non-replicating viral vector, or a combination thereof.

As used herein, “mutant” means that which is not wild-type.

As used herein, “immunizing” means generating an immune response to an antigen in a subject. This can be accomplished, for example, by administering a primary dose of a vaccine to a subject, followed after a suitable period of time by one or more subsequent administrations of the vaccine, so as to generate in the subject an immune response against the vaccine. A suitable period of time between administrations of the vaccine may readily be determined by one skilled in the art, and is usually on the order of several weeks to months.

The potential exists not only to substantially boost immune responses to the recombinant antigen, but to tailor the nature of the immune responses by priming and then delivering one or more subsequent boosts with different forms of the antigen or by delivering the antigen to different immunological sites and/or antigen-presenting cell populations. Indeed, the ability to induce preferred type-1 or type-2 like T-helper responses or to additionally generate specific responses at mucosal and/or systemic sites are envisioned with such an approach. Such protocols, also known as “Prime-boost” protocols, are described in U.S. Pat. No. 6,210,663 B1 and WO 00/44410.

Examples of Prime Boost Regimens.

| Priming Composition | Boosting Composition |
|---|---|
| NA | AG |
| NA | AGP |
| NA | AG + AGP |
| AG | NA |
| AGP | NA |
| AG + AGP | NA |
| NA + AG | AGP |
| NA + AG | AG + AGP |
| NA + AG | AGP + NA |
| NA + AG + AGP | NA |
| NA + AG + AGP | NA + AG |
| NA + AG + AGP | NA + AGP |
| NA + AG + AGP | AG |
| NA + AG + AGP | AGP |
| NA + AG + AGP | AG + AGP |
| AG | NA |
| AGP | NA |
| AG + AGP | NA |
| AGP | NA + AG |
| AG + AGP | NA + AG |
| AGP + NA | NA + AG |
| NA | NA + AG + AGP |
| NA + AG | NA + AG + AGP |
| NA + AGP | NA + AG + AGP |
| AG | NA + AG + AGP |
| AGP | NA + AG + AGP |
| AG + AGP | NA + AG + AGP |
| AG | AGP |
| AG + AGP | AGP |
| AGP | AG |
| AGP | AG + AGP |

NA = Nucleic acid*
AG = Antigen
AGP = Particle-bound antigen
*The nucleic acid component in the above examples can be in the form of a viral vector component. The viral vector can be replicating-or non-replicating.

In one embodiment, vaccination is provided with at least three different vaccine compositions, wherein the vaccine compositions differ from each other by the form of the vaccine antigen.

For example, one embodiment of a priming vaccine composition is a replication-competent or replication-defective recombinant virus containing a nucleic acid molecule encoding the antigen, or a viral-like particle. In one particular embodiment, the priming composition is a non-replicating recombinant virus or viral-like particle derived from an α-virus.

One method according to this invention involves “priming” a mammalian subject by administration of a priming vaccine composition. “Priming”, as used herein, means any method whereby a first immunization using an antigen permits the generation of an immune response to the antigen upon a second immunization with the same antigen, wherein the second immune response is greater than that achieved where the first immunization is not provided.

In one embodiment, the priming vaccine, as with other instant compositions, is administered systemically. This systemic administration includes, for example, any parenteral route of administration characterized by physical breaching of a tissue of a subject and administration of an agent through the breach in the tissue. In particular, parenteral administration is contemplated to include, but is not limited to, intradermal, transdermal, subcutaneous, intraperitoneal, intravenous, intraarterial, intramuscular and intrasternal injection, intravenous, interaarterial and kidney dialytic infusion techniques, and so-called “needleless” injections through tissue. Preferably, the systemic, parenteral administration is intramauscular injection. In another embodiment, the instant vaccine is administered at a site of administration including the intranasal, oral, vaginal, intratracheal, intestinal and rectal mucosal surfaces.

The priming vaccine, as with other instant compositions, may be administered at various sites in the body in a dose-dependent manner. The invention is not limited to the amount or sites of injection(s) or to the pharmaceutical carrier, nor to this immunization protocol. Rather, the priming step encompasses treatment regimens which include a single dose or dosage which is administered hourly, daily, weekly, or monthly, or yearly.

“Priming amount” as used herein, means the amount of priming vaccine used.

Preferably, a boosting vaccine composition is administered about 2 to 27 weeks after administering the priming vaccine to a mammalian subject. The administration of the boosting vaccine is accomplished using an effective amount of a boosting vaccine containing or capable of delivering the same antigen as administered by the priming vaccine.

As used herein, the term “boosting vaccine” includes, as one embodiment, a composition containing the same antigen as in the priming vaccine or precursor thereof, but in a different form, in which the boosting vaccine induces an immune response in the host. In one particular embodiment, the boosting vaccine comprises a recombinant soluble protein.

In another example, one embodiment of a boosting vaccine composition is a replication-competent or replication-defective recombinant virus containing the DNA sequence encoding the protein antigen. In another embodiment, the boosting vaccine is a non-replicating α-virus comprising a nucleic acid molecule encoding the protein antigen or a non-replicating vaccine replicon particle derived from an Alphavirus. Adenoviruses, which naturally invade their host through the airways, infect cells of the airways readily upon intranasal application and induce a strong immune response without the need for adjuvants. In another embodiment, the boosting vaccine comprises a replication-defective recombinant adenovirus.

Another example of a boosting vaccine is a bacterial recombinant vector containing the DNA sequence encoding the antigen in operable association with regulatory sequences directing expression of the antigen in tissues of the mammal. One example is a recombinant BCG vector. Other examples include recombinant bacterial vectors based on Salmonella, Shigella, and Listeria, among others.

Still another example of a boosting vaccine is a naked DNA sequence encoding the antigen in operable association with regulatory sequences directing expression of the antigen in tissues of the mammal but containing no additional vector sequences. These vaccines may further contain pharmaceutically suitable or physiologically acceptable carriers.

In still additional embodiments, the boosting vaccines can include proteins or peptides (intact and denatured), heat-killed recombinant vaccines, inactivated whole microorganisms, antigen-presenting cells pulsed with the instant proteins or infected/transfected with a nucleic acid molecule encoding same, and the like, all with or without adjuvants, chemokines and/or cytokines.

Cytokines that may be used in the prime and/or boost vaccine or administered separately from the prime and/or boost vaccine include, but are not limited, to interleukin-4, interleukin-5, interleukin-2, interleukin-12, interleukin-15, interleukin-18, GM-CSF, and combinations thereof. The cytokine may be provided by a vector expressing one or more cytokines.

Representative forms of antigens include a “naked” DNA plasmid, a “naked” RNA molecule, a DNA molecule packaged into a replicating or nonreplicating viral vector, an RNA molecule packaged into a replicating or nonreplicating viral vector, a DNA molecule packaged into a bacterial vector, or proteinaceous forms of the antigen alone or present in virus-like particles, or combinations thereof.

As used herein, “virus-like particles” or VLPs are particles which are non-infectious in any host, nonreplicating in any host, which do not contain all of the protein components of live virus particles. In one embodiment, VLPs contain the instant trimeric and a structural protein, such as HIV-1 gag, needed to form membrane-enveloped virus-like particles.

Advantages of VLPs include (1) their particulate and multivalent nature, which is immunostimulatory, and (2) their ability to present the disulfide-stabilized envelope glycoproteins in a near-native, membrane-associated form.

VLPs are produced by co-expressing the viral proteins (e.g., HIV-1 gp120/gp41 and gag) in the same cell. This can be achieved by any of several means of heterologous gene expression that are well-known to those skilled in the art, such as transfection of appropriate expression vector(s) encoding the viral proteins, infection of cells with one or more recombinant viruses (e.g., vaccinia) that encode the VLP proteins, or retroviral transduction of the cells. A combination of such approaches can also be used. The VLPs can be produced either in vitro or in vivo.

VLPs can be produced in purified form by methods that are well-known to the skilled artisan, including centrifugation, as on sucrose or other layering substance, and by chromatography.

In one embodiment the instant nucleic acid delivery vehicle replicates in a cell of an animal or human being vaccinated. In one embodiment, said replicating nucleic acid has as least a limited capacity to spread to other cells of the host and start a new cycle of replication and antigen presentation and/or perform an adjuvant function. In another embodiment, the nucleic acid is non-replicating in an animal or human being being vaccinated. The nucleic acid can comprise nucleic acid of a poxvirus, a Herpes virus, a lentivirus, an Adenovirus, or adeno-associated virus. In a preferred embodiment, the nucleic acid comprises nucleic acid of an α-virus including, but not limited to, Venezuelan equine encephalitis (VEE) virus, Semliki Forest Virus, Sindbis virus, and the like. In another embodiment, said nucleic acid delivery vehicle is a VEE virus particle, Semliki Forest Virus particle, a Sindbis virus particle, a pox virus particle, a herpes virus particle, a lentivirus particle, or an adenovirus particle.

Depending on the nature of the vaccine and size of the subject, the dose of the vaccine can range from about 1 μg to about 1 mg. The preferred dose is about 300 μg.

In one aspect of the invention, vaccination is to be performed in a manner that biases the immune system in a preferred direction, for example, in the direction of a preferred T helper 1 type of immune response or a more T helper 2 type of immune response. It is now widely accepted that T cell-dependent immune responses can be classified on the basis of preferential activation and proliferation of two distinct subsets of CD4+ T-cells termed T_{H}1 and T_{H}2. These subsets can be distinguished from each other by restricted cytokine secretion profiles. The T_{H}1 subset is a high producer of IFN-γ with limited or no production of IL-4, whereas the T_{H}² phenotype typically shows high level production of both IL-4 and IL-5 with no substantial production of IFN-γ. Both phenotypes can develop from naïve CD4+ T cells and at present there is much evidence indicating that IL-12 and IFN-γ on the one hand and IL-4 on the other are key stimulatory cytokines in the differentiation process of pluripotent T_{H}0 precursor cells into T_{H}1 or T_{H}2 effector cells, respectively, in vitro and in vivo. Since IFN-γ inhibits the expansion and function of T_{H}2 effector cells and IL-4 has the opposite effect, the preferential expansion of either IFN-γ producing cells (pc) or IL-4 pc is indicative of whether an immune response mounts into a T_{H}1 or T_{H}2 direction. The cytokine environment, however, is not the only factor driving T_{H} lineage differentiation. Genetic background, antigen dose, route of antigen administration, type of antigen presenting cell (APC) and signaling via TCR and accessory molecules on T cells also play a role in differentiation.

In one aspect of the invention, the immune system is directed toward a more T helper 1 or 2 type of immune response through using vaccine compositions with the property of modulating an immune response in one direction or the other. In a preferred aspect of the invention at least part of said adjuvant function comprises means for directing the immune system toward a more T helper 1 or 2 type of immune response.

In another embodiment, the biasing is accomplished using vectors with the property of modulating an immune response in one direction or the other. Examples of vectors with the capacity to stimulate either a more T helper 1 or a more T helper 2 type of immune response or of delivery routes such as intramuscular or epidermal delivery can be found in Robinson, 1997; Sjolander, 1997; Doe, 1996; Feltquate, 1997; Pertmer, 1996; Prayaga, 1997; and Raz, 1996.

In another aspect of the invention, the immune system is induced to produce innate immune responses with adjuvant potential in the ability to induce local inflammatory responses. These responses include interferons, B-chemokines, and chemokines in general, capable of attracting antigen processing and presenting cells as well as certain lymphocyte populations for the production of additional specific immune responses. These innate type responses have different characteristics depending on the vector or DNA used and their specific immunomodulating characteristics, including those encoded by CpG motifs, and as such, the site of immunization. By using in a specific sequence vaccine compositions containing at least one common specific vaccine antigen, different kinds of desired protective vaccine responses may be generated and optimized. Different kinds of desired immune responses may also be obtained by combining different vaccine compositions and delivering them at different or the same specific sites depends on the desired vaccine effect at a particular site of entry (i.e. oral, nasal, enteric or urogenital) of the specific infectious agent.

In one aspect, the instant vaccine comprises antigen-presenting cells. Antigen-presenting cells include, but are not limited to, dendritic cells, Langerhan cell, monocytes, macrophages, muscle cells and the like. Preferably said antigen-presenting cells are dendritic cells. Preferably, said antigen-presenting cells present said antigen, or an immunogenic part thereof, such as a peptide, or derivative and/or analogue thereof, in the context of major histocompatibility complex I or complex II.

As used herein, “reducing the likelihood of a subject's becoming infected with a virus” means reducing the likelihood of the subject's becoming infected with the virus by at least two-fold. For example, if a subject has a 1% chance of becoming infected with the virus, a two-fold reduction in the likelihood of the subject becoming infected with the virus would result in the subject having a 0.5% chance of becoming infected with the virus. In the preferred embodiment of this invention, reducing the likelihood of the subject's becoming infected with the virus means reducing the likelihood of the subject's becoming infected with the virus by at least ten-fold.

As used herein, “exposured” to HIV-1 means contact with HIV-1 such that infection could result.

As used herein, “antigens” encompass, for example, monomeric proteins, multimeric proteins, glycoproteins, peptides and proteoglycans. The antigen may also be a membrane-bound protein. The antigen may also be a saccharide, oligosaccharide, glycolipid, or ganglioside. The antigen may be a virus or virus-like particle, or subfraction thereof. The antigen may be a bacterium, yeast, fungi or other infectious agent, or subfraction thereof.

An antigen may further be cell associated, derived or isolated from pathogenic microorganisms such as viruses including HIV, influenza, Herpes simplex, human papilloma virus (U.S. Pat. No. 5,719,054), Hepatitis B (U.S. Pat. No. 5,780,036), Hepatitis C (U.S. Pat. No. 5,709,995), EBV, Cytomegalovirus (CMV), RSV, West Nile Virus and the like.

An antigen may also be cell associated, derived or isolated from pathogenic bacteria or yeast such as from Chlamydia (U.S. Pat. No. 5,869,608), Mycobacteria, Legionella, Meningiococcus, Group A Streptococcus, Salmonella, Listeria, Hemophilus influenzae (U.S. Pat. No. 5,955,596), Aspergillus, invasive Candida (U.S. Pat. No. 5,645,992), Norcardia, Histoplasmosis, Cryptosporidia, and the like.

An antigen may also be cell associated, derived or isolated from a pathogenic protozoan or pathogenic parasite including but not limited to Pneumocystis carinii, Trypanosoma, Leishmania (U.S. Pat. No. 5,965,242), Plasmodium (U.S. Pat. No. 5,589,343) and Toxoplasma gondii.

An antigen may also be a polysaccharide or oligosaccharide derived from a capsular polysaccharide of a pathogenic bacterium or yeast, or a synthetic polysaccharide or oligosaccharide. Such capsular polysaccharides include but are not limited to capsular polysaccharide from Neisseria meningitidis serogroups A, C, W-135 and Y; pneumococcal polysaccharide from Streptococcus pneumoniae in particular serotype 1, 4, 5, 6B, 9V, 14, 18C, 19F, and 23F; Klebsiella capsular polysaccharide; Crytococcus neoformans capsular polysaccharide; Vi capsular polysaccharide of Salmonella typhi; and the like. Polysaccharide from one serotype or a multiplicity of serotypes may be utilized with the beads.

As used herein “tumor-associated antigens” (TAA) include, for example, an antigen associated with a preneoplastic or a hyperplastic state. The antigen may also be associated with, or causative of cancer. Such antigen may be a tumor cell, tumor specific antigen, tumor associated antigen or tissue specific antigen, epitope thereof, and epitope agonist thereof. Such antigens include but are not limited to carcinoembryonic antigen (CEA) and epitopes thereof such as CAP-1, CAP-1-6D, and the like (GenBank Accession Number. M29540), MART-1 (Kawakami, 1994a), MAGE-1 (U.S. Pat. No. 5,750,395), MAGE-3, GAGE (U.S. Pat. No. 5,648,226), GP-100 (Kawakami, 1994b), MUC-1, MUC-2, point mutated ras oncogene, normal and point mutated p53 oncogenes (Hollstein, 1994), PSMA (U.S. Pat. No. 5,538,866; Israeli, 1993), tyrosinase (Kwon, 1987), TRP-1 (gp75) (Chen, 1997), TRP-2 (Jackson, 1992), TAG72, KSA, CA-125, PSA, HER-2/neu/c-erb/B2 (U.S. Pat. No. 5,550,214), brc-I, brc-II, bcr-abl, pax3-fkhr, ews-fli-1, modifications of TAAs and tissue specific antigen, splice variants of TAAs, epitope aganists, and the like. Other TAAs may be identified, isolated and cloned by methods known in the art such as those disclosed in U.S. Pat. No. 4,514,506. The antigen may be encoded by a nucleic acid, such as a DNA plasmid, RNA molecule, or viral vector.

As used herein, “exposed” to the virus means contact with a virus such that infection could result.

A complete response in a patient with a tumor is defined as the disappearance of all clinical evidence of disease that lasts at least four weeks. A partial response is a 50% or greater decrease in the sum of the products of the perpendicular diameters the tumor for at least four weeks with no appearance of new tumors. Minor responses are defined as 25-49% decrease in the sum of the products of the perpendicular diameters of all measurable tumors with no appearance of new tumors and no size increase in any tumors.

Any patient with less than a partial response is considered a non-responder. The appearance of new tumors or greater than 25% increase in the product of perpendicular diameters of prior tumors following a partial or complete response is considered as a relapse.

Other measurable parameters of efficacy of treatment may include one or more of the following: (a) stabilization or decrease in serum PSA levels (for prostate cancer); (b) prolonged survival in comparison to subjects not treated with the composition; (c) prevention/inhibition of metastasis; and (d) immunological parameters such as increase in specific T cell mediated cytotoxicity, increase in cytokine production, increase in specific antibody responses.

The tumor-associated antigen of the present invention can form part of, or be derived from, cancers including but not limited to primary or metastatic melanoma, thymoma, lymphoma, sarcoma, lung cancer, liver cancer, non-Hodgkin's lymphoma, Hodgkins lymphoma, leukemias, uterine cancer, cervical cancer, bladder cancer, kidney cancer and adenocarcinomas such as breast cancer, prostate cancer, ovarian cancer, pancreatic cancer, and the like.

As used herein, the following standard abbreviations are used throughout the specification to indicate specific amino acids: A=ala=alanine; R=arg=arginine; N=asn=asparagine; D=asp=aspartic acid; C=cys=cysteine; Q=gln=glutamine; E=glu=glutamic acid; G=gly=glycine; H=his=histidine; I=ile=isoleucine; L=leu=leucine; K=lys=lysine; M=met=methionine; F=phe=phenylalanine; P=pro=proline; S=ser=serine; T=thr=threonine; W=trp=tryptophan; Y=tyr=tyrosine; V=val=valine; B=asx=asparagine or aspartic acid; Z=glx=glutamine or glutamic acid.

As used herein, the term “nucleic acid” shall mean any nucleic acid including, without limitation, DNA, RNA and hybrids thereof. The nucleic acid bases that form nucleic acid molecules can be the bases A, C, T, G and U, as well as derivatives thereof. Derivatives of these bases are well known in the art and are exemplified in PCR Systems, Reagents and Consumables (Perkin-Elmer Catalogue 1996-1997, Roche Molecular Systems, Inc, Branchburg, N.J., USA).

As used herein, the following standard abbreviations are used throughout the specification to indicate specific nucleotides: C=cytosine; A=adenosine; T=thymidine; G=guanosine; and U=uracil.

As used herein, “CCR5” is a chemokine receptor which binds members of the C—C group of chemokines and whose amino acid sequence comprises that provided in Genbank Accession Number 1705896 and related polymorphic variants. As used herein, CCR5 includes extracellular portions of CCR5 capable of binding the HIV-1 envelope protein.

As used herein, “CXCR4” is a chemokine receptor which binds members of the C—X—C group of chemokines and whose amino acid sequence comprises that provided in Genbank Accession Number 400654 and related polymorphic variants. As used herein, CXCR4 includes extracellular portions of CXCR4 capable of binding the HIV-1 envelope protein.

As used herein, “CDR” or complementarity determining region means a highly variable sequence of amino acids in the variable domain of an antibody. As used herein, a “derivatized” antibody is one that has been modified. Methods of derivatization include, but are not limited to, the addition of a fluorescent moiety, a radionuclide, a toxin, an enzyme or an affinity ligand such as biotin.

As used herein, “humanized” describes antibodies wherein some, most or all of the amino acids outside the CDR regions are replaced with corresponding amino acids derived from human immunoglobulin molecules. In one embodiment of the humanized forms of the antibodies, some, most or all of the amino acids outside the CDR regions have been replaced with amino acids from human immunoglobulin molecules but where some, most or all amino acids within one or more CDR regions are unchanged. Small additions, deletions, insertions, substitutions or modifications of amino acids are permissible as long as they do not abrogate the ability of the antibody to bind a given antigen. Suitable human immunoglobulin molecules include IgG1, IgG2, IgG3, IgG4, IgA, IgE and IgM molecules. A “humanized” antibody would retain an antigenic specificity similar to that of the original antibody.

One skilled in the art would know how to make the humanized antibodies of the subject invention. Various publications, several of which are hereby incorporated by reference into this application, also describe how to make humanized antibodies. For example, the methods described in U.S. Pat. No. 4,816,567 comprise the production of chimeric antibodies having a variable region of one antibody and a constant region of another antibody.

U.S. Pat. No. 5,225,539 describes another approach for the production of a humanized antibody. This patent describes the use of recombinant DNA technology to produce a humanized antibody wherein the CDRs of a variable region of one immunoglobulin are replaced with the CDRs from an immunoglobulin with a different specificity such that the humanized antibody would recognize the desired target but would not be recognized in a significant way by the human subject's immune system. Specifically, site directed mutagenesis is used to graft the CDRs onto the framework.

Other approaches for humanizing an antibody are described in U.S. Pat. Nos. 5,585,089 and 5,693,761 and WO 90/07861 which describe methods for producing humanized immunoglobulins. These have one or more CDRs and possible additional amino acids from a donor immunoglobulin and a framework region from an accepting human immunoglobulin. These patents describe a method to increase the affinity of an antibody for the desired antigen. Some amino acids in the framework are chosen to be the same as the amino acids at those positions in the donor rather than in the acceptor. Specifically, these patents describe the preparation of a humanized antibody that binds to a receptor by combining the CDRs of a mouse monoclonal antibody with human immunoglobulin framework and constant regions. Human framework regions can be chosen to maximize homology with the mouse sequence. A computer model can be used to identify amino acids in the framework region which are likely to interact with the CDRs or the specific antigen and then mouse amino acids can be used at these positions to create the humanized antibody.

The above U.S. Pat. Nos. 5,585,089 and 5,693,761, and WO 90/07861 also propose four possible criteria which may be used in designing the humanized antibodies. The first proposal was that for an acceptor, use a framework from a particular human immunoglobulin that is unusually homologous to the donor immunoglobulin to be humanized, or use a consensus framework from many human antibodies. The second proposal was that if an amino acid in the framework of the human immunoglobulin is unusual and the donor amino acid at that position is typical for human sequences, then the donor amino acid rather than the acceptor may be selected. The third proposal was that in the positions immediately adjacent to the 3 CDRs in the humanized immunoglobulin chain, the donor amino acid rather than the acceptor amino acid may be selected. The fourth proposal was to use the donor amino acid reside at the framework positions at which the amino acid is predicted to have a side chain atom within 3 Å of the CDRs in a three dimensional model of the antibody and is predicted to be capable of interacting with the CDRs. The above methods are merely illustrative of some of the methods that one skilled in the art could employ to make humanized antibodies.

One method for determining whether a subject has produced antibodies capable of blocking the infectivity of a virus is a diagnostic test examining the ability of the antibodies to bind to the stabilized viral envelope protein. As shown herein, such binding is indicative of the antibodies' ability to neutralize the virus. In contrast, binding of antibodies to non-stabilized, monomeric forms of viral envelope proteins is not predictive of the antibodies' ability to bind and block the infectivity of infectious virus (Fouts et al., J. Virol. 71:2779, 1997). The method offers the practical advantage of circumventing the need to use infectious virus.

Numerous immunoassay formats that are known to the skilled artisan are appropriate for this diagnostic application. For example, an enzyme-linked immunosorbent assay (ELISA) format could be used wherein in the mutant virus envelope glycoprotein is directly or biospecifically captured onto the well of a microtiter plate. After wash and/or blocking steps as needed, test samples are added to the plate in a range of concentrations. The antibodies can be added in a variety of forms, including but not limited to serum, plasma, and a purified immunoglobulin fraction. Following suitable incubation and wash steps, bound antibodies can be detected, such as by the addition of an enzyme-linked reporter antibody that is specific for the subject's antibodies. Suitable enzymes include horse radish peroxidase and alkaline phosphatase, for which numerous immunoconjugates and calorimetric substrates are commercially available. The binding of the test antibodies can be compared with that of a known monoclonal or polyclonal antibody standard assayed in parallel. In this example, high level antibody binding would indicate high neutralizing activity.

As an example, the diagnostic test could be used to determine if a vaccine elicited a protective antibody response in a subject, the presence of a protective response indicating that the subject was successfully immunized and the lack of such response suggesting that further immunizations are necessary.

Methods and conditions for purifying mutant envelope proteins from the culture media are provided in the invention, but it should be recognized that these procedures can be varied or optimized as is well known to those skilled in the art.

This invention will be better understood from the Examples that follow. However, one skilled in the art will readily appreciate that the specific methods and results discussed are merely illustrative of the invention as described more fully in the claims which follow thereafter.

### EXPERIMENTAL DETAILS

### Experimental Set I

A. Materials and Methods

The plasmid designated PPI4-tPA-gp120JR-FL was deposited pursuant to, and in satisfaction of, the requirements of the Budapest Treaty on the International Recognition of the Deposit of Microorganisms for the Purposes of Patent Procedure with the American Type Culture Collection (ATCC), 12301 Parklawn Drive, Rockville, Md. 20852 under ATCC Accession Number 75431. The plasmid was deposited with ATCC on Mar. 12, 1993. This eukaryotic shuttle vector contains the cytomegalovirus major immediate-early (CMV/MIE) promoter/enhancer linked to the full-length HIV-1 envelope gene whose signal sequence was replaced with that derived from tissue plasminogen activator. In the vector, a stop codon has been placed at the gp120 C-terminus to prevent translation of gp41 sequences, which are present in the vector. The vector also contains an ampicillin resistance gene, an SV40 origin of replication and a DHFR gene whose transcription is driven by the β-globin promoter.

The epitopes for, and some immunochemical properties of, anti-gp120 Mabs from various donors have been described previously (Moore, 1994a; and Moore, 1996). These include Mab 19b to the V3 locus (Moore, 1995); mABs 50.1 and 83.1 to the V3 loop (White-Scharf, 1993); MAbs IgG1b12 and F91 to the CD4 binding site (CD4bs) (Burton, 1994; and Moore, 1996) Mab 2G12 to a unique C3-V4 glycan-dependent epitope (Trkola, 1996) MAb M90 to the C1 region (diMarzo Veronese, 1992); Mab 23a and Ab D7324 to the C5 region (Moore, 1996); Mab 212A to a conformational C₁-C₅ epitope (Moore, 1994b); Mab 17b to a CD4-inducible epitope (Moore, 1996); Mab A32 to a CD4-inducible C₁-C₄ epitope (Moore, 1996; and Sullivan, 1998); Mabs G3-519 and G3-299 to C4 or C4/V3 epitopes (Moore, 1996). Mabs to gp41 epitopes included 7B2 to epitope cluster 1 (kindly provided by Jim Robinson, Tulane University); 25C2 to the fusion peptide region (Buchacher, 1994); 2F5 to a neutralizing epitope encompassing residues 665-690 (Muster, 1994). The tetrameric CD4-IgG2 has been described previously (Allaway, 1995).

Anti-HIV Antibodies were obtained from commercial sources, from the NIH AIDS Reagent Program, or from the inventor. Where indicated, the Antibodies were biotinylated with NHS-biotin (Pierce, Rockford, Ill.) according to the manufacturer's instructions.

Monomeric gp¹²⁰_{JR-FL} was produced in CHO cells stably transfected with the PPI4-tPA-gp120JR-FL plasmid as described (U.S. Pat. Nos. 5,866,163 and 5,869,624). Soluble CD4 was purchased from Bartels Corporation (Issaquah, Wash.).

Construction of PPI4-Based Plasmids Expressing Wild-Type and Mutant HIV Envelope Proteins

Wild-type gp140s (gp140WT). The gp140 coding sequences were amplified using the polymerase chain reaction (PCR) from full-length molecular clones of the HIV-1 isolates JR-FL, DH123, Gun-1, 89.6, NL4-3 and HxB2. The 5′ primer used was designated Kpnlenv (5′-GTCTATTATGGGGTACCTGTGTGGAA AGAAGC-3′) while the 3′ primer was BstBlenv (5′-CGCAGACGCAGATTCGAATT AATACCACAGCCAGTT-3′). PCR was performed under stringent conditions to limit the extent of Taq polymerase-introduced error. The PCR products were digested with the restriction enzymes Kpn1 and Xho1 and purified by agarose gel electrophoresis. Plasmid PPI4-tPA-gp120JR-FL was also digested with the two restriction enzymes and the large fragment (vector) was similarly gel-purified. The PPI4-tPA-gp120JR-FL expression vector has been described previously (U.S. Pat. Nos. 5,886,163 and 5,869,624). Ligations of insert and vector were carried out overnight at room temperature. DH5αF′Q10 bacteria were transformed with 1/20 of each ligation. Colonies were screened directly by PCR to determine if they were transformed with vector containing the insert. DNA from three positive clones of each construct was purified using a plasmid preparation kit (Qiagen, Valencia, Calif.) and both strands of the entire gp160 were sequenced. By way of example, pPPI4-gp140WTJR-FL and pPPI4-gp140WTDH123 refer to vectors expressing wild-type, cleavable gp140s derived from HIV-1_{JR-FL} and HIV-1_{DH123}, respectively.

gp140UNC. A gp120-gp41 cleavage site mutant of JR-FL gp140 was generated by substitutions within the REKR motif at the gp120 C-terminus, as described previously (Earl, 1990). The deletions were made by site-directed mutagenesis using the mutagenic primers 5′140M (5′-CTACGACTTCGTCTCCGCCTTCGACTACGG GGAATAGGAGCTGTGTTCCTTGGGTTCTTG-3′) and 3′gp140M (sequence conjunction with Kpnlenv and BstBlenv 5′-TCGAAGGCG GAGACGAAGTCGTAGCCGCAGTGCCTTGGTGGGTGCTACTCCTAATGGTTC-3′). In conjunction with Kpnlenv and BstB1, the PCR product was digested with Kpn1 and BstB1 and subcloned into pPPI4 as described above.

Loop-deleted gp120s and gp140s PPI4-based plasmids expressing variable loop-deleted forms of gp120 and gp140 proteins were prepared using the splicing by overlap extension method as described previously (Binley, 1998). In the singly loop-deleted mutants, a Gly-Ala-Gly spacer is used to replace D132-K152 (ΔV1), F156-I191 (ΔV2), or T300-G320 (ΔV3). The numbering system corresponds to that for the JR-FL clone of HIV-1 (Genbank Accession Number U63632).

PCR amplification using DGKPN5′PPI4 and 5JV1V2-B (5′-GTCTATTATGGGGTACCTGTGTGGAAAGAAGC-3′) on a ΔV1 template and subsequent digestion by Kpn1 and BamH1 generated a 292 bp fragment lacking the sequences encoding the V1 loop. This fragment was cloned into a plasmid lacking the sequences for the V2 loop using the Kpn1 and BamH1 restriction sites. The resulting plasmid was designated ΔV1V2′ and contained a Gly-Ala-Gly sequences in place of both D132-K152 and F156-I191. Envs lacking the V1, V2 and V3 loops were generated in a similar way using a fragment generated by PCR on a ΔV3 template with primers 3JV2-B (5′-GTCTGAGTCGGATCCTGTGA CACCTCAGTCATTACACAG-3′) and H₆NEW (5′CTCGAGTCTTCGAATTAGTGATG GGTGATGGTGATGATACCACAGCCATTTTGTTATGTC-3′). The fragment was cloned into ΔV1V2′, using BamH1 and BstB1. The resulting env construct was named ΔV1V2′V3. The glycoproteins encoded by the ΔV1V2′ and ΔV1V2′V3 plasmids encode a short sequence of amino acids spanning C125 to C130. These sequences were removed using mutagenic primers that replace T127-I191 with a Gly-Ala-Gly sequence. We performed PCR amplification with primers 3′DV1V2STU1 (5′-GGCTCAAAGGATATCTTTGGACAGGCCTGTGTAATG ACTGAGGTGTCACATCCTGCACCACAGAGTGGGGTTAATTTTACACATGGC-3′) and DGKPN5′PPI4, digested the resulting fragment by Stul and Kpn1 and cloned it in a PPI4 gp140 vector. The resulting gp140 was named ΔV1V2*. In an analogous manner ΔV1V2*V3 was constructed. The amino acid substitutions are shown schematically in FIG. 10.

Glycosylation site mutants. Canonical N-linked glycosylation sites were eliminated at positions 357 and 398 on gp120 by point mutations of asparagine to glutamine. These changes were made on templates encoding both wild-type and loop-deleted HIV envelope proteins.

Disulfide-stabilized gp140s. The indicated amino acids in gp120 and gp41 were mutated in pairs to cysteines by site-directed mutagenesis using the Quickchange™ kit (Stratagene, La Jolla, Calif.). As indicated below, additional amino acids in the vicinity of the introduced cysteines were mutated to alanines using similar methods in an attempt to better accommodate the cysteine mutations within the local topology of the envelope glycoproteins. The changes were similarly made on templates encoding both wild-type and loop-deleted HIV envelope proteins.

Expression of gp140s in transiently transfected 293T cells. HIV envelope proteins were transiently expressed in adherent 293T cells, a human embryonic kidney cell line (ATCC Cat. Number CRL-1573) transfected with the SV40 large T antigen, which promotes high level replication of plasmids such as PPI4 that contain the SV40 origin. 293T cells were grown in Dulbecco's minimum essential medium (DMEM; Life Technologies, Gaithersburg, Md.) containing 10% fetal bovine serum supplemented with L-glutamine, penicillin, and streptomycin. Cells were plated in a 10 cm dish and transfected with 10 μg of purified PPI4 plasmid using the calcium phosphate precipitation method. On the following day, cells were supplied fresh DMEM containing 0.2% bovine serum albumin along with L-glutamine, penicillin and streptomycin. For radioimmunoprecipitation assays, the medium also contained ³⁵S-labeled cysteine and methionine (200 μCi/plate). In certain experiments, the cells were cotransfected with 10 μg of a pcDNA3.1 expression vector (Invitrogen, Carlsbad, Calif.) encoding the gene for human furin.

ELISA analyses. The concentration of gp120 and gp140 proteins in 293T cell supernatants was measured by ELISA (Binley, 1997b). Briefly, Immulon II ELISA plates (Dynatech Laboratories, Inc.) were coated for 16-20 hours at 4° C. with a polyclonal sheep antibody that recognizes the carboxy-terminal sequence of gp120 (APTKAKRRVVQREKR). The plate was washed with tris buffered saline (TBS) and then blocked with 2% nonfat milk in TBS. Cell supernatants (100 μL) were added in a range of dilutions in tris buffered saline containing 10% fetal bovine serum. The plate was incubated for 1 hour at ambient temperature and washed with TBS. Anti-gp120 or anti-gp41 antibody was then added for an additional hour. The plate was washed with TBS, and the amount of bound antibody is detected using alkaline phosphatase conjugated goat anti-human IgG or goat anti-mouse IgG. Alternatively, biotinylated reporter Antibodies are used according to the same procedure and detected using a streptavidin-AP conjugate. In either case, AP activity is measured using the AMPAK kit (DAKO) according to the manufacturer's instructions. To examine the reactivity of denatured HIV envelope proteins, the cell supernatants were boiled for 5 minutes in the presence of 1% of the detergents sodium dodecyl sulfate and NP-40 prior to loading onto ELISA plates in a range of dilutions. Purified recombinant JR-FL gp120 was used as a reference standard.

Radioimmunoprecipitation assay (RIPA). ³⁵S-labeled 293T cell supernatants were collected 2 days post-transfection for RIPA analysis. Culture supernatants were cleared of debris by low speed centrifugation (˜300 g) before addition of RIPA buffer to a final concentration of 50 mM tris-HCl, 150 mM NaCl, 5 mM EDTA, pH 7.2. Biotinylated antibodies (˜10 μg) were added to 1 mL of supernatant and incubated at ambient temperature for 10 minutes. Samples were then incubated with streptavidin-agarose beads for 12-18 hours at 4° C. with gentle agitation. Alternatively, unlabeled antibodies were used in combination with protein G-agarose (Pierce, Rockford, Ill.). The beads were washed three times with RIPA buffer containing 1% Nonidet-P40 (NP40) detergent. Bound proteins were eluted by heating at 100° C. for 5 minutes with SDS-PAGE sample buffer containing 0.05M tris-HCl, 10% glycerol, 2% sodium dodecyl sulfate (SDS), 0.001% bromophenol blue, and where indicated, 100 mM dithiothreitol (DTT). Samples were loaded on an 8% polyacrylamide gel and run at 200V for 1 hour. Gels were then dried and exposed to a phosphor screen for subsequent image analysis using a STORM phosphoimager (Molecular Dynamics, Sunnyvale, Calif.). ¹⁴C-labeled proteins were used as size calibration standards (Life Technologies, Gaithersburg, Md.).

B. Results and Discussion

Processing of gp140NON is Facilitated by Co-Expression of the Furin Protease

To minimize the production of gp140NON, pcDNA3.1-furin and pPPI4-gp140WTJR-FL were cotransfected into 293T cells, and RIPA assay was performed using the anti-gp120 MAb 2G12. As indicated in FIG. 2, furin eliminated production of gp140NON but had no effect on gp140UNC. Similar results were obtained in RIPAs performed using other anti-gp120 MAbs (data not shown).

Treatment of the samples with DTT prior to SDS-PAGE did not affect the migration or relative amounts of these bands, indicating that the gp140s consist of a single polypeptide chain rather than separate gp120-gp41 molecules linked by an adventitious disulfide bond.

Stabilization of the gp120-gp41 Interaction by Introduction of Double Cysteine Mutations

With furin co-transfection, we could now express a soluble gp140 protein in which the gp120 and gp41ECTO components were associated only through a non-covalent linkage, mimicking what occurs in the native trimeric envelope glycoprotein complex on virions. However, on virions or the surface of infected cells, the gp120-gp41 association is weak, so that gp120 is gradually shed (McKeating, 1991). We found this to occur also with the gp140WT protein made in the presence of endogenous furin. Thus, we could detect very little, if any, stable gp120-gp41ECTO complexes in the supernatants from gp140WT-expressing cells after immunoprecipitation. We therefore sought ways to stabilize the non-covalent gp120-gp41 interaction, by the introduction of an intermolecular disulfide bond between the gp120 and gp41 subunits.

We therefore substituted a cysteine residue at one of several different positions in the C1 and C5 regions of gp120, focusing on amino acids previously shown to be important for the gp120-gp41 interaction (FIG. 3a).

Simultaneously, we introduced a second cysteine mutation at several residues near the intramolecular disulfide loop of gp41 (FIG. 3b). The intent was to identify pairs of cysteine residues whose physical juxtaposition in native gp120-gp41 was such that an intermolecular disulfide bond would form spontaneously. In all, >50 different double-cysteine substitution mutants were generated in the context of the JR-FL gp140WT protein, and co-expressed with furin in transient transfections of 293T cells.

An initial analysis of the transfection supernatants by antigen capture ELISA indicated that all of the mutants were efficiently expressed as secreted proteins, except those which contained a cysteine at residue 486 of gp120 (data not shown). We next characterized the transfection supernatants by immunoprecipitation with the anti-gp120 MAbs 2G12 and F91 (FIG. 4). In addition to the expected 120 kDa band (gp120), a second band of approximately 140 kDa was precipitated by F91 and 2G12 from many of the double-cysteine mutant transfection supernatants. The gp140 bands derived from mutants in which a cysteine was present in the C1 region of gp120 migrated slightly more slowly, and were more diffuse, than the corresponding bands from mutants in which the gp120 cysteine was in the CS region (FIG. 4). The presence of diffuse bands with reduced mobility on SDS-PAGE gels is probably indicative of incomplete or improper envelope glycoprotein processing, based on previous reports (Earl, 1990; and Earl, 1994). The relative intensity of the 140 kDa band was highly dependent upon the positions of the introduced cysteines, suggesting that certain steric requirements must be met if a stable intersubunit disulfide bond is to be formed.

To determine which among the double-cysteine mutants was the most suitable for further analysis, we determined the relative intensities of the gp140 and gp120 bands derived after immunoprecipitation of each mutant by the potently neutralizing anti-gp120 MAb 2G12, followed by SDS-PAGE and densitometry (FIG. 5). We sought the mutant for which the gp140/gp120 ratio was the highest, which we interpreted as indicative of the most efficient formation of the intermolecular disulfide bond. From FIG. 5, it is clear that mutant A492C/T596C has this property. From hereon, we will refer to this protein as the SOS gp140 mutant. Of note is that the mobility of the SOS gp140 mutant on SDS-PAGE is identical to that of the gp140NON protein, in which the gp120 and gp41ECTO moieties are linked by a peptide bond. The gp140 band derived from the SOS mutant is not quite as sharp as that from the gp140NON protein, but it is less diffuse than the gp140 bands obtained from any of the other double-cysteine mutants (FIG. 4). This suggests that the SOS mutant is efficiently processed. The complete nucleic acid and amino acid sequences of the JR-FL SOS gp140 mutant are provided in FIG. 13.

We verified that the 140 kDa proteins were stabilized by an intermolecular disulfide bond by treating the immunoprecipitated proteins with DTT prior to gel electrophoresis. In contrast, the 140 kDa bands in gp140WT and gp140UNC were unaffected by the DTT treatment as expected for uncleaved single-chain proteins. Of note is that a 140 kDa band was never observed for either the A492C or T596C single mutants (FIG. 6b). This is further evidence that the 140 kDa band in the double-cysteine mutants arises from the formation of an intermolecular disulfide bond between gp120 and gp41ECTO. In the absence of exogenous furin, the 140 kDa SOS protein band was not reducible by DTT, suggesting the band is the double cysteine mutant of gp140NON (FIG. 6c).

Approaches to Improve the Efficiency of Disulfide Bond Formation in the SOS gp140 Protein

Disulfide-stabilized gp140 is not the only env species present in the 293T cell supernatants. Discernable amounts of free gp120 are also present. This implies that the disulfide bond between gp120 and the gp41 ectodomain forms with imperfect efficiency. Although the free gp120 can be removed by the purification methods described below, attempts were made to further reduce or eliminate its production. To this end, additional amino acid substitutions were made near the inserted cysteines. In addition, the position of the cysteine in gp120 was varied. We retained the gp41 cysteine at residue 596, as in the SOS gp140 protein, because this position seemed to be the one at which intermolecular disulfide bond formation was most favored.

We first varied the position of the cysteine substitution in gp120, by placing it either N-terminal or C-terminal to alanine-492. The gp140/gp140+gp120 ratio was not increased in any of these new mutants; it remained comparable with, or less than, the ratio derived from the SOS gp140 protein (FIG. 7). Furthermore, there was usually a decrease in the mobility and sharpness of the gp140 band compared to that derived from the SOS gp140 protein (FIG. 7). Next, we considered whether the bulky side chains of the lysine residues adjacent to alanine-492 might interfere with disulfide bond formation. We therefore mutated the lysines at positions 491 and 493 to alanines in the context of the SOS gp140 protein, but these changes neither increased the gp140/gp140+gp120 ratio nor affected the migration of gp140 (FIG. 7). Finally, we introduced a second pair of cysteines into the SOS gp140 protein at residues 44 of gp120 and 600 of gp41, since a disulfide bond formed fairly efficiently when this cysteine pair was introduced into the wild-type protein (FIG. 5). However, the quadruple-cysteine mutant W44C/A492C/P600C/T596C was poorly expressed, implying that there was a processing or folding problem (FIG. 7). Poor expression was also observed with two more quadruple-cysteine mutants W44C/K491C/P600C/T596C and W44C/K493C/P600C/T596C (FIG. 7).

Further approaches to optimize the efficiency or overall expression of the disulfide stabilized mutant are possible. For example, cells stably transfected with furin could be created so as to ensure adequate levels of furin in all cells expressing the SOS gp140 proteins. Similarly, furin and the gp140 proteins could be coexpressed from a single plasmid. K491 and K493 could be mutated to non-alanine residues singly or as a pair. To better accommodate the introduced cysteines, other gp120 and/or gp41 amino acids in the vicinity of the introduced cysteines could be mutated as well.

The Antigenicity of the SOS gp140 Protein Parallels that of Virus-Associated gp120-gp41

Compared to gp140NON, the SOS gp140 protein has several antigenic differences that we believe are desirable for a protein intended to mimic the structure of the virion-associated gp120-gp41 complex. These are summarized below.

1) The SOS gp140 protein binds strongly to the potently neutralizing MAbs IgG1b12 and 2G12, and also to the CD4-IgG2 molecule (FIG. 8a). Although the RIPA methodology is not sufficiently quantitative to allow a precise determination of relative affinities, the reactivities of these MAbs and of the CD4-IgG2 molecule with the SOS gp140 protein appear to be substantially greater than with the gp140NON and gp120 proteins (FIG. 8a). Clearly, the SOS gp140 protein has an intact CD4-binding site. V3 loop epitopes are also accessible on the SOS gp140 protein, shown by its reactivity with MAbs 19b and 83.1 (FIG. 8a).

2) Conversely, several non-neutralizing anti-gp120 MAbs bind poorly, or not at all, to the SOS gp140 protein whereas they react strongly with gp140NON and gp120 (FIG. 8b). These MAbs include ones directed to the C1 and C5 domains, regions of gp120 that are involved in gp41 association and which are considered to be occluded in the context of a properly formed gp120-gp41 complex (Moore, 1994a; and Wyatt, 1997). Conversely, the C1- and C5-directed MAbs all reacted strongly with the gp140NON protein (FIG. 8b).

3) The exposure of the epitope for MAb 17b by the prior binding of soluble CD4 occurs far more efficiently on the SOS gp140 protein than on the gp140NoN or gp120 proteins (FIG. 8c). Indeed, in the absence of soluble CD4, there was very little reactivity of 17b with the SOS gp140 protein. The CD4-induced epitope for MAb 17b overlaps the coreceptor binding site on gp120; it is considered that this site becomes exposed on the virion-associated gp120-gp41 complex during the conformational changes which initiate virus-cell fusion after CD4 binding. Induction of the 17b epitope suggests that the gp120 moieties on the SOS gp140 protein possess the same static conformation and conformational freedom as virus-associated gp120-gp41. The gp140NON protein bound 17b constitutively, and although there was some induction of the 17b epitope upon soluble CD4 binding, this was less than occurred with the SOS gp140 protein.

4) Another CD4-inducible epitope on gp120 is that recognized by MAb A32 (Moore, 1996; and Sullivan, 1998). There was negligible binding of A32 to the SOS gp140 mutant in the absence of soluble CD4, but the epitope was strongly induced by soluble CD4 binding (FIG. 8c). As observed with 17b, the A32 epitope was less efficiently induced on the gp140NON protein than on the SOS gp140 protein.

5) There was no reactivity of any of a set of non-neutralizing gp41 MAbs with the SOS gp140 protein, whereas all of these MAbs bound strongly to the gp140NON protein. These anti-gp41 MAbs recognize several regions of the gp41 ectodomain, all of which are thought to be occluded by gp120 in the virion-associated gp120-gp41 complex (Moore, 1994a; and Sattentau, 1995). Their failure to bind to the SOS gp140 protein is another strong indication that this protein adopts a configuration similar to that of the native trimer; their strong recognition of the gp140NON protein is consistent with the view that these proteins have an aberrant conformation because of the peptide bond linking gp120 with gp41 (Edinger, 1999)(FIG. 8d).

6) In marked contrast to what was observed with the non-neutralizing MAbs, the neutralizing anti-gp41 MAb 2F5 bound efficiently to the SOS gp140 protein, but not to the gp140NON protein. Of note is that the 2F5 epitope is the only region of gp41 thought to be well exposed in the context of native gp120-gp41 complexes (Sattentau, 1995). Its ability to bind 2F5 is again consistent with the adoption by the SOS gp140 protein of a configuration similar to that of the native trimer.

The antigenic properties of the SOS gp140 protein were compared with those of the W44C/T596C gp140 mutant. Among the set of mutants that contained a cysteine substitution within the C1 domain, this was the most efficient at gp140 formation. Although the W44C/T596C gp140 reacted well with the 2G12 MAb, it bound CD4-IgG2 and IgG1b12 relatively poorly. Furthermore, there was little induction of the 17b epitope on the W44C/T596C gp140 by soluble CD4, yet strong reactivity with non-neutralizing anti-gp41 MAbs (FIG. 8). We therefore judge that this mutant has suboptimal antigenic properties. Indeed, the contrast between the properties of the W44C/T596C gp140 protein and the SOS gp140 protein demonstrates that the positioning of the intermolecular disulfide bonds has a significant influence on the antigenic structure of the resulting gp140 molecule.

In contrast to the antigenic character of the gp140SOS protein, the 140 kDa proteins of gp140WT and gp140UNC reacted strongly with non-neutralizing anti-gp120 and anti-gp41 MAbs such as G3-519 and 7B2. In addition, the epitope recognized by MAb 17B was constitutively exposed rather than CD4-inducible (FIG. 8e).

Overall, there was a strong correlation between the binding of MAbs to the SOS gp140 protein and their ability to neutralize HIV-1_{JR-FL}. This correlation was not observed with the gp140NON, gp140UNC or gp120 proteins.

The Formation of Intersubunit Disulfide Bonds is not Isolate-Dependent

To assess the generality of our observations with gp140 proteins derived from the HIV-1 isolate JR-FL, we generated double-cysteine mutants of gp140's from other HIV-1 strains. These include the R5X4 virus DH123 and the X4 virus HxB2. In each case, the cysteines were introduced at the residues equivalent to alanine-492 and threonine-596 of JR-FL. The resulting SOS proteins were transiently expressed in 293T cells and analyzed by RIPA to ascertain their assembly, processing and antigenicity. As indicated in FIG. 9, 140 kDa material is formed efficiently in the DH123 and HxB2 SOS proteins, demonstrating that our methods can successfully stabilize the envelope proteins of diverse viral isolates.

Disulfide Stabilization of HIV Envelope Proteins Modified in Variable Loop and Glycosylation Site Regions

Since there is evidence to suggest that certain variable loop and glycosylation site mutations provide a means to better expose underlying conserved neutralization epitopes, we examined the assembly and antigenicity of disulfide-stabilized forms. In initial studies, A492C/T596C JR-FL gp140 mutants were created for each of the ΔV1, ΔV2, ΔV3, ΔV1V1*, and ΔV1V2*V3 molecules described above. For the ΔV1V2*V3 protein, glycosylation site mutants were also synthesized by N-Q point mutations of amino acids 357 and 398.

For each of the singly and doubly loop-deleted mutants, we could detect gp140 bands in comparable quantities as for the full-length SOS gp140 protein (FIG. 11b). To see whether deletion of the variable loops altered antigenicity in an oligomeric context, we precipitated the ΔV3 and ΔV1V2* SOS proteins with a panel of MAbs (FIG. 12). MAbs to gp41 except 2F5 did not bind to loop deleted versions of the cysteine stabilized protein, indicating that those epitopes are still occluded. MAbs to C1 and C5 epitopes were similarly non-reactive. The neutralizing antibody 2F5 did bind to the mutants and was particularly reactive with the ΔV3 SOS protein. MAbs to the CD4BS (IgG1b12, F91) as well as 2G12 bound avidly to these mutants as well. Of note is that CD4-IgG2 and 2G12 bound with very high affinity to the oligomeric ΔV3 SOS protein. Furthermore, consistent with data indicating that the CD4i epitopes are constitutively exposed on the ΔV1V2* protein, binding of MAbs 17b and A32 to the ΔV1V2* SOS mutant was not inducible by sCD4. The ΔV3 SOS mutant, however, bound 17b and A32 weakly in the absence of sCD4 and strongly in its presence. These results are consistent with observations that the V1/V2 and V3 loop structures are involved in occlusion of the CD4i epitopes (Wyatt, 1995). Taken together, the results demonstrate that variable loop-deleted gp140s can be disulfide-stabilized without loss of conformational integrity. FIGS. 14 and 15, respectively, contain the complete nucleic acid and amino acid sequences of the ΔV1V2* and ΔV3 JR-FL SOS proteins.

For the ΔV1V2*V3 and ΔV1V2*V3 N357Q N398Q SOS mutants, we could not precipitate a gp140 (110 kDa and 105 kDa) with any of a variety of neutralizing and non-neutralizing MAbs (FIG. 11a, Lanes 3, 4, 7 & 8). We did, however, observe strong 90 kDa and 85 kDa bands, which correspond to the mutant gp120 domains. These preliminary experiments suggest a variety of approaches for disulfide-stabilizing triply-loop deleted gp140s, including adjusting the location(s) of one or more introduced cysteines, adding additional pairs of cysteines, modifying amino acids adjacent to the introduced cysteines, and modifying the manner in which the loops are deleted. Alternatively, triply loop deleted gp140s derived from other HIV isolates may be more readily stabilized by cysteines introduced at residues homologous to 496/592.

Production and Purification of Recombinant HIV-1 Envelope Glycoproteins

Milligram quantities of high quality HIV-1 envelope glycoproteins are produced in CHO cells stably transfected with PPI4 envelope-expressing plasmids (U.S. Pat. Nos. 5,886,163 and 5,869,624). The PPI4 expression vector contains the dhfr gene under the control of the β-globin promoter. Selection in nucleoside-free media of dhfr+ clones is followed by gene amplification using stepwise increases in methotrexate concentrations. The cytomegalovirus (CMV) promoter drives high level expression of the heterologous gene, and the tissue plasminogen activator signal sequence ensures efficient protein secretion. A high level of gp120 expression and secretion is obtained only upon inclusion of the complete 5′ non-coding sequences of the CMV MIE gene up to and including the initiating ATG codon. To produce milligram quantities of protein, recombinant CHO cells are seeded into roller bottles in selective media and grown to confluency. Reduced serum-containing media is then used for the production phase, when supernatants are harvested twice weekly. A purification process comprising lectin affinity, ion exchange, and/or gel filtration chromatography is carried out under non-denaturing conditions.

A Protocol for Determining the Immunogenicity of Stabilized HIV-1 Envelope Subunit Proteins

Purified recombinant HIV-1 envelope proteins are formulated in suitable adjuvants (e.g., Alum or Ribi Detox). For alum, formulation is achieved by combining the mutant HIV-1 envelope glycoprotein (in phosphate buffered saline, normal saline or similar vehicle) with preformed aluminum hydroxide gel (Pierce, Rockford, Ill.) at a final concentration of approximately 500 μg/mL aluminum. The antigen is allowed to adsorb onto the alum gel for two hours at room temperature. Guinea pigs or other animals are immunized 5 times, at monthly intervals, with approximately 100 μg of formulated antigen, by subcutaneous intramuscular or intraperitoneal routes. Sera from immunized animals are collected at biweekly intervals and tested for reactivity with HIV-1 envelope proteins in ELISA as described above and for neutralizing activity in well established HIV-1 infectivity assays (Trkola, 1998). Vaccine candidates that elicit the highest levels of HIV-1 neutralizing Antibodies can be tested for immunogenicity and efficacy in preventing or treating infection in SHIV-macaque or other non-human primate models of HIV infection, as described below. The subunit vaccines could be used alone or in combination with other vaccine components, such as those designed to elicit a protective cellular immune response.

For these studies, the HIV-1 envelope proteins also may be administered in complex with one or more cellular HIV receptors, such as CD4, CCR5, and CXCR4. As described above, the binding of soluble CD4 exposes formerly cryptic conserved neutralization epitopes on the stabilized HIV-1 envelope protein. Antibodies raised to these or other neoepitopes could possess significant antiviral activity. As described above, interaction of CD4-env complexes with fusion coreceptors such as CCR5 and CXCR4 is thought to trigger additional conformational changes in env required for HIV fusion. Trivalent complexes comprising the stabilized env, CD4, and coreceptor could thus adopt additional fusion intermediary conformations, some of which are thought to be sufficiently long-lived for therapeutic and possibly immunologic interventions (Kilby, 1998). Methods for preparing and administering env-CD4 and env-CD4-coreceptor complexes are well-known to the skilled artisan (LaCasse, 1999; Kang, 1994; and Gershoni, 1993).

A Protocol for Determining the Immunogenicity of Nucleic Acid-Based Vaccines Encoding Stabilized HIV-1 Envelope Proteins

PCR techniques are used to subclone the nucleic acid into a DNA vaccine plasmid vector such as pVAX1 available from Invitrogen (catalog number V260-20). PVAX1 was developed according to specifications in the FDA document “Points to Consider on Plasmid DNA Vaccines for Preventive Infectious Disease Indications” published on Dec. 22, 1996. PVAX1 has the following features: Eukaryotic DNA sequences are limited to those required for expression in order to minimize the possibility of chromosomal integration, Kanamycin is used to select the vector in E. coli because ampicillin has been reported to cause an allergic response in some individuals, Expression levels of recombinant proteins from pVAX1 is comparable to those achieved with its parent vector, pc DNA3.1, and the small size of pVAX1 and the variety of unique cloning sites amplify subcloning of even very large DNA fragments.

Several methods can be used to optimize expression of the disulfide stabilized protein in vivo. For example, standard PCR cloning techniques could be used to insert into pVAX1 certain elements of the optimized PPI4 expression vector, including Intron A and adjoining regions of the CMV promoter. In addition, the genomic DNA sequences of the HIV-1 envelope are biased towards codons that are suboptimal for expression in mammalian cells (Haas, 1996). These can be changed to more favorable codons using standard mutagenesis techniques in order to improve the immunogenicity of nucleic acid based HIV vaccines (Andre, 1998). The codon optimization strategy could strive to increase the number of CpG motifs, which are known to increase the immunogenicity of DNA vaccines (Klinman, 1997). Lastly, as for the transient transfection systems described above, env processing into gp120-gp41 may be facilitated by the heterologous expression of furin introduced on the same or separate expression vectors.

The insert containing plasmid can be administered to the animals by such means as direct injection or using gene gun techniques. Such methods are known to those skilled in the art.

In one protocol, Rhesus macaques are individually inoculated with five approximately 1 mg doses of the nucleic acid. The doses are delivered at four week intervals. Each dose is administered intramuscularly. The doses are delivered at four week intervals. After four months, the animals receive a single immunization at two separate sites with 2 mg of nucleic acid with or without 300 μg of mutant HIV-1 envelope glycoprotein. This series may be followed by one or more subsequent recombinant protein subunit booster immunizations. The animals are bled at intervals of two to four weeks. Serum samples are prepared from each bleed to assay for the development of specific antibodies as described in the subsequent sections.

SHIV Challenge Experiments

Several chimeric HIV-SIV viruses have been created and characterized for infectivity in Rhesus monkeys. For Virus challenge experiments, the Rhesus monkeys are injected intravenously with a pre-titered dose of virus sufficient to infect greater than 9/10 animals. SHIV infection is determined by two assays. ELISA detection of SIV p27 antigen in monkey sera is determined using a commercially available kit (Coulter). Similarly, Western blot detection of anti-gag antibodies is performed using a commercially available kit (Cambridge Biotech).

A reduction in either the rate of infection or the amount of p27 antigen produced in immunized versus control monkeys would indicate that the vaccine or vaccine combination has prophylactic value.

### Experimental Set II

A. Synopsis of Results

The gp120 and gp41 subunits of the human immunodeficiency virus type 1 (HIV-1) envelope glycoprotein associate via weak, non-covalent interactions, which can be stabilized by an intersubunit disulfide bond between cysteine residues introduced at appropriate sites in gp120 and gp41. The properties of such a protein, designated SOS gp140, are described herein. HIV-1_{JR-FL} SOS gp140, proteolytically uncleaved gp140 (gp140UNC) and gp120 were expressed in stably transfected Chinese hamster ovary (CHO) cells and analyzed for antigenic and structural properties before and after purification. In surface plasmon resonance (SPR) and radioimmunoprecipitation assays, SOS gp140 avidly bound the broadly neutralizing monoclonal antibodies (MAbs) 2G12 (anti-gp120) and 2F5 (anti-gp41), whereas gp140UNC bound these MAbs less avidly. In addition, MAb 17b against a CD4-induced epitope that overlaps the CCR5-binding site bound more strongly and rapidly to SOS gp140 than to gp140UNC. In contrast, gp140UNC displayed the greater reactivity with non-neutralizing anti-gp120 and anti-gp41 MAbs. A series of immunoelectron microscopy studies suggested a model for SOS gp140 wherein the gp41 ectodomain (gp41ECTO) occludes the “non-neutralizing” face of gp120, consistent with the antigenic properties of this protein. Also discussed is the application of Blue Native polyacrylamide gel electrophoresis (BN-PAGE), a high-resolution molecular sizing method, to the study of viral envelope proteins in purified and unpurified form. BN-PAGE and other biophysical studies demonstrated that SOS gp140 was monomeric, whereas gp140UNC comprised a mixture of non-covalently associated and disulfide-linked oligomers that could be resolved into dimers, trimers and tetramers by BN-PAGE. The oligomeric and antigenic properties of these proteins were largely unaffected by purification. An uncleaved gp140 protein containing the SOS cysteine mutations (SOS gp140UNC) was also oligomeric, indicating that cleavage of an oligomeric gp140 protein into gp120 and gp41 subunits destabilizes the gp41-gp41 interactions. This may be necessary for fusion to occur, but hinders the production of recombinant envelope glycoprotein complexes that mimic the native, virion-associated structure. Surprisingly, variable-loop-deleted SOS gp140 proteins were expressed as cleaved, non-covalently associated oligomers that were significantly more stable than the full-length protein. This suggests one path for producing proteolytically mature forms of the HIV-1 envelope glycoproteins in purified, oligomeric form. Overall, our findings have relevance for rational vaccine design.

B. Introduction

HIV vaccine development targeting HIV envelope glycoproteins has been hindered by the inherent instability of the native envelope glycoprotein complex. Therefore, more stable forms of the envelope glycoprotein complex that better mimic the native structure need to be developed.

An approach to resolving the instability of the native complex is to remove the cleavage site that naturally exists between the gp120 and gp41 subunits. Doing so means that proteolysis of this site does not occur, leading to the expression of gp140 glycoproteins in which the gp120 subunit is covalently linked to the gp41 ectodomain (gp41ECTO) by means of a peptide bond (Berman, 1990; Berman, 1988; Earl, 1997; Earl, 1994; and Earl, 1990). Such proteins can be oligomeric, sometimes trimeric (Chen, 2000; Earl, 1997; Earl, 1994; Earl, 1990; Earl, 2001; Edinger, 2000; Farzan, 1998; Richardson, 1996; Stamatatos, 2000; Yang, 2000a; Yang, 2000b; Yang, 2001; and Zhang, 2001).

However, it is not clear that they truly represent the structure of the native, fusion-competent complex in which the gp120-gp41 cleavage site is fully utilized. Hence the receptor-binding properties of uncleaved gp140 (gp140UNC) proteins tend to be impaired, and non-neutralizing antibody epitopes are exposed on them that probably are not accessible on the native structure (Binley, 2000a; Burton, 1997; Hoffman, 2000; Sattentau, 1995; and Zhang, 2001).

An alternative approach to the problem of gp120-gp41 instability, is to retain the cleavage site but to introduce a disulfide bond between the gp120 and gp41ECTO subunits (Binley, 2000a; and Sanders, 2000). Properly positioned, this intermolecular disulfide bond forms efficiently during envelope glycoprotein (Env) synthesis, allowing the secretion of gp140 proteins that are proteolytically processed but in which the association between the gp120 and gp41ECTO subunits is maintained by the disulfide bond.

Here we show that the gp41-gp41 interactions are unstable in the SOS gp140 protein, which is expressed and purified primarily as a monomer. In contrast, gp140UNC proteins, with or without the SOS cysteine substitutions, are multimeric, implying that cleavage of the peptide bond between gp120 and gp41 destabilizes the native complex. Despite being monomeric, the purified and unpurified forms of SOS gp140 are better antigenic structural mimics of the native, fusion-competent Env structure than are the corresponding gp120 or gp140UNC proteins. This may be because the presence and orientation of gp41ECTO occludes certain non-neutralization epitopes on SOS gp140 while preserving the presentation of important neutralization sites. This explanation is consistent with immunoelectron microscopy studies of the protein. Unexpectedly, proteolytically mature, but variable-loop-deleted, SOS gp140 glycoproteins have enhanced oligomeric stability, so these molecules warrant further study for their structural and immunogenic properties.

C. Materials and Methods

Plasmids. The pPPI4 eukaryotic expression vectors encoding SOS and uncleaved forms of HIV-1_{JR-FL} gp140 have been described previously (Binley, 2000a; and Trkola, 1996). The SOS gp140 protein contains cysteine substitutions at residues A501 in the C5 region of gp120 and T605 in gp41 (Binley, 2000a; and Sanders, 2000). In gp140UNC, the sequence KRRVVQREKRAV at the junction between gp120 and gp41ECTO has been replaced with a hexameric LR motif to prevent scission of gp140 into gp120 and gp41ECTO (Binley, 2000a). Plasmids encoding variable-loop-deleted forms of HIV-1_{JR-FL} SOS gp140 have been described (Sanders, 2000). In these constructs, the tripeptide GAG is used to replace V1 loop sequences (D133-K155) and V2 loop sequences (F159-1194), alone or in combination. The SOS gp140UNC protein contains the same cysteine substitutions that are present in SOS gp140, but the residues REKR at the gp120-gp41ECTO cleavage site have been replaced by the sequence IEGR, to prevent gp140 cleavage. The furin gene (Thomas, 1988) was expressed from plasmid pcDNA3.1furin (Binley, 2000a).

MAbs and CD4-based proteins. The following anti-gp120 MAbs were used: IgG1b12 [against the CD4 binding site (Burton, 1994)], 2G12 [against a unique C3-V4 glycan-dependent epitope (Trkola, 1996)], 17b [against a CD4-inducible epitope (Thali, 1993), 19b [against the V3 loop (Moore, 1995)], and 23A [against the C5 region (Moore, 1996)]. The anti-gp41 MAbs were 2F5 [against a cluster 1 epitope centered on the sequence ELDKWA (Muster, 1993; and Parker, 2001)] and 2.2B [against epitope cluster II]. MAbs IgG1b12, 2G12 and 2F5 are broadly neutralizing (Trkola, 1995). MAb 17b weakly neutralizes diverse strains of HIV-1, more so in the presence of soluble CD4 (Thali, 1993), whereas the neutralizing activity of MAb 19b against primary isolates is limited (Trkola, 199-8). MAbs 23A and 2.2B are non-neutralizing. Soluble CD4 (sCD4) and the CD4-based molecule CD4-IgG2 have been described elsewhere (Allaway, 1995).

HIV-1 gp140 and gp120 glycoproteins. To create stable cell lines that secrete full-length HIV-1_{JR-FL} SOS gp140 or ΔV1V2 SOS gp140, we co-transfected DXB-11 dihydrofolate reductase (dhfr)-negative CHO cells with pcDNA3.1furin and either pPPI4—SOS gp140 (Binley, 2000a) or pPPI4-ΔV1V2* SOS gp140 (Sanders, 2000), respectively, using the calcium phosphate precipitation method. Doubly transformed cells were selected by passaging the cells in nucleoside-free α-MEM media containing 10% fetal bovine serum (FBS), geneticin (Life Technologies, Rockville, Md.) and methotrexate (Sigma, St.

Louis, Mo.). The cells were amplified for gp140 expression by stepwise increases in methotrexate concentration, as described elsewhere (Allaway, 1995). Clones were selected for SOS gp140 expression, assembly, and endoproteolytic processing based on SDS-PAGE and Western blot analyses of culture supernatants. CHO cells expressing SOS gp140UNC were created using similar methods, except that pcDNA3.1furin and geneticin were not used. Full-length SOS gp140 was purified from CHO cell culture supernatants by Galanthus nivalis lectin affinity chromatography (Sigma) and Superdex 200 gel filtration chromatography (Amersham-Pharmacia, Piscataway, N.J.), as described elsewhere (Trkola, 1996). The gp140UNC glycoprotein was purified by lectin chromatography only. The concentration of purified Envs was measured by UV spectroscopy as described (Scandella, 1993), and was corroborated by ELISA and densitometric analysis of SDS-PAGE gels. Recombinant HIV-1_{JR-FL}, HIV-1_{LAI} and HIV-1_{YU2} gp120 glycoproteins were produced using methods that have been previously described (Trkola, 1996; and Wu, 1996).

Where indicated, HIV-1 envelope glycoproteins were transiently expressed in adherent 293T cells by transfection with Env- and furin-expressing plasmids, as described previously (Binley, 2000a). For radioimmunoprecipitation assays, the proteins were metabolically labeled with [³⁵S]cysteine and [³⁵S]methionine for 24 hour prior to analysis.

SDS-PAGE, radioimmunoprecipitation, Blue Native PAGE, and Western blot analyses. Sodium dodecyl sulfate polyacrylamide gel electrophoresis (SDS-PAGE) analyses were performed as described elsewhere (Binley, 2000a). Reduced and non-reduced samples were prepared by boiling for 2 minutes in Laemnli sample buffer (62.5 mM Tris-HCl, pH 6.8, 2% SDS, 25% glycerol, 0.01% bromophenol blue) in the presence or absence, respectively, of 50 mM dithiothreitol (DTT). Protein purity was determined by densitometric analysis of the stained gels followed by the use of ImageQuant software (Molecular Devices, Sunnyvale, Calif.). Radioimmunoprecipitation assays (RIPA) were performed on Env-containing cell culture supernatants, as previously described (Binley, 2000a; and Sanders, 2000).

Blue Native (BN)—PAGE was carried out with minor modifications to the published method (Schagger, 1994; and Schagger, 1991). Thus, purified protein samples or cell culture supernatants were diluted with an equal volume of a buffer containing 100 mM 4-(N-morpholino)propane sulfonic acid (MOPS), 100 mM Tris-HCl, pH 7.7, 40% glycerol, 0.1% coomassie blue, just prior to loading onto a 4-12% Bis-Tris NuPAGE gel (Invitrogen). Typically, gel electrophoresis was performed for 2 h at 150V (˜0.07A) using 50 mM MOPS, 50 mM Tris, pH 7.7, 0.002% coomassie blue as cathode buffer, and 50 mM MOPS, 50 mM Tris, pH 7.7 as anode buffer. When purified proteins were analyzed, the gel was destained with several changes of 50 mM MOPS, 50 mM Tris, pH 7.7 subsequent to the electrophoresis step. Typically, 5 μg of purified protein were loaded per lane.

For Western blot analyses, gels and polyvinylidine difluoride (PVDF) membranes were soaked for 10 minutes in transfer buffer (192 mM glycine, 25 mM Tris, 0.05% SDS, pH 8.8 containing 20% methanol). Following transfer, PVDF membranes were destained of coomassie blue dye using 25% methanol and 10% acetic acid and air-dried. Destained membranes were probed using the anti-V3 loop MAb PA1 (Progenics) followed by horseradish peroxidase (HRP)-labeled anti-mouse IgG (Kirkegaard & Perry Laboratories, Gaithersburg, Md.), each used at 0.2 μg/mL final concentration. Luminometric detection of the envelope glycoproteins was obtained with the Renaissance7 Western Blot Chemiluminescence Reagent Plus system (Perkin Elmer Life Sciences, Boston, Mass.). Bovine serum albumin (BSA), apo-ferritin, and thyroglobulin were obtained from Amersham Biosciences (Piscataway, N.J.) and used as molecular weight standards.

Matrix-assisted laser desorption/ionization time-of-flight (MALDI-TOF) mass spectrometry. Proteins were dialyzed overnight against water prior to analysis. Where indicated, SOS gp140 (1 mg/ml) was reduced with 10 mM DTT (Sigma), after which iodoacetamide (Sigma) was added to a final concentration of 100 mM, before dialysis. The samples were mixed with an equal volume of sinapinic acid matrix solution, dried at room temperature, and analyzed by MALDI-TOF mass spectrometry (Lewis, 1998). MALDI-TOF mass spectra were acquired on a PerSeptive Biosystems Voyager-STR mass spectrometer with delayed extraction. Samples were irradiated with a nitrogen laser (Laser Science Inc.) operated at 337 nm. Ions produced in the sample target were accelerated with a deflection voltage of 30,000V.

Sedimentation equilibrium analysis. Sedimentation equilibrium measurements were performed on a Beckman XL-A Optima analytical ultracentrifuge with an An-60 Ti rotor at 20° C. Protein samples were dialyzed overnight against 50 mM sodium phosphate. (pH 7.0) and 150 mM NaCl, loaded at initial concentrations of 0.25 mM, 0.5 mM and 1 mM, then centrifuged in a six-sector cell at rotor speeds of 6,000 and 9,000 rpm. Data were acquired at two wavelengths per rotor speed and processed simultaneously with a nonlinear least squares fitting routine (Johnson, 1981). Solvent density and protein partial specific volume were calculated according to solvent and protein composition, respectively (Laue, 1992).

Size exclusion chromatography. Purified, CHO cell-expressed SOS gp140, gp140UNC and gp120 proteins were analyzed by size exclusion chromatography on a TSK G3000SWXL HPLC column (TosoHaas, Montgomeryville, Pa.) using phosphate buffered saline (PBS) as the running buffer. The protein retention time was determined by monitoring the UV absorbance of the column effluent at a wavelength of 280 nm. The column was calibrated using ferritin as a model protein that exists in oligomeric states of 220 kDa, 440 kDa and 880 kDa (Gerl, 1988).

Surface Plasmon Resonance Measurements

Immunoelectron microscopy. Immunoelectron-microscopic analyses of SOS gp140 and gp120 alone and in complex with MAb, MAb fragments and sCD4 were performed by negative staining with uranyl formate as previously described (Roux, 1989; and Roux, 1996). The samples were examined on a JEOL JEM CX-100 electron microscope and photographed at 100,000 diameters magnification.

Immune complex image digitalizing and averaging. The electron micrographs of immune complex images were digitalized on an AGFA DUOSCAN T2500 Negative Scanner (Ridgefield Park, N.J.). Potentially informative complexes were selected and windowed as 256H 256-pixel images. These randomly oriented complexes were then brought into approximate alignment utilizing the multi-reference alignment function of the SPIDER program (Frank, 1996). The aligned images were subsequently averaged to improve the signal-to-noise ratio.

Molecular modeling. The SwissPDBviewer program (Guex, 1997) was used to enhance the EM-based interpretations and to investigate the likely location of the gp41 domain in SOS gp140.

D. Results

Assembly and Cleavage of Purified SOS gp140

We have previously described the antigenic properties of unpurified HIV-1_{JR-FL} SOS gp140 proteins produced via transient transfection of 293T cells (Binley, 2000a). To facilitate preparation of larger amounts of this protein for evaluation in purified form, we constructed a stable CHO cell line that expresses both SOS gp140 and human furin. Heterologous furin was expressed to facilitate efficient proteolytic processing of SOS gp140 (Binley, 1997b).

The SOS gp140 protein was purified from CHO cell supernatants to ˜90% homogeneity (FIG. 16, Lane 8). Only minor amounts of free gp120 were present in the SOS gp140 preparation, indicating that the inter-subunit disulfide bond remained substantially intact during purification. No high molecular weight SOS gp140 oligomers or aggregates were observed (FIG. 16, Lane 8). Under non-reducing conditions, SOS gp140 migrated as a predominant 140 kDa band. The major contaminant was bovine alpha 2-macroglobulin, which migrates as an ˜170 kDa band on a reducing SDS-PAGE gel (FIG. 16, Lane 3) and can be eliminated by adaptation of the CHO cell line to serum-free culture (data not shown). Upon reduction with DTT, the purified SOS gp140 protein migrated as a predominant 120 kDa band, with a minor (˜10%) fraction of the 140 kDa band present (FIG. 16, Lane 3). These data indicated that approximately 90% of the SOS gp140 protein was proteolytically processed.

The HIV-1_{JR-FL} gp140UNC protein was expressed in CHO cells using similar methods, although without co-transfected furin, and was also obtained at ˜90% purity. It too contained alpha 2-macroglobulin as the major contaminant, but no free gp120 was detectable (FIG. 16, Lanes 4 and 9). In the absence of DTT, alpha 2-macroglobulin migrates as a ˜350 kDa dimer and is not clearly resolved from gp140UNC oligomers (FIG. 16, Lane 9). Under non-reducing conditions, bands consistent with gp140UNC monomers (140 kDa), dimers (280 kDa), and trimers (420 kDa) were observed in roughly equal amounts (FIG. 16, Lane 9). These proteins were reactive with anti-gp120 MAbs in Western blot analysis (data not shown). When treated with DTT, gp140UNC gave rise to an intensified monomer band at 140 kDa and an alpha 2-macroglobulin monomer band at ˜170 kDa; but gp140 oligomers were absent (FIG. 16, compare Lanes 4 and 9). Thus, disulfide-linked, reducible oligomers comprise half or more of the gp140UNC preparation. Comparable amounts of reducible oligomers have been observed in gp140UNC protein preparations derived from subtype A, B and E viruses, with minor strain-to-strain differences (Owens, 1999; and Staropoli, 2000). Reducible gp160 oligomers of this type have been proposed to contain aberrant intermolecular disulfide bonds (Owens, 1999). If so, at least some of the oligomers present in gp140UNC preparations represent misfolded protein aggregates.

Biophysical Properties of Purified SOS gp140

Matrix-assisted laser desorption ionization mass spectrometry. This technique was used to determine the absolute molecular masses of HIV-1_{JR-FL} gp120 and SOS gp140. As indicated in Table 1 (shown below), the measured molecular masses were 121.9 kDa for SOS gp140 and 91.3 kDa for gp120.

TABLE 1
Molecular masses of recombinant HIV-1_{JR-FL} envelope
glycoproteins as determined by MALDI-TOF mass spectrometry

| HIV-1_{JR-FL} envelope glycoprotein | mass, kDa |
|---|---|
| gp120 | 91.3 |
| SOS gp140 | 121.9 |
| SOS gp140, reduced: | |
| uncleaved gp140 | 118.5 |
| gp120 | 91.8 |
| gp41ECTO | 27.0 |

Reduced SOS gp140 gave rise to a small peak of uncleaved gp140 at 118.5 kDa, a gp120 peak at 91.8 kDa and a gp41ECTO peak at 27 kDa. Differences in glycosylation between cleaved and uncleaved SOS gp140 proteins could account for the 3.4 kDa difference in their measured masses. A smaller difference (˜500 Da) was observed in the mass of gp120 when it was expressed alone and in the context of SOS gp140. The alanine 6 cysteine SOS mutation would be expected to increase the mass of gp120 by only 32 Da (one sulfur atom), so again a minor difference in glycosylation patterns may be responsible. The measured mass of HIV-1_{JR-FL} gp120 is comparable to previously reported molecular masses of CHO cell-expressed HIV-1_{GBB} gp120 (91.8 kDa) and Drosophila cell-expressed HIV-1_{WD61} gp120 (99.6 kDa) (Jones, 1995; and Myszka, 2000). The anomalously high molecular weights (˜120 kDa and ˜140 kDa, respectively, FIG. 16) observed for gp120 and SOS gp140 by SDS-PAGE reflect the high carbohydrate content of these proteins. The extended structure of the glycans and their poor reactivity with the dodecyl sulfate anion retard the electrophoretic migration of the glycoproteins through SDS-PAGE gel matrices (Jones, 1995).

Ultracentrifugation sedimentation equilibrium measurements were used to examine the oligomeric state of purified SOS gp140. Over protein concentrations ranging from 0.25-1.0 mM, the apparent molecular weight of SOS gp140 was consistently found to be 155 kDa (FIG. 17a). Hence, the purified SOS gp140 protein is monomeric in solution. There was no systematic dependence of molecular weight on protein concentration over the range studied. However, the residuals (the difference between the data and the theoretical curve for a monomer) deviated from zero in a systematic fashion (FIG. 17a), suggesting the presence of small amounts of oligomeric material.

Analytical gel filtration chromatography Purified HIV-1_{JR-FL} SOS gp140, gp140UNC and gp120 proteins were also examined using size exclusion chromatography. Monomeric gp120 eluted with a retention time of 6.24 minutes and an apparent molecular weight of ˜200 kDa (FIG. 17b). The apparently large size of this protein reflects the extended structures of its carbohydrate moieties. The retention time (5.95 minutes) and apparent molecular weight (˜220 kDa) of the SOS gp140 protein are consistent with it being a monomer that is slightly larger than gp120. In contrast, the gp140UNC protein eluted at 4.91 minutes as a broad peak with an average molecular weight of >500 kDa, which is consistent with it comprising a mixture of oligomeric species. Although the chromatogram suggests the existence of multiple species in the gp140UNC preparation, this gel-filtration technique cannot resolve mixtures of gp140 dimers, trimers and tetramers.

Blue Native polyacrylamide gel electrophoresis BN-PAGE was used to examine the oligomeric state of the purified SOS gp140 and gp140UNC proteins. In BN-PAGE, most proteins are fractionated according to their Stokes' radius. We first applied this technique to a model set of soluble proteins, including gp120 alone and in complex with sCD4 (FIG. 17c). The model proteins included thyroglobulin and ferritin, which naturally comprise a distribution of non-covalent oligomers of varying size. The oligomeric states of these multi-subunit proteins, as determined by BN-PAGE, are similar to those observed using other non-denaturing techniques (Gerl, 1988; and Venkatesh, 1999). BSA exists as monomers, dimers, and higher order species in solution (Lambin, 0.1982); the same ladder of oligomers was observed in BN-PAGE. Not surprisingly, the gp120/sCD4 complex, which has an association constant in the nanomolar range (Allaway, 1995), remained intact during BN-PAGE analysis.

The purified SOS gp140 protein was largely monomeric by BN-PAGE (FIG. 17d), although a minor amount (<10%) of dimeric species was also observed. The purified gp140UNC protein migrated as well-resolved dimers, trimers and tetramers, with trace amounts of monomer present (FIG. 17d). The gp140UNC dimer represented the major oligomeric form of the protein present under non-denaturing conditions. Although tetrameric gp140UNC is a distinct minor species on BN-PAGE gels (FIG. 17d), it is absent from non-reduced SDS-PAGE gels (FIG. 16). Upon treatment with SDS and heat, the gp140UNC tetramers probably revert to lower molecular weight species, such as monomers and/or disulfide-linked dimers. As expected, HIV-1_{JR-FL} gp120 migrated as a predominant 120 kDa monomeric protein. BN-PAGE analyses of unpurified gp140 proteins are described below (see FIG. 23).

Overall, ultracentrifugation, gel filtration and BN-PAGE analyses were in excellent agreement as to the oligomeric states of these purified Env proteins. BN-PAGE, however, was the only method capable of clearly resolving the mixture of oligomeric species contained in the gp140UNC preparation.

Immunoelectron Microscopy of SOS gp140 and SOS gp140-MAb Complexes

In the absence of antibodies, the electron micrographs revealed SOS gp140 to be mostly monomeric, randomly oriented and multi-lobed (FIG. 18a). Qualitatively similar images were obtained with HIV-1_{JR-FL} gp120 (data not shown), and the two proteins could not be clearly distinguished in the absence of MAbs or other means of orienting the images.

Electron micrographs were also obtained of SOS gp140 in complex with MAbs 2F5 (FIG. 18b), IgG1b12 (FIG. 18c) and 2G12 (FIG. 18d). To aid in interpretation, the complexes were masked and rotated such that the presumptive Fc of the MAb points downward. Schematic diagrams are also provided for each complex in order to illustrate the basic geometry and stoichiometry observed. In each case, the complexes shown represent the majority or plurality species present. However, other species, such as free MAb and monovalent MAb-SOS gp140 complexes, were also present in each sample (data not shown).

When combined with IgG1b12 or 2F5, SOS gp140 formed rather typical immune complexes composed of a single MAb and up to two SOS gp140s (FIGS. 18b and 18c). The complexes adopted the characteristic Y-shaped antibody structure, with a variable angle between the Fab arms of the MAb. In contrast, the 2G12/SOS gp140 complexes produced strikingly different images (FIG. 18d). Y-shaped complexes comprising two distinct Fab arms with bound SOS gp140s were rare. Instead the 2G12-SOS gp140 images were strongly linear and appeared to represent one MAb bound to two SOS gp140 proteins aligned in parallel. The parallel alignment of the SOS gp140s forces the two Fab arms into similar alignment, resulting in an overall linear structure. These complexes are unprecedented in our immunoelectron microscopy studies of Env-MAb complexes (Roux, 1989; Roux, 1996; and Zhu, 2001) and KHR, unpublished observations). Of note is that the HIV-1_{JR-FL} gp120-2G12 complexes do not adopt this parallel configuration but instead resemble the SOS gp140-2F5 and SOS gp140-IgG1b12 complexes (data not shown). One hypothesis is that 2G12 binds to SOS gp140 in an orientation that promotes residual weak interactions between the gp41ECTO moieties, which then stabilize the complex in the parallel configuration observed. Additional studies are ongoing to further explore this finding.

Combinations of the above, well-characterized MAbs were used to examine the relative placement of their epitopes on SOS gp140. In the first combination, SOS gp140-2F5-IgG1b12, multiple ring structures were observed which appeared to be composed of two SOS gp140 proteins bridged by two antibody molecules (data not shown). To distinguish between the 2F5 and IgG1b12 MAbs, we examined complexes formed between IgG1b12 F(ab′)2, SOS gp140 and the intact 2F5 MAb. Characteristic ring structures were again observed (FIG. 18e). The ring complexes were then subjected to computational analysis using the SPIDER program package to yield several categories of averaged images (data not shown). The MAb 2F5 and IgG1b12 F(ab′)2 components can clearly be delineated in the images, as can the SOS gp140 molecule. When bound to a given SOS gp140 molecule, the Fab arms of 2F5 and IgG1b12 lie at approximately right angles, as indicated in the schematic diagram (FIG. 18e).

In marked contrast to the IgG1b12-containing ternary complexes, those composed of SOS, 2F5 and 2G12 formed extended chains rather than closed rings (FIG. 18f). These observations place the 2F5 and 2G12 epitopes at opposite ends of the SOS gp140 molecule. There was significant heterogeneity in the stoichiometry of the 2F5/2G12/SOS gp140 complexes, just one example of which is indicated in the schematic diagram.

Immunoelectron Microscopy of SOS gp140 and gp120 in Complex with sCD4 and MAb 17b.

In an effort to further characterize the topology of SOS gp140, we reacted it with MAb 17b and/or sCD4 (FIG. 19). We generated the corresponding YU2 gp120 complexes for comparison. As expected, the combination of MAb 17b plus SOS gp140 or gp120 alone did not form complexes, consistent with the need for sCD4 to induce the 17b epitope. Similarly, unremarkable complexes were obtained when sCD4 was mixed with SOS gp140 or gp120 in the absence of MAb 17b (data not shown). However, complexes with clearly defined geometry were obtained for sCD4/Env/17b (FIGS. 19a and 19b).

These complexes were composed of 17b with one or two attached SOS gp140s or gp120s, together with tangentially protruding sCD4 molecules. These complexes were then subjected to computer-assisted averaging (FIGS. 19c and 19f). The free arm and the Fc region of MAb 17b were disordered in these images due to the flexibility of the MAb, so the averaged images were masked to highlight the better-resolved sCD4, Env and 17b Fab structures (FIGS. 19d and 19g). The gp120 and SOS gp140 images were qualitatively similar, but an image subtraction of one from the other revealed the presence of additional mass on the SOS gp140 protein (arrowed in FIGS. 19d and 19e). This additional mass may represent gp41ECTO, although we cannot strictly exclude other explanations, such as differences in the primary sequence and/or glycosylation of the gp120 and SOS gp140 proteins used.

In order to orient the putative gp41ECTO moiety in relation to the remaining structures seen in the electron micrographs, the X-ray structure of the gp120 core in complex with the D1D2 domain of sCD4 and Fab 17b (Kwong, 1998) was docked, using Program 0, into the profile map obtained for the sCD4/gp120/MAb 17b complex (FIG. 19h). Given that there are differences in the gp120 (whole vs. core) and CD4 (four domain vs. two domain) molecules used for the electron microscopy and crystallization studies, there is reasonable agreement in the overall topology of the structures generated.

This agreement in structures (FIG. 19h) enabled us to position the putative gp41ECTO moiety in relation to the core gp120 structure (FIG. 20). The previously defined neutralizing, non-neutralizing, and silent faces of gp120 (Moore, 1996; and Wyatt, 1998a) are illustrated, as are the IgG1b12 (Saphire, 2001) and 2G12 (Wyatt, 1998a) epitopes. According to this model, the gp41ECTO moiety recognized by MAb 2F5 is located at ˜90B relative to the IgG1b12 epitope and ˜180B from the 2G12 epitope (FIG. 20b). This model is in broad agreement with the independently derived electron microscopy images of the complexes formed between SOS gp140 and combinations of these MAbs (FIGS. 18e and 18f). This putative placement of gp41ECTO would cause it to largely occlude the non-neutralizing face of gp120, a result that is consistent with the MAb reactivity patterns observed for SOS gp140 both here and elsewhere (Binley, 2000a).

Antigenic Properties of Unpurified SOS gp140 and gp140UNC Proteins

Radioimmunoprecipitation assays (RIPA) was used to determine whether the antigenicity of HIV-1_{JR-FL} SOS gp140 differed when the protein was expressed in stably transfected CHO cells, compared to what was observed previously when the same protein was expressed in transiently transfected 293T cells (Binley, 2000a). The SOS gp140 proteins in unpurified supernatants expressed from CHO cells were efficiently recognized by neutralizing agents to gp120 epitopes located in the C3/V4 region (MAb 2G12), the CD4 binding site (the CD4-IgG2 molecule), and the V3 loop (MAb 19b) (FIG. 21). In addition, the conserved CD4-induced neutralization epitope defined by MAb 17b was strongly induced on SOS gp140 by sCD4. SOS gp140 was also efficiently immunoprecipitated by the broadly neutralizing gp41 MAb 2F5. In contrast, SOS gp140 was largely unreactive with the non-neutralizing MAbs 23A and 2.2B to gp120 and gp41, respectively (FIG. 21, Lanes 3 and 9). A comparison of these analyses with our previous observations (Binley, 2000a) indicates that CHO and 293T cell-derived HIV-1_{JR-FL} SOS gp140 proteins possess similar antigenic properties.

Relatively minor amounts of free gp120 were observed in the unpurified SOS gp140 CHO cell supernatants (FIG. 21, Lanes 1, 5, 7, and 8). This free gp120 was preferentially recognized by MAb 23A, suggesting that its C5 epitope is largely obscured in SOS gp140 (FIG. 21, Lane 9). This is consistent with the electron microscopy-derived topology model described above (FIG. 20b), and with what is known about the gp120-gp41 interface (Helseth, 1991; Moore, 1996; and Wyatt, 1997). Processing of SOS gp140 at the gp120-gp41 cleavage site was efficient, as determined by RIPAs performed under reducing and non-reducing conditions (FIG. 21, compare Lanes 1 and 2). Similar levels of assembly and proteolytic processing were observed when unpurified SOS gp140 was analyzed by Western blotting rather than RIPA (data not shown). These findings also are comparable to those seen with 293T cell-derived HIV-1_{JR-FL} SOS gp140 (Binley, 2000a). Thus the folding, assembly, and processing of this protein appear to be largely independent of the cell line used for its production.

Surface plasmon resonance assays SPR was used to further characterize the antibody and receptor-binding properties of unpurified, CHO cell-expressed SOS gp140 and gp140UNC proteins. A comparison of results obtained using SPR and RIPA with the same MAbs allows us to determine if the antigenicity of these proteins is method-dependent. Whereas SPR is a kinetically-limited procedure that is completed in one or more minutes, RIPA is an equilibrium method in which Env-MAb binding occurs over several hours. SPR analysis was also performed on purified and unpurified forms of the SOS gp140 and gp140UNC proteins, to assess whether protein antigenicity was significantly altered during purification. Purified HIV-1_{JR-FL} gp120 was also studied. Although the purified SOS gp140 protein is a monomer, it does contain the gp120 subunit linked to the ectodomain of gp41. Since there is evidence that the presence of gp41 can affect the antigenic structure of gp120 (Klasse, 1993; and Reitz, 1988), we thought it worth determining whether monomeric SOS gp140 behaved differently than monomeric gp120 in its interactions with neutralizing and non-neutralizing MAbs.

There was good concordance of results between RIPA- (FIG. 21) and SPR-based (FIG. 22) antigenicity analyses of unpurified SOS gp140 in CHO cell supernatants. For example, SOS gp140 bound the broadly neutralizing anti-gp41 MAb 2F5 (FIG. 21, Lane 4 and FIG. 22b) but not the non-neutralizing anti-gp41 MAb 2.2B (FIG. 21, Lane 3 and FIG. 22d). Similarly, binding of MAb 17b was strongly potentiated by sCD4 (FIG. 21, Lanes 6-7 and FIG. 22f) Unpurified SOS gp140 bound the neutralizing anti-gp120 MAbs 2G12 and 19b, but not the non-neutralizing anti-gp120 MAb 23A in both SPR (data not shown) and RIPA (FIG. 21, Lanes 1, 8, and 9) experiments. Taken together, the RIPA and SPR data indicate that unpurified, CHO cell-derived SOS gp140 rapidly and avidly binds neutralizing anti-gp120 and anti-gp41 MAbs, whereas binding to the present set of non-neutralizing MAbs is not measurable by either technique.

SPR revealed some significant differences in the reactivities of SOS gp140 and gp140UNC proteins with anti-gp41 MAbs. Thus, SOS gp140 but not gp140UNC bound MAb 2F5 but not MAb 2.2B, whereas the converse was true for gp140UNC. Notable, albeit less dramatic, differences were observed in the reactivity of SOS gp140 and gp140UNC with some anti-gp120 MAbs. Of the two proteins, SOS gp140 had the greater kinetics and magnitude of binding to the neutralizing MAbs IgG1b12 (FIG. 22g), 2G12 (FIG. 22h) and 22b in the presence of sCD4 (FIG. 22e, and 22f). The binding of gp140UNC to 17b was clearly potentiated by sCD4, as has been reported elsewhere (Zhang, 2001). Neither SOS gp140 nor gp140UNC bound the anti-gp120 MAb 23A (data not shown). This was expected for gp140UNC since the C5 amino acid substitutions that eliminate the cleavage site directly affect the epitope for MAb 23A (Moore, 1994b).

Qualitatively, the antigenicities of SOS gp140 and gp140UNC were little changed upon purification (FIG. 22, compare Panels a, c and e with Panels b, d and f). Hence the lectin affinity and gel filtration columns used for purification do not appear to significantly affect, or select for, a particular conformational state of these proteins. However, these studies do not allow for direct, quantitative comparisons of SPR data derived using purified and unpurified materials.

Compared with monomeric gp120, the purified gp140UNC protein reacted more strongly with MAb 2G12 but less strongly with MAb IgG1b12. Prior SPR studies have demonstrated that 2G12 avidly binds to oligomeric forms of Env, and it is possible that MAb 2G12 is capable of undergoing bivalent binding to oligomeric Envs. It will be informative to perform electron microscopy analyses of 2G12 in complex with gp140UNC or other oligomeric Env in future studies, given the unusual nature of the 2G12-SOS gp140 complex (FIG. 18d).

Oligomeric Properties of Unpurified SOS gp140 and gp140UNC Proteins

BN-PAGE was used to examine the oligomeric state of the SOS gp140 and gp140UNC proteins present in freshly prepared, CHO cell culture supernatants. The SOS gp140 protein was largely monomeric by BN-PAGE, with only a minor proportion of higher order proteins present (FIG. 23a). In some, but not all, 293T cell preparations, greater but highly variable amounts of dimers and higher-order oligomers were observed using BN-PAGE (data not shown, but see FIG. 23b below). This probably accounts for our previous report that oligomers can be observed in unpurified SOS gp140 preparations using other techniques (Binley, 2000a).

The unpurified gp140UNC protein typically migrated as well-resolved dimers, trimers and tetramers, with trace amounts of monomer sometimes present (FIG. 23a). Qualitatively similar banding patterns were observed for purified (FIG. 17d) and unpurified gp140UNC proteins (FIG. 23a). In each case, dimers of gp140UNC were the most abundant oligomeric species. HIV-1_{JR-FL} gp120 ran as a predominant 120 kDa monomeric band, although small amounts of gp120 dimers were observed in some unpurified supernatants. In general, the BN-PAGE analyses indicate that the oligomeric properties of the various Env proteins did not change appreciably upon purification (compare FIG. 23a and FIG. 17d).

The same CHO cell supernatants were also analyzed by analytical gel filtration, the column fractions being collected in 0.2 mL increments and analyzed for Env content by Western blotting. The retention times of unpurified gp120, SOS gp140 and gp140UNC proteins were determined to be ˜6.1, ˜5.9 and ˜5.2 minutes, respectively (data not shown). These values agree with those observed for the purified proteins (FIG. 17b) to within the precision of the method. The gel filtration studies thus corroborate the BN-PAGE data in that unpurified gp120 and SOS gp140 were mostly monomeric, while gp140UNC was mostly oligomeric (data not shown). However, unlike BN-PAGE, this analytical gel filtration procedure does not have sufficient resolving power to characterize the distribution of the oligomeric species present in the gp140UNC preparation.

SDS-PAGE followed by Western blot analyses of supernatants containing unpurified SOS gp140 and gp140UNC proteins yielded banding patterns similar to those shown in FIG. 16 for the purified proteins (data not shown). The gp120 preparation contained ˜10% dimer, which was observed only when SDS-PAGE analyses were carried out under non-reducing conditions. Thus the gp120 dimer represents disulfide-linked and presumably misfolded material (Owens, 1999).

Variable Loop-Deleted SOS gp140 Glycoproteins Form More Stable Oligomers

We previously described HIV-1_{JR-FL} SOS gp140 glycoproteins from which one or more of the gp120 variable loops were deleted to better expose underlying, conserved regions around the CD4− and coreceptor-binding sites. It was possible to remove the V1, V2 and V3 loop structures individually or in pairs without adversely affecting the formation of the intersubunit disulfide bond, proper proteolytic cleavage, or protein folding. However, the triple loop-deletant was not efficiently cleaved (Sanders, 2000). In order to explore the oligomeric properties of these modified SOS gp140 glycoproteins, the supernatants of 293T cells transiently co-transfected with these gp140 constructs and furin were analyzed by BN-PAGE. Unexpectedly, deletion of the variable loops, both alone and in combination, significantly enhanced the stability of the SOS gp140 oligomers. The ΔV1V2 SOS gp140 preparation contained almost exclusively trimeric and tetrameric species, whereas ΔV1 SOS gp140 formed a mixture of dimers, trimers and tetramers similar to that seen with gp140UNC (data not shown). The ΔV2 SOS gp140 protein was predominantly oligomeric, but it also contained significant quantities of monomer. Thus, in terms of oligomeric stability, the SOS proteins can be ranked as follows: ΔV1V2 SOS gp140>ΔV1 SOS gp140>ΔV2 SOS gp140>full-length SOS gp140. The reasons for this rank order are not yet clear, but are under investigation.

Based on the above observations, we chose to generate a CHO cell line that stably expresses the ΔV1V2 SOS gp140 protein. Supernatants from the optimized CHO cell line were first analyzed by SDS-PAGE under reducing and non-reducing conditions, followed by Western blot detection. The major Env band was seen at 120 kDa (ΔV1V2 gp140 protein) in the non-reduced gel and at 100 kDa (ΔV1V2 gp120 protein) in the reduced gel (data not shown). These results are consistent with our prior findings that deletion of the V1V2 loops decreases the apparent molecular weight of the protein by ˜20 kDa. Notably, the ΔV1V2 SOS gp140 protein was largely free both of disulfide-linked aggregates and of the ˜100 kDa loop-deleted, free gp120 protein. Thus proteolytic cleavage and SOS disulfide bond formation occur efficiently in the ΔV1V2 SOS gp140 protein (data not shown).

CHO cell supernatants containing ΔV1V2 SOS gp140, full-length SOS gp140 and gp140UNC were also analyzed by BN-PAGE and Western blotting (FIG. 23a). As was observed with the transiently transfected 293T cells, unpurified CHO cell-derived material was oligomeric. The CHO cell-derived ΔV1V2 SOS gp140 migrated as a distinct single band with a molecular weight consistent with that of a trimer (360 kDa); the ΔV1V2 SOS gp140 band lies between those of gp140UNC dimer (280 kDa) and gp140UNC trimer (420 kDa) (FIG. 23a). Hence the ΔV1V2 SOS gp140 protein represents a proteolytically mature form of HIV-1 Env that oligomerizes into presumptive trimers via non-covalent interactions. Purification and additional biophysical studies of this protein are now in progress, and immunogenicity studies are planned.

The Uncleaved SOS gp140 and gp140UNC Proteins Possess Similar Oligomeric Properties

Overall, the above analyses reveal a clear difference in the oligomeric properties of the SOS gp140 and gp140UNC proteins. One structural difference between these proteins is their proteolytic cleavage status, another is the presence or absence of the intersubunit disulfide bond that defines SOS gp140 proteins. To address the question of whether it is gp120-gp41 cleavage or the introduced cysteine residues that destabilize the SOS gp140 oligomers, we made the SOS gp140UNC protein. Here, the cysteines capable of intersubunit disulfide bond formation are present, but the cleavage site between gp120 and gp41ECTO has also been modified to prevent cleavage. The SOS gp140UNC, SOS gp140 and gp140UNC proteins were all expressed transiently in 293T cells and analyzed by BN-PAGE (FIG. 23b). In this and multiple repeat experiments, SOS gp140UNC and gp140UNC had similar migration patterns on the native gel, with the dimer band predominating and some monomers, trimers and tetramers also present. In contrast, SOS gp140 was primarily monomeric, although small amounts of dimeric and trimeric species were also observed in this particular analysis (FIG. 23b).

The above results suggest that the SOS gp140UNC protein behaves more like the gp140UNC protein than the SOS gp140 protein. This, in turn, implies that the cleavage of gp140 into gp120 and gp41ECTO has a substantial effect on how gp140 is oligomerized via interactions between the gp41ECTO moieties, whereas the presence of the cysteine substitutions in gp120 and gp41 has little effect on these interactions. We believe that this observation is central to understanding the relative instability of SOS gp140 oligomers, compared to those of the gp140UNC protein. We note, however, that we have not determined whether or not the intermolecular disulfide bond actually forms in SOS gp140UNC; the simple method of DTT treatment to reduce this bond is inadequate, because the uncleaved peptide bond between the gp120 and gp41ECTO moieties still holds the two subunits together. To address this issue will require characterizing purified SOS gp140UNC by methods such as peptide mapping. Such studies are now in progress, to further explore the effect of gp140 cleavage on the structure of the gp120-gp41ECTO complex.

E. Discussion

We have previously described the antigenic properties of SOS gp140, an HIV-1 envelope glycoprotein variant in which an intermolecular disulfide bond has been introduced to covalently link the gp120 and gp41ECTO subunits (Binley, 2000a; and Sanders, 2000). In the original report, we demonstrated that the SOS gp140 protein, as contained in supernatants of transiently transfected 293T cells, was an antigenic mimic of virion-associated Env (Binley, 2000a). In that report, the methods employed were not sufficiently robust to conclusively determine the oligomeric state of unpurified 293T-derived SOS gp140 (Binley, 2000a). Here we show that purified and unpurified CHO cell-derived SOS gp140 proteins also mimic native Env in terms of their patterns of antibody reactivity. However, unlike virus-associated Env, SOS gp140 is a monomeric protein.

Antigenicity and immunoelectron microscopy studies support a model for SOS gp140 in which the neutralizing face of gp120 is presented in a native conformation, but the non-neutralizing face is occluded by gp41ECTO. The immunoelectron microscopy data suggest a model in which the gp41ECTO moiety of SOS gp140 occludes the non-neutralizing face of the gp120 subunit (FIG. 20). The evidence for this model is derived from several independent studies. In the first of these, SOS gp140 was examined in complex with combinations of anti-gp120 and anti-gp41 MAbs to defined epitopes (FIG. 18). The gp41ECTO subunit, as defined by the position of the anti-gp41 MAb 2F5, was located ˜180B from the MAb 2G12 epitope and ˜90B from the MAb IgG1b12 epitope, as is the non-neutralizing face. A second set of studies compared SOS gp140 and gp120 in complex with sCD4 and MAb 17b (FIG. 19). Here, a region of additional mass in the gp140 complex defined the presumptive gp41ECTO; its location was similarly adjacent to the non-neutralizing face of gp120. This model of the geometry of the gp120-gp41 interaction is consistent with previous models based on mutagenesis techniques and the mapping of MAb epitopes (Helseth, 1991); Moore, 1996; and Wyatt, 1997). It also provides a basis for interpreting the patterns of MAb reactivity described above and discussed below.

The antigenicity of CHO-derived SOS gp140 was explored from a number of perspectives: (1) in comparison with gp140UNC and gp120; (2) before and after purification; (3) in an equilibrium-based assay (RIPA) vs. a kinetics-based assay (SPR). SOS gp140 proteins expressed in stably transfected CHO cells or transiently transfected 293T cells possessed qualitatively similar antigenic properties that were largely unaffected by purification. We observed that most neutralizing anti-gp120 MAbs bound more strongly and more rapidly to SOS gp140 than to the gp120 or gp140UNC proteins, whereas the converse was true of non-neutralizing MAbs (FIGS. 21 and 22). These results were largely independent of the analytical methodology used (RIPA or SPR), or the purification state of the glycoproteins, and thus extend our earlier studies on the antigenicity of unpurified Env glycoproteins determined by RIPA (Binley, 2000a). We have addressed these issues on a largely qualitative basis in the present study; quantitative comparisons of MAb reactivities are now being explored.

It is not obvious why neutralizing MAbs recognize monomeric SOS gp140 better than monomeric gp120. One possibility relates to differences in the conformational freedom of the two glycoproteins. Monomeric gp120 has considerable conformational flexibility, such that Afreezing@ of the conformation by CD4 binding results in an unexpectedly large loss in entropy (Myszka, 2000). Indeed, it has been suggested that reducing the conformational freedom of a gp120 immunogen may provide a means of generating broadly neutralizing antibodies, which generally recognize conformational epitopes (Myszka, 2000). The presence of gp41ECTO may serve to minimize the conformational flexibility of the gp120 subunit of SOS gp140, stabilizing the protein in conformations recognized by neutralizing antibodies. However, the induction of 17b binding by sCD4 demonstrates that SOS gp140 is still capable of sampling multiple, relevant conformations. Studies are in progress to address these issues.

Variations in conformational flexibility may also underlie the antigenic differences observed between the SOS gp140 and gp140UNC proteins. Other possible explanations include the effect that cleavage may have on the overall structure of Env, and differences in the oligomerization state of the two proteins. Further studies using additional Env protein variants (e.g., SOS gp140UNC), a broader range of anti-Env MAbs, and purified or size-fractionated proteins of a homogenous subunit composition, will be required to explore these issues more thoroughly.

Standard biophysical techniques were used to demonstrate that the purified HIV-1_{JR-FL} SOS gp140 glycoprotein is a monomer comprising one gp120 subunit disulfide-linked to gp41ECTO. Since it is generally accepted that the gp41 subunits are responsible for Env trimerization (Caffrey, 1998; Chan, 1997; Lu, 1995; Tan, 1997; and Weissenhorn, 1997), we assume that the gp41-gp41 interactions within the cleaved SOS gp140 glycoprotein are weak, and that this instability precludes the purification of cleaved trimers.

We also report the application of a rapid, simple and high-resolution electrophoretic technique, BN-PAGE, for exploring the oligomeric state of HIV-1 envelope glycoproteins in unpurified as well as purified form. In this technique, the proteins of interest are combined with the dye coomassie blue, which binds to the exposed hydrophobic surfaces of proteins and usually enhances their solubility. In the presence of the dye, most proteins adopt a negative charge, migrate towards the anode in an electric field, and so can be sieved according to their Stokes=radius in a polyacrylamide gradient gel. Whereas traditional native PAGE methods are typically performed under alkaline conditions (pH 9.5), BN-PAGE uses a physiological pH (pH 7.5), which is more compatible with protein stability. We demonstrate that a gp120/sCD4 complex and a variety of purified, oligomeric model proteins all remain associated during BN-PAGE analysis. When combined with Western blot detection, BN-PAGE can be used to determine the oligomeric state of HIV-1 envelope glycoproteins at all stages of purification. This high resolution technique can resolve monomeric, dimeric, trimeric and tetrameric forms of gp140.

As determined by BN-PAGE and other methods, the SOS gp140 protein was secreted in mostly monomeric form. In contrast, gp140UNC proteins, in which the peptide bond between gp120 and gp41 still attaches the two subunits, form oligomers that are significantly more stable. Thus, we show that HIV-1_{JR-FL} gp140UNC comprises a mixture of dimers, trimers and tetramers, with dimers representing the major oligomeric form present under non-denaturing conditions. Although non-covalently associated oligomers constitute a significant percentage of the gp140UNC preparation, half or more of the material consists of disulfide-linked and presumably misfolded material (Owens, 1999). Others have made similar observations with uncleaved gp140 proteins from other HIV-1 strains, and from SIV (Chen, 2000; Earl, 1997; Earl, 1994; Earl, 1990; Earl, 2001; Edinger, 2000; Farzan, 1998; Hoffman, 2000; Owens, 1999; Richardson, 1996; Stamatatos, 2000; Staropoli, 2000; Yang, 2000a; Yang, 2000b); and Yang, 2001). The question then arises as to why the SOS gp140 protein is a monomer, but the uncleaved proteins are oligomeric. We believe that the cleavage of the gp120-gp41 peptide bond alters the overall conformation of the envelope glycoprotein complex, rendering it fusion-competent but also destabilizing the association between the gp41 subunits. Support for this argument is provided by the evidence that the SOS gp140UNC protein behaves identically to the gp140UNC protein, but very differently from the SOS gp140 protein; cleavage is clearly more important than the engineered, intermolecular disulfide bind in determining the oligomeric stability of gp140 proteins. Destabilization of gp41-gp41 interactions might be necessary for gp41-mediated fusion to occur efficiently upon activation of the Env complex by gp120-receptor interactions. Moreover, having cleavage/activation take place late in the synthetic process minimizes the risk of fusion events occurring prematurely, i.e. during intracellular transport of the envelope glycoprotein complex. Additional studies are in progress to explore the effect of cleavage on Env structure.

Taken together, the antigenic and biophysical data of SOS gp140, gp120 and gp140UNC suggest that SOS gp140 represents an improved yet clearly imperfect mimic of native Env. It is perhaps surprising that an SOS gp140 monomer mimics virus-associated Env in its reactivity with a diverse panel of MAbs. Immunochemical studies and the X-ray crystal structure of the gp120 core in complex with CD4 and MAb 17b have together defined the surface of gp120 in terms of neutralizing, non-neutralizing and silent faces (Kwong, 1998; and Wyatt, 1998a). The data presented here and elsewhere (Binley, 2000a) demonstrate the neutralizing face is readily accessible on SOS gp140, whereas the non-neutralizing face is not. There are still no immunologic ways to probe the exposure of the silent face of gp120 (Moore, 1996). A source of purified SOS gp140 glycoprotein, as described herein, will facilitate further studies of the antigenic structure of SOS gp140 in comparison with that of native Env.

Do gp140UNC proteins mimic the structure of the native, fusion-competent envelope glycoprotein complex on virions? We believe not, based on their exposure of non-neutralizing epitopes in both gp120 and gp41 that are not accessible on the surface of native envelope glycoprotein complexes (Binley, 2000a; and Sattentau, 1995). Neutralization epitopes overlapping the CD4 binding site are poorly presented on HIV-1_{BH8} gp140UNC relative to virus-associated Env (Parren, 1996), and only one CD4 molecule can bind to the SIVmac32H gp140UNC protein. The lack of correlation between the binding of MAbs to uncleaved envelope glycoprotein complexes on the surface of Env-transfected cells and neutralization of the corresponding viruses again argues that uncleaved complexes have an abnormal configuration (York, 2001). However, in the absence of definitive and comparative structural information on native and uncleaved Env complexes, this is an unresolved point. At present it is not possible to predict what antigenic structures will elicit a desired immune response; that can only be defined empirically, and it may be that one or more uncleaved forms of Env will be effective immunogens even if they do not properly mimic the structure of the native Env complex. Given this situation, we believe it is relevant to design and rigorously test different Envs, such as SOS gp140, that possess distinct antigenic properties.

Given that SOS gp140 is monomeric, what can be done to further stabilize the structure of fully cleaved, envelope glycoprotein complexes? The immunoelectron microscopy data of the 2G12/SOS gp140 complex suggest that appropriately directed antibodies could strengthen weak oligomeric interactions. The immunogenicity of such complexes may be worth testing, although a bivalent MAb might be expected to promote formation of Env dimers rather than trimers. We have already attempted to combine the SOS gp140 disulfide bond stabilization strategy with one in which the gp41 subunits were also stabilized by an intermolecular disulfide bond B this was unsuccessful, in that the mutated protein was poorly expressed and could not be cleaved into gp120 and gp41 subunits, even in the presence of co-transfected furin. Similarly, adding GCN-4 domains onto the C-terminus of gp41 hindered the proper cleavage of gp140 into gp120 and gp41 furin. Other approaches, based on site-directed mutagenesis of selected gp41 residues, are presently being evaluated.

Fortuitously, we have found that variable-loop-deleted forms of HIV-1_{JR-FL} SOS gp140 form more stable oligomers than their full-length counterparts. Thus, the SOS gp140 proteins lacking either the V1 or V2 variable loops contain a greater proportion of oligomers than the full-length protein, and the V1V2 double loop-deletant is expressed primarily as noncovalently-associated trimers. One hypothesis is that the extended and extensively glycosylated variable loops sterically impede the formation of stable gp41-gp41 interactions in the context of the full-length SOS gp140 protein. Indeed, using the crystal structure of the gp120/CD4/17b complex, Kwong et al. have developed a model of oligomeric gp120 that places V1V2 sequences at the trimer interface (Kwong, 2000). The variable-loop-deleted SOS gp140 proteins may therefore represent proteolytically mature HIV-1 envelope glycoproteins that can perhaps eventually be produced and purified as oligomers. We previously demonstrated that unpurified forms of variable-loop-deleted SOS gp140 proteins possess favorable antigenic properties (Sanders, 2000). These proteins are therefore worth further evaluation in structural and immunogenicity studies.

### Experimental Set III—Particle Vaccines

A. Materials and Methods

Antibodies and recombinant HIV-1 envelope antigen. The expression vectors designated CD4-IgG2HC-pRcCMV and CD4-kLC-pRcCMV were deposited pursuant to, and in satisfaction of, the requirements of the Budapest Treaty with ATCC under ATCC Accession Nos. 75193 and 75104. CD4-IgG2 protein was produced in purified form as described from Chinese hamster ovary cells stably co-transfected with CD4-IgG2HC-pRcCMV and CD4-kLC-pRcCMV (Allaway, 1995).

The expression vector designated PPI4-tPA-gp120_{JR-FL} was deposited pursuant to, and in satisfaction of, the requirements of the Budapest Treaty with ATCC under ATCC Accession Number. 75432. Recombinant HIV-1_{JR-FL} gp120 protein was produced in purified form as described from Chinese hamster ovary cells stably co-transfected with PPI4-tPA-gp120JR-FL as described previously (U.S. Pat. No. 5,869,624).

A hybridoma cell line secreting the mouse monoclonal antibody (PA1) to the V3 loop of HIV-1_{JR-FL} gp120 was prepared.

The human monoclonal antibody IgG1b12 (National Institutes of Health AIDS Research and Reference Reagent Program [ARRRP] Cat. Number 2640) binds an epitope on gp120 that overlaps the CD4 binding site (Burton, 1991). The human monoclonal antibody 2G12 (ARRRP Cat. Number 1476) binds a glycan-dependent epitope on gp120 (Trkola, 1996b). The human monoclonal antibody 2F5 (ARRRP Cat. Number 1475) binds the HIV-1 envelope transmembrane glycoprotein gp41 (Muster, 1993).

Preparation of Miltenyi μ-MACS Protein G Microbeads. 2 mg of purified PA1 (1 mg/ml) were incubated overnight with 4 ml of a suspension of Miltenyi μ-MACS Protein G microbeads (Miltenyi Biotec; Cat. Number 130-071-101) at 4° C. The next day the microbeads were pelleted in a Sorvall RC5C ultracentrifuge (SS-34 rotor) at 12,000 rpm (˜20,000×g) for 15 minutes. The isolated microbeads were washed once with 400 μl PBS and pelleted in a microcentrifuge at 15,000 rpm (˜16,000×g) for 15 minutes. If protein was to be immunoprecipitated with the mAb bound to the beads, the beads were resuspended with the protein solution (see below). Otherwise, the PA1-beads were gently resuspended in PBS at a concentration of 1 mg/ml PA1. Using this method, ˜150 μg of PA1 could be immobilized per ml of microbead suspension.

The immobilization of CD4-IgG2, 2G12, IgG1b12, and 2F5 was performed essentially as described for PA1. The capacity was ˜40 μg of CD4-IgG2, ˜55 μg of IgG1b12, ˜60 μg of 2G12, or ˜95 μg of 2F5 per ml of microbead suspension.

Capture of HIV-1_{JR-FL} gp120 onto PA1 microbeads. Beads containing 600 μg of PA1 were carefully resuspended with 3 mg of HIV-1_{JR-FL} gp120 and incubated overnight at 4° C. The efficiency of gp120 binding to the PA1-beads was increased when the incubation was performed over 3 days. Following the capture of gp120, the microbeads were pelleted in a Sorvall RC5C ultracentrifuge (SS-34 rotor) at 12,000 rpm (˜20,000×g) for 15 minutes. The isolated microbeads were washed once with 400 μl PBS and pelleted in a microcentrifuge at 15,000 rpm (˜16,000×g) for 15 minutes. Subsequent to the wash the gp120-loaded beads were thoroughly resuspended with PBS at a concentration of 1 mg/ml gp120 (as determined by SDS-PAGE and Coomassie staining of the protein bands). Using this method, ˜800 μg of gp120 were routinely immobilized with 600 μg of PA1 (FIG. 24). Efficient capture of antigen was obtained using both purified gp120 in PBS buffer and gp120 in cell culture media (Sigma Chemical Company, St. Louis, Mo., Cat. Number C1707) containing 1% L-glutamine (Life Technologies, Gaithersburg, Md., Cat. Number 25030-081) and 0.02% bovine serum albumin (Sigma Cat. Number A7409).

Immobilization of antibody onto Dynabeads® Protein G. The PA1 antibody was produced as described above. 0.5 mg of purified PA1 (1 mg/ml) were incubated overnight with 0.1 ml of a suspension of Dynabeads Protein G (Dynal Biotech Inc., Cat. Number 100.04) at 4° C. The next day the Dynabeads were collected with a magnet (Dynal Magnetic Particle Concentrator, Dynal MPC®) and washed once with PBS. If protein was to be immunoprecipitated with the mAb bound to the beads, the Dynabeads were resuspended directly with the protein solution (see below). Otherwise, the PA1-beads were carefully resuspended in PBS at a concentration of 1 mg/ml PA1. Using this method, ˜150 μg of PA1 could be immobilized per ml of Dynabeads suspension (FIG. 25).

Capture of HIV-1_{JR-FL} gp120 onto Dynabeads. Beads containing 15 μg of PA1 were gently resuspended with 200 μg of HIV-1_{JR-FL} gp120 and incubated overnight at 4° C. Following capture, the gp120-loaded Dynabeads were collected with a magnet and washed twice with PBS. Subsequent to the wash steps, the gp120-beads were carefully resuspended in PBS at a concentration of 1 mg/ml gp120. Using this method, ˜3.3 μg of gp120 were routinely immobilized with 15 μg of PA1.

SDS-PAGE analysis of biospecific bead vaccines. HIV-1_{JR-FL} gp120 immobilized to the beads was analyzed by SDS-PAGE as follows: 20 μl of resuspended beads were mixed with the same volume of 2×LDS/DTT sample buffer (140 mM Tris Base, 106 mM Tris/HCl, 2% SDS, 10% glycerol, 25 mM DTT, 0.5 mM EDTA, pH 8.5) and incubated at 70° C. for 5 minutes. 10 μl and 25 μl of the Miltenyi microbeads samples or 2 μl, 5 μl, 10 μl, and 20 μl of the Dynabeads samples were loaded onto a 4-12% NuPAGE Bis-Tris gel (Invitrogen) and electrophoresed at 175V for 50 minutes using the MES/SDS running buffer system (50 mM MES, 50 mM Tris Base, 0.1% SDS, 1 mM EDTA, pH 7.7). Included in the gels were known concentrations of gp120 treated as described for the beads for quantitation purposes. Following electrophoresis, the gels were fixed in 10% acetic acid/40% methanol and subsequently stained according to the manufacturers' protocol using the Gelcode Blue staining solution (Pierce). The stained protein bands were analyzed and quantitated by densitometry (Molecular Dynamics).

Immunization of mice with biospecific bead vaccines. Successful vaccination relies on the induction of a protective immune response to an antigen of interest. Effective presentation of antigen to the immune system can be achieved by delivery of highly purified protein with an immunostimulatory adjuvant. We describe a novel dual-purpose approach using magnetic beads that (1) enables efficient purification of antigen for immunization and (2) enhanced immune responses to the antigen in animals.

Immunogens. Purified gp120 (Subtype B, JR-FL; 1 mg/ml) was used at the indicated doses. Gp120 was admixed with the adjuvant, QS-21 (10 μg per dose; Antigenics), or captured on Miltenyi MACS magnetic beads by the anti-gp120 mAb, PA1 as described above. Groups of animals received beads either with or without QS-21.

Immunizations. Groups of 5 female Balb/C mice (6-10 weeks of age at the onset of studies; Charles River Laboratories, Boston, Mass.) were used for each vaccine. Three immunizations were administered in 200 μl volume at 2-week intervals by subcutaneous injection in the flank-region using ½ cc insulin syringes and 28G gauge needles (Becton Dickinson, Franklin Lakes, N.J.).

Sera and tissue collection. Mice were bled through the retro orbital plexus one day prior to each immunization, and the sera separated by centrifugation in blood-collection Capiject tubes (Terumo; Somerset, N.J.). Aliquots of the separated sera were cryopreserved at −80° C. before analysis.

Spleens were harvested and pooled from the 5 mice per group and single cell suspensions prepared by gently teasing the tissue through a 70 μm nylon mesh filter. Cells were cryopreserved at −196° C. before analysis.

ELISA assay. HIV-1 gp120 specific antibodies in sera were quantified by a standard ELISA assay (Binley, 1997). Briefly, 96-well ELISA plates were coated with HIV-1_{JR-FL} gp120 via adsorbed sheep anti-gp120 mAb D7324 (Aalto BioReagents, Dublin, Ireland) and blocked before addition of serial dilutions of serum samples from individual mice in triplicate wells. After incubation, the wells were washed and incubated with a dilution of anti-mouse IgG-detection antibody conjugate before addition of chromogenic substrate. Binding was measured using an ELISA plate reader at OD490. Titers (50% maximal) were calculated for each group as defined by the antibody dilution giving half-maximal binding after background subtraction (wells with no antigen). The mean values +/− SD of replicate wells are represented.

ELISPOT assay. HIV-gp120 specific T cells are quantified using an IFNγ-ELISPOT assay, essentially as described (Miyahira, 1995). Briefly, mixed cellulose ester membrane 96-well plates (Millipore) are coated with an anti-mouse anti-IFNγ antibody (5 μg/ml; MABTech) for 2 hours at 37° C. and washed thrice in PBS. The wells are blocked in complete RPMI medium (RPMI 1640, α-MEM, FBS (10%, Gibco) HEPES (10 mM Gibco), L-Gln (2 mM), 2-mercaptoethanol (50 μM) for a further 2 hours at 37° C. After washing the wells thrice with PBS, single cell suspensions of splenocytes are added at 1-5×10⁵ cells per well in the presence of gp120 protein (5 μM) or H-2^{d} restricted gp120 peptide (RGPGRAFVTI (2 μM)) for 16-20 hours. Plates are washed extensively in PBS/Tween-20 (PBS-T; 0.05%) and incubated for 1 hour with biotinylated anti-IFNγ antibody (2 μg/ml; MABTech) at room temperature. The plates are washed thrice in PBS/T and incubated for 2 hours with streptavidin-HRP (Vectastain Elite ABC Kit). After washing with PBS/T, the HRP-substrate, AEC (3-amino-9-ethylcarbazole; Sigma), is added for 15 minutes at room temperature. The reaction is stopped by added de-ionized water, and the wells are washed before drying in air for 24 hours. The spots are enumerated using an automatic ELISPOT plate reader (Carl Zeiss, Germany) and software. Each condition is performed in triplicate with serial dilutions of splenocytes and the frequency of spot-forming cells (SFCs) per 106 splenocytes is calculated. Negative controls samples with splenocytes and complete medium alone are used to determine background levels, and a positive signal is defined as >2-fold SFCs in control wells.

B. Results

The ability of magnetic beads to potentiate immune responses to an antigen of interest was examined using HIV-1 envelope protein, gp120, attached to magnetic beads with an anti-gp120-specific antibody (PA1). Preparations of beads were administered thrice subcutaneously to groups of mice to achieve a gp120 dose of 25 μg or 5 μg, with or without the immunostimulatory adjuvant, QS-21. Control groups of animals received gp120 (25 μg or 5 μg) with QS-21, gp120 admixed with QS21 and PA1 (no beads), or magnetic beads with PA1 (no gp120). Sequential bleeds after each dose were performed and serum separated for analysis of the anti-gp120 humoral response with a standard ELISA assay. Temporal analysis of sera demonstrated that immune responses increased after each immunization, and that anti-gp120 antibody titers were maximal after three doses (FIG. 26). The titers of antibodies were measured with serial dilutions of the sera, and indicated that bead-captured gp120 with QS-21 was the most potent immunogen (FIG. 27). This response was correlated with the dose of gp120, and animals immunized with 25 μg gp120 had higher levels of serum antibodies. Importantly, these responses were approximately one order of magnitude greater than those in animals receiving gp120 and QS21 without beads. These data indicate that magnetic beads augment the immune response to captured antigen, and this technology may have utility for vaccine development

### REFERENCES

1. Allaway, G. P., Davis-Bruno K. L., Beaudry G. A., Garcia E. B., Wong E. L., Ryder A. M., Hasel K. W., Gauduin M. C., Koup R. A., McDougal J. S., and Maddon P. J. (1995) Expression and characterization of CD4-IgG2, a novel heterotetramer which neutralizes primary HIV-1 isolates. AIDS Res. Hum. Retroviruses. 11:533-539.
2. Allaway, G. P., Ryder A. M., Beaudry G. A., and Maddon P. J. (1993) Synergistic inhibition of HIV-1 envelope-mediated cell fusion by CD4-based molecules in combination with antibodies to gp120 or gp41. AIDS Res. Hum. Retroviruses 9:581-587.
3. Alving, C. R., Koulchin V., Glenn G. M., and Rao M. (1995) Liposomes as carriers of peptide antigens: induction of antibodies and cytotoxic T lymphocytes to conjugated and unconjugated peptides. Immunol. Rev. 145:5-31.
4. Andre, S., Seed B., Eberle J., Schraut W., Bultmann A. and Haas J. (1998) Increased immune response elicited by DNA vaccination with a synthetic gp120 sequence with optimized codon usage. J. Virol. 72:1497-503.
5. Atwell, S., Ridgway J. B., Wells J. A. and Carter P. (1997) Stable heterodimers from remodeling the domain interface of a homodimer using a phage display library. J. Mol. Biol. 270:26-35.
6. Berman, P. W., Gregory T. J., Riddle L, Nakamura G. R., Champe, M. A., Porter J. P., Wurm F. M., Hershberg R. D., Cobb E. K. and Eichberg J. W. (1990) Protection of chimpanzees from infection by HIV-1 after vaccination with recombinant glycoprotein gp120 but not gp160. Nature 345:622-625.
7. Berman, P. W., Nunes W. M. and Haffar O. K. (1988) Expression of membrane-associated and secreted variants of gp160 of human immunodeficiency virus type 1 in vitro and in continuous cell lines. J. Virol. 62:3135-3142.
8. Berman, P. W., Riddle L., Nakamura G., Haffar O. K., Nunes W. M., Skehel P., Byrn R., Groopman J., Matthews T. and Gregory T. (1989) Expression and immunogenicity of the extracellular domain of the human immunodeficiency virus type 1 envelope glycoprotein, gp160. J. Virol. 63: 3489-98.
9. Binley, J., and Moore J. P. (1997b) HIV-cell fusion. The viral mousetrap. 387:346-348.
10. Binley, J. M., Klasse P. J., Cao Y., Jones I., Markowitz M., Ho D. D., and Moore J. P. (1997a). Differential regulation of the antibody responses to Gag and Env proteins of human immunodeficiency virus type 1. J. Virol. 71:2799-2809.
11. Binley, J. M., Wyatt R., Desjardins E, Kwong P. D., Hendrickson W., Moore J. P. and Sodroski J. (1998) Analysis of the interaction of antibodies with a conserved enzymatically deglycosylated core of the HIV type 1 envelope glycoprotein 120.AIDS Res. Human Retrovir. 14:191-98.
12. Buchacher, A., Predl R., Strutzenberger K., Steinfellner W., Trkola A., Purtscher M., Gruber G., Tauer C., Steindl F., Jungbauer A., et al. (1994) AIDS Res. Human Retrov. 10:359-369.
13. Burton, D. R. and Montefiori D. C., (1997) The antibody response in HIV-1 infection. AIDS. 11 Suppl A:S87-S98.
14. Burton, D. R., Barbas C. F., Persson M. A., Koenig S., Chanock R. M., and Lerner R. A. (1991) A large array of human monoclonal antibodies to type 1 human immunodeficiency virus from combinatorial libraries of asymptomatic seropositive individuals. Proc. Nat. Acad. Sci. U.S.A. 88:10134-10137.
15. Burton, D. R., Pyati J., Koduri R., Sharp S. J., Thornton G. B, Parren P. W., Sawyer L. S., Hendry R. M., Dunlop N., Nara P. L., et al. (1994) Efficient neutralization of primary isolates of HIV-1 by a recombinant human monoclonal antibody. Science. 266:1024-1027.
16. Caffrey, M., Cai M., Kaufman J., Stahl S. J., Wingfield P. T., Covell D. G., Gronenborn A. M. snd Clore G. M. (1998) Three-dimensional solution structure of the 44 kDa ectodomain of SIV gp41. EMBO J. 17:4572-4584.
17. Cao, J., Sullivan N., Desjardin E., Parolin C., Robinson J., Wyatt R. and Sodroski J. (1997) Replication and neutralization of human immunodeficiency virus type 1 lacking the V1 and V2 variable loops of the gp120 envelope glycoprotein. J. Virol. 71: 9808-9812.
18. Chen, B., Zhou G., Kim M., Chishti Y., Hussey R. E., Ely B., Skehel J. J., Reinherz E. L., Harrison S. C. and Wiley D. C. (2000) Expression, purification, and characterization of gp160e, the soluble, trimeric ectodomain of the simian immunodeficiency virus envelope glycoprotein, gp160. J. Biol. Chem. 275:34946-34953.
19. Chen, Y. T., Scanlan M. J., Sahin U., Tureci O., Gure A. O., Tsang S., Williamson B., Stockert E., Pfreundschuh M., and Old J. J. (1997) A testicular antigen aberrantly expressed in human cancers detected by autologous antibody screening. Proc. Natl. Acad. Sci. U.S.A. 94:1914-1918.
20. Cherpelis, S., Shrivastava I., Gettie A., Jin X., Ho D. D., Barnett S. W. and Stamatatos L. (2001) DNA vaccination with the human immunodeficiency virus type 1 SF162DeltaV2 envelope elicits immune responses that offer partial protection from simian/human immunodeficiency virus infection to CD8(+) T-cell-depleted rhesus macaques. J. Virol. 75:1547-1550.
21. Cleland, J. L., Powell M. F., Lim A., Barron L., Berman P. W., Eastman D. J., Nunberg J. H., Wrin T., and Vennari J. C. (1994) Development of a single-shot subunit vaccine for HIV-1. AIDS Res. Hum. Retroviruses 10 Suppl 2:S21-26.
22. Collman, R., Balliet J. W., Gregory S. A., Friedman H., Kolson D. L., Nathanson N. and Srinivasan A. (1992) An infectious molecular clone of an unusual macrophage-tropic and highly cytopathic strain of human immunodeficiency virus type 1. J. Virol. 66: 7517-21.
23. Coombes, A. G., Lavelle E. C., and David S. S. (1999) Biodegradable lamellar particles of poly(lactide) induce sustained immune responses to a single dose of adsorbed protein. Vaccine. 2410-2422.
24. Coombes, A. G., Lavelle E. C., Jenkins P. G., and Davis S. S. (1996) Single dose, polymeric, microparticle-based vaccines: the influence of formulation conditions on the magnitude and duration of the immune response to a protein antigen. Vaccine 14:1429-1438.
25. diMarzo Veronese et al. (1992) AIDS Res. Human Retrov. 8:1125.
26. Doe, B., Selby M., Barnett S., Baenziger J. and Walker C. M. (1996) Induction of cytotoxic T lymphocytes by intramuscular immunization with plasmid DNA is facilitated by bone marrow-derived cells. Proc. Natl. Acad. Sci. U.S.A. 93(16):8578-8583.
27. Doms, R. W. and Moore J. P. (2000b) HIV-1 membrane fusion: targets of opportunity. J. Cell. Biol. 151:F9-14.
28. Earl, P. L., Doms R. W. and Moss B. (1990) Oligomeric structure of the human immunodeficiency virus type 1 envelope glycoprotein. Proc. Nat. Acad. Sci. U.S.A. 87:648-652.
29. Earl, P. L., Broder C. C., Doms R. W. and Moss B. (1997) Epitope map of human immunodeficiency virus type 1 gp41 derived from 47 monoclonal antibodies produced by immunization with oligomeric envelope protein. J. Virol. 71:2674-2684.
30. Earl, P. L., Broder C. C., Long D., Lee S. A., Peterson J., Chakrabarti S., Doms R. W. and Moss B. (1994b) Native oligomeric human immunodeficiency virus type 1 envelope glycoprotein elicits diverse monoclonal antibody reactivities. J. Virol. 68:3015-3026.
31. Edinger, A. L., Ahuja M., Sung T., Baxter K. C., Haggarty B., Doms R. W. and Hoxie J. A. (2000) Characterization and epitope mapping of neutralizing monoclonal antibodies produced by immunization with oligomeric simian immunodeficiency virus envelope protein. J. Virol. 74:7922-7935.
32. Edinger, A. L., Blanpain C., Kunstman K. J., Wolinsky S. M., Parmentier M. and Doms R. W. (1999) Functional dissection of CCR5 coreceptor function through the use of CD4-independent simian immunodeficiency virus strains. J. Virol. 73:4062-73.
33. Feltquate, D. M., Heaney S., Webster R. G. and Robinson H. L. (1997) Different T helper cell types and antibody isotypes generated by saline and gene gun DNA immunization. J. Immunol. 158(5):2278-2284.
34. Fouts, T. R., Binley J. M., Trkola A., Robinson J. E. and Moore J. P. (1997) Neutralization of the human immunodeficiency virus type 1 primary isolate JR-FL by human monoclonal antibodies correlates with antibody binding to the oligomeric form of the envelope glycoprotein complex. J Virol 71: 2779-85.
35. Fouts, T. R., Trkola A., Fung M. S and Moore J. P. (1998) Interactions of polyclonal and monoclonal anti-glycoprotein 120 antibodies with oligomeric glycoprotein 120-glycoprotein 41 complexes of a primary HIV type 1 isolate: relationship to neutralization. AIDS Res. Human Retrovir. 14: 591-597.
36. Frank, J., Radermacher M., Penczek P., Zhu J., Li Y., Ladjadj M. and Leith A. (1996) SPIDER and WEB: processing and visualization of images in 3D electron microscopy and related fields. J. Struct. Biol. 116:190-199.
37. Gallaher, W. R., Ball J. M., Garry R. F., Martin-Amedee A. M. and Montelaro R. C. (1995) A general model for the surface glycoproteins of HIV and other retroviruses. AIDS Res. Human Retrovir. 11: 191-202.
38. Gerl, M., Jaenicke R., Smith J. M. and Harrison P. M. (1988) Self-assembly of apoferritin from horse spleen after reversible chemical modification with 2,3-dimethylmaleic anhydride. Biochemistry 27:4089-4096.
39. Gershoni, J. M., Denisova G., Raviv D., Smorodinsky N. I. and Buyaner D. (1993) HIV binding to its receptor creates specific epitopes for the CD4/gp120 complex. FASEB J. 7:1185-1187.
40. Gorse, G. J., McElrath M. J., Matthews T. J., Hsieh R. H., Belshe R. B., Corey L., Frey S. E., Kennedy D. J., Walker M. C. and Eibl M. M. (1998) Modulation of immunologic responses to HIV-1MN recombinant gp160 vaccine by dose and schedule of administration. National Institute of Allergy and Infectious Diseases AIDS Vaccine Evaluation Group. Vaccine 16:493-506.
41. Haas, J., Park E. C. and Seed B. (1996) Codon usage limitation in the expression of HIV-1 envelope glycoprotein. Current Biol. 6:315-24.
42. Hanes, J., Cleland J. L. and Langer R. (1997) New advances in microsphere-based single-dose vaccines. Advanced Drug Delivery Reviews 28:97-119.
43. He, Q., Mithell A. R., Johnson S. L., Wagner-Bartak C., Morcol T. and Bell S. J. (2000) Calcium phosphate nanoparticle adjuvant. Clinical and Diagnostic Laboratory Immunology 7:899-903.
44. Helseth, E., Olshevsky U., Furman C and Sodroski J. (1991) Human immunodeficiency virus type 1 gp120 envelope glycoprotein regions important for association with the gp41 transmembrane glycoprotein. J. Virol. 65:2119-2123.
45. Hoffman, T. L., Canziani G., Jia L., Rucker J. and Doms R. W. (2000) A biosensor assay for studying ligand-membrane receptor interactions: binding of antibodies and HIV-1 Env to chemokine receptors. Proc. Natl. Acad. Sci. U.S.A. 97:11215-11220.
46. Hollstein, M., Rice K., Greenblatt M. S., Soussi T., Fuchs R., Sorlie T., Hovig E., Smith-Sorenson B., Montesano R. and Harris C. C. (1994) Database of p53 gene somatic mutations in human tumors and cell lines. Nucleic Acids Res. 22:3551-3555
47. Israeli, R. S., Powell C. T., Fair W. R. and Heston W. D. (1993) Molecular cloning of a complementary DNA encoding a prostate-specific membrane antigen. Cancer Res. 53:227-230
48. Jackson, I. J., Chambers D. M., Tsukamoto K., Copeland N. G., Gilbert D. J., Jenkins N. A., and Hearing V. (1992) A second tyrosinase-related protein, TRP-2, maps to and is mutated at the mouse slaty locus. EMBO J. 11:527-535.
49. Johnson, M. L., Correia J. J., Yphantis D. A. and Halvorson H. R. (1981) Analysis of data from the analytical ultracentrifuge by nonlinear least-squares techniques. Biophys. J. 36:575-588.
50. Jones, D. H., McBride B. W., Roff M. A. and Farrar G. H. (1995) Efficient purification and rigorous characterization of a recombinant gp120 for HIV vaccine studies. Vaccine 13:991-999.
51. Jones, P. L., Korte T. and Blumenthal R. (1998) Conformational changes in cell surface HIV-1 envelope glycoproteins are triggered by cooperation between cell surface CD4 and co-receptors. J. Biol. Chem. 273:404-409.
52. Kang, C. Y., Hariharan K., Nara P. L., Sodroski J. and Moore J. P. (1994) Immunization with a soluble CD4-gp120 complex preferentially induces neutralizing anti-human immunodeficiency virus type 1 antibodies directed to conformation-dependent epitopes of gp120. J. Virol. 68:5854-62.
53. Kawakami, Y., Eliyahu S., Delagado C. H., Robbins P. F., Sakaguchi K., E. Appella, J. R. Yannelli, G. J. Adema, and S. A. Rosenberg. (1994b) Identification of a human melanoma antigen recognized by tumor-infiltrating lymphocytes associated with in vivo tumor rejection. Proc. Natl. Acad. Sci. U.S.A. 91:6458-6462.
54. Kawakami, Y., Eliyahu S., Sakaguchi K., Robbins P. F., Rivoltini L., Yannelli J. R., Appella E., and Rosenberg S. A. (1994a) Identification of the immunodominant peptides of the MART-1 human melanoma antigen recognized by the majority of HLA-A2-restricted tumor infiltrating lymphocytes. J. Exp. Med. 180:347-352.
55. Kilby, J. M., Hopkins S., Venetta T. M., DiMassimo B., Cloud G. A., Lee J. Y., Alldredge L., Hunter E., Lambert D., Bolognesi D., Matthews T., Johnson M. R., Nowak M. A., Shaw G. M. and Saag M. S. (1998) Potent suppression of HIV-1 replication in humans by T-20, a peptide inhibitor of gp41-mediated virus entry. Nat. Med. 4:1302-7.
56. Klasse, P. J., McKeating J. A., Schutten M., Reitz M. S. Jr. and Robert-Guroff M. (1993) An immune-selected point mutation in the transmembrane protein of human immunodeficiency virus type 1 (HXB2-Env:Ala 582(-->Thr)) decreases viral neutralization by monoclonal antibodies to the CD4-binding site. Virology 196:332-337.
57. Klinman, D. M., Yamshchikov G and Ishigatsubo Y. (1997) Contribution of CpG motifs to the immunogenicity of DNA vaccines. J. Immunol. 158:3635-3639.
58. Kovacsovics-Bankowski, M. and Rock K. L. (1995) A phagosome-to-cytosol pathway for exogenous antigens presented on MHC class I molecules. Science 267:243-246.
59. Kwon, B. S., Haq A. K., Pomerantz S. H. and Halaban R. (1997) Isolation and sequence of a cDNA clone for human tyrosinase that maps at the mouse c-albino locus. Proc. Natl. Acad. Sci. U.S.A. 84:7473-7477.
60. Kwong, P. D., Wyatt R., Sattentau Q. J., Sodroski J. and Hendrickson W. A. (2000) Oligomeric modeling and electrostatic analysis of the gp120 envelope glycoprotein of human immunodeficiency virus. J. Virol. 74:1961-1972.
61. LaCasse, R. A., Follis K. E., Trahey M., Scarborough J. D., Littman D. R. and Nunberg J. H. (1999) Fusion-competent vaccines: broad neutralization of primary isolates of HIV. Science 283:357-362.
62. Lambin, P., Rochu D., Herance N. and Fine J. M. (1982) High molecular-weight polymers in human albumin solutions. Quantitative determination by polyacrylamide gradient electrophoresis Rev. Fr. Transfus. Immunohematol. 25:487-498. French.
63. Langhein, C. and Newman J. F. (1987) Antibody response to bacterial antigens covalently bound to biodegradable polymerized serum albumin beads. Journal of Applied Bacteriology 63:443-448.
64. Lewis, J. K., Bendahmane M., Smith T. J., Beachy R. N. and Siuzdak G. (1998) Identification of viral mutants by mass spectrometry. Proc. Nat. Acad. Sci. U.S.A. 95:8596-8601.
65. Lu et al, (1998) AIDS Res. Human Retrovir. 14:151-155.
66. Mascola, J. R., Snyder S. W., Weislow O. S., Belay S. M., Belshe R. B., Schwartz D. H., Clements M. L., Dolin R., Graham B. S, Gorse G. J., Keefer M. C., McElrath M. J., Walker M. C., Wagner K. F., McNeil J. G., McCutchan F. E. and Burke D. S. (1996) Immunization with envelope subunit vaccine products elicits neutralizing antibodies against laboratory-adapted but not primary isolates of human immunodeficiency virus type 1. The National Institute of Allergy and Infectious Diseases AIDS Vaccine Evaluation Group. Infec. Dis. 173:340-348.
67. Men, Y., Gander B., Merkle H. P., and Corradin G. (1996) Induction of sustained and elevated immune responses to weakly immunogenic synthetic malarial peptides by encapsulation in biodegradable polymer microspheres. Vaccine 14:1442-1450.
68. Miyahira, Y., Murata K., Rodriguez D., Rodriguez J. R., Esteban M., Rodrigues M. M. and Zavala F. (1995) Quantification of antigen specific CD8+ T cells using an ELISPOT assay. J. Immunol. Methods 181:45-54.
69. Moore, J. P. and Sodroski J. (1996) Antibody cross-competition analysis of the human immunodeficiency virus type 1 gp120 exterior envelope glycoprotein. J. Virol. 70: 1863-1872.
70. Moore, J. P., Sattentau Q. J., Wyatt R. and Sodroski J. (1994a) Probing the structure of the human immunodeficiency virus surface glycoprotein gp120 with a panel of monoclonal antibodies. J. Virol 68:469-484.
71. Moore, J. P., Trkola A., Korber B., Boots L. J., Kessler J. A. 2nd, McCutchan F. E., Mascola J., Ho D. D., Robinson J. and Conley A. J. (995) HIV-1 neutralization: the consequences of viral adaptation to growth on transformed T cells. AIDS. 9 [Suppl A]: S117-S136.
72. Moore, J. P., Trkola A., Korber B., Boots L. J., Kessler J. A. 2nd, McCutchan F. E., Mascola J., Ho D. D., Robinson J. and Conley A. J. (1995a) A human monoclonal antibody to a complex epitope in the V3 region of gp120 of human immunodeficiency virus type 1 has broad reactivity within and outside clade B. J. Virol. 69:122-130.
73. Moore, J. P., Willey R. L., Lewis G. K., Robinson J., and Sodroski J. (1994b) Immunological evidence for interactions between the first, second, and fifth conserved domains of the gp120 surface glycoprotein of human immunodeficiency virus type 1. J. Virol. 68:6836-6847.
74. Muster, et al. (1994) Cross-neutralizing activity against divergent human immunodeficiency virus type 1 isolates induced by the gp41 sequence ELDKWAS. J. Virol. 68:4031-4034.
75. Muster, T., Steindl F., Purtscher M., Trkola A., Klima A., Himmler G., Ruker F. and Katinger H. (1993) A conserved neutralizing epitope on gp41 of human immunodeficiency virus type 1. J. Virol. 67:6642-6647.
76. Myszka, D. G., Sweet R. W., Hensley P., Brigham-Burke M., Kwong P. D., Hendrickson W. A., Wyatt R., Sodroski J. and Doyle M. L. (2000) Energetics of the HIV gp120-CD4 binding reaction. Proc. Natl. Acad. Sci. U.S.A. 97:9026-9031.
77. O'Brien, W. A., Koyanagi, Namazie A., Zhao J. Q., Diagne A., Idler K., Zack J. A. and Chen I. S. (1990) HIV-1 tropism for mononuclear phagocytes can be determined by regions of gp120 outside the CD4-binding domain. Nature 348: 69-73.
78. O'Hagan, D. T., Jeffery H. and Davis S. S. (1993) Long-term antibody responses in mice following subcutaneous immunization with ovalbumin entrapped in biodegradable microparticles. Vaccine 11:965-969.
79. Okayama, H. and Berg P. (1983) A cDNA cloning vector that permits expression of cDNA inserts in mammalian cells. Mol. Cell. Biol. 3(2):280-289.
80. Olson, W. C., Rabut G. E., Nagashima K. A., Tran D. N., Anselma D. J., Monard S. P., Segal J. P., Thompson D. A., Kajumo F., Guo Y., Moore J. P., Maddon P. J. and Dragic T. (1999) Differential inhibition of human immunodeficiency virus type 1 fusion, gp120 binding, and CC-chemokine activity by monoclonal antibodies to CCR5. J. Virol. 73:4145-4155.
81. Overwijk, W. W., Tsung A., Irvine K. R., Parkhurst M. R., Goletz T. J., Tsung K., Carroll M. W., Liu C., Moss B., Rosenberg S. A., and Restifo N. P. (1998) Gp100/pmel 17 is a murine tumor rejection antigen: induction of “self”-reactive, tumoricidal T cells using high-affinity, altered peptide ligand. J. Exp. Med. Vol 188(2):277-286.
82. Owens, J. M., Matsuo K., Nicholson G. C., Wagner E. F. and Chambers T. J. (1999) Fra-1 potentiates osteoclastic differentiation in osteoclast-macrophage precursor cell lines. Virology 179:827-833.
83. Parker, C. E., Deterding L. J., Hager-Braun C., Binley J. M., Schulke N., Katinger H., Moore J. P. and Tomer K. B. (2001) Fine definition of the epitope on the gp41 glycoprotein of human immunodeficiency virus type 1 for the neutralizing monoclonal antibody 2F5. J. Virol. 75:10906-10911.
84. Parren P. W., Mondor I., Naniche D., Ditzel H. J., Klasse P. J., Burton D. R. and Sattentau Q. J. (1998) Neutralization of human immunodeficiency virus type 1 by antibody to gp120 is determined primarily by occupancy of sites on the virion irrespective of epitope specificity. J. Virol 72:3512-3519.
85. Parren, P. W., Fisicaro P., Labrijn A. F., Binley J. M., Yang W. P., Ditzel H. J., Barbas C. F. 3^{rd} and Burton D. R. (1996) In vitro antigen challenge of human antibody libraries for vaccine evaluation: the human immunodeficiency virus type 1 envelope. J. Virol. 70:9046-9050.
86. Parren, P. W., Moore J. P., Burton D. R. and Sattentau Q. J. (1999) The neutralizing antibody response to HIV-1: viral evasion and escape from humoral immunity. AIDS 13 Suppl. A:S137-S162.
87. Partidos, C. D., Vohra P., Anagnostopoulou C., Jones D. H., Farrar G. H. and Steward M. W. (1996) Biodegradable microparticles as a delivery system for measles virus cytotoxic T cell epitopes. Mol. Immunol. 33:485-491.
88. Pertmer, T. M., Roberts T. R. and Haynes J. R. (1996) Influenza virus nucleoprotein-specific immunoglobulin G subclass and cytokine responses elicited by DNA vaccination are dependent on the route of vector DNA delivery. J. Virol. 70(9):6119-6125.
89. Powell, M. F., Cleland J. L., Eastman D. J., Lim A., Murthy K., Newman M. J., Nunberg J. H., Weissburg R. P., Vennari J. C. and Wrin T. (1994) Immunogenicity and HIV-1 virus neutralization of MN recombinant glycoprotein 120/HIV-1 QS21 vaccine in baboons [published erratum appears in AIDS Res Hum Retroviruses 1995 11(5):661]. AIDS Res. Hum. Retroviruses 10 Suppl 2:S105-8:S105-S108.
90. Prayaga, S. K., Ford M. J. and Haynes J. R. (1997) Manipulation of HIV-1 gp120-specific immune responses elicited via gene gun-based DNA immunization. Vaccine. 15(12-13):1349-52.
91. Raychaudhuri, S. and Rock K. L. (1998) Fully mobilizing host defense: building better vaccines. Nat. Biotechnol. 16:1025-1031.
92. Raz, E., Tighe H., Sato Y., Corr M., Dudler J. A., Roman M., Swain S. L., Spiegelberg H. L. and Carson D. A. (1996) Preferential induction of a Th1 immune response and inhibition of specific IgE antibody formation by plasmid DNA immunization. Proc. Natl. Acad. Sci. U.S.A. 93(10):5141-5145.
93. Reitter, J. N., Means R. E. and Desrosiers R. C. (1998) A role for carbohydrates in immune evasion in AIDS. Nat. Med. 4:679-684.
94. Reitz, M. S. Jr, Wilson C., Naugle C., Gallo R. C. and Robert-Guroff M. (1988) Generation of a neutralization-resistant variant of HIV-1 is due to selection for a point mutation in the envelope gene. Cell 54:57-63.
95. Robinson, H. L. (1997) Nucleic acid vaccines: an overview. Vaccine. 15(8):785-787.
96. Rock, K. L. and Clark K. (1996) Analysis of the role of MHC class II presentation in the stimulation of cytotoxic T lymphocytes by antigens targeted into the exogenous antigen-MHC class I presentation pathway. J. Immunol. 156:3721-3726.
97. Roux, K. H. (1989) Immunoelectron microscopy of idiotype-anti-idiotype complexes. Methods Enzymol. 178:130-144.
98. Roux, K. H. (1996) Negative-Stain Immunoelectron-Microscopic Analysis of Small Macromolecules of Immunologic Significance Methods 10:247-256.
99. Saphire, E. O., Parren P. W., Pantophlet R., Zwick M. B., Morris G. M., Rudd P. M., Dwek R. A., Stanfield R. L., Burton D. R. and Wilson I. A. (2001) Crystal structure of a neutralizing human IGG against HIV-1: a template for vaccine design. Science 293:1155-1159.
100. Sattentau, Q. J. and Moore J. P. (1991) Conformational changes induced in the human immunodeficiency virus envelope glycoprotein by soluble CD4 binding. J. Exp. Med. 174:407-415.
101. Sattentau, Q. J., Zolla-Pazner S. and Poignard P. (1995) Epitope exposure on functional, oligomeric HIV-1 gp41 molecules. Virology 206:713-717.
102. Scandella, C. J., Kilpatrick J., Lidster W., Parker C., Moore J. P., Moore G. K., Mann K. A., Brown P., Coates S., Chapman B., et al. (1993) Nonaffinity purification of recombinant gp120 for use in AIDS vaccine development. AIDS Res. Hum. Retroviruses 9:1233-1244.
103. Schägger, H., Cramer W. A. and von Jagow G. (1994) Analysis of molecular masses and oligomeric states of protein complexes by blue native electrophoresis and isolation of membrane protein complexes by two-dimensional native electrophoresis. Analytical Biochemistry 217:220-230.
104. Schulz, T. F., Jameson B. A., Lopalco L., Siccardi A. G., Weiss R. A. and Moore J. P. (1992) Conserved structural features in the interaction between retroviral surface and transmembrane glycoproteins? AIDS Res. Human Retrovir. 8: 1571-1580.
105. Shibata, R., Hoggan M. D., Broscius C., Englund G., Theodore T. S., Buckler-White A., Arthur L. O., Israel Z., Schultz A., Lane H. C., et al. (1995) Isolation and characterization of a syncytium-inducing, macrophage/T-cell line-tropic human immunodeficiency virus type 1 isolate that readily infects chimpanzee cells in vitro and in vivo. J. Virol 69:4453-4462.
106. Sjolander, A., van't Land B. and Lovgren Bengtsson K. (1997). Iscoms containing purified Quillaja saponins upregulate both Th1-like and Th2-like immune responses. Cell. Immunol. 177(1):69-76.
107. St. Clair, N., Shenoy B., Jacob L. D. and Margolin A. L. (1999) Cross-linked protein crystals for vaccine delivery. Proc. Nat. Acad. Sci. U.S.A. 96:9469-9474.
108. Sullivan, N., Sun Y., Sattentau Q., Thali M., Wu D., Denisova G., Gershoni J., Robinson J., Moore J. and Sodroski J. (1998) CD4-Induced conformational changes in the human immunodeficiency virus type 1 gp120 glycoprotein: consequences for virus entry and neutralization. J. Virol. 72:4694-4703.
109. Takeuchi, Y., Inagaki M., Kobayashi N. and Hoshino H. (1987) Isolation of human immunodeficiency virus from a Japanese hemophilia B patient with AIDS. Jpn. J. Cancer Res. 78:11-15.
110. Tan, K., Liu J., Wang J., Shen S. and Lu M. (1997) Atomic structure of a thermostable subdomain of HIV-1 gp41. Proc. Natl. Acad. Sci. U.S.A. 94:12303-12308.
111. Thali, M., Moore J. P., Furman C., Charles M., Ho D. D., Robinson J. and Sodroski J. (1993) Characterization of conserved human immunodeficiency virus type 1 gp120 neutralization epitopes exposed upon gp120-CD4 binding. J. Virol. 67:3978-3988.
112. Thomas, G., Thorne B. A., Thomas L., Allen R. G., Hruby D. E., Fuller R. and Thorner J. (1988) Yeast KEX2 endopeptidase correctly cleaves a neuroendocrine prohormone in mammalian cells. Science 241:226-230.
113. Trkola, A., Dragic T., Arthos J., Binley J. M., Olson W. C., Allaway G. P., Cheng Mayer C., Robinson J., Maddon P. J., and Moore J. P. (1996a) CD4-dependent, antibody-sensitive interactions between HIV-1 and its co-receptor CCR-5. Nature 384:184-187.
114. Trkola, A., Ketas T., Kewalramani V. N., Endorf F., Binley J. M., Katinger H., Robinson J., Littman D. R. and Moore J. P. (199⁸)-Neutralization sensitivity of human immunodeficiency virus type 1 primary isolates to antibodies and CD4-based reagents is independent of coreceptor usage. J. Virol. 72:1876-1885.
115. Trkola, A., Pomales A. B., Yuan H., Korber B., Maddon P. J., Allaway G. P., Katinger H., Barbas C. F. 3rd, Burton D. R., Ho D. D., et al. (1995) Cross-clade neutralization of primary isolates of human immunodeficiency virus type 1 by human monoclonal antibodies and tetrameric CD4-IgG. J. Virol. 69:6609-6617.
116. VanCott, T. C., Mascola J. R., Kaminski R. W., Kalyanaraman V., Hallberg P. L., Burnett P. R., Ulrich J. T., Rechtman D. J. and Birx D. L. (1997) Antibodies with specificity to native gp120 and neutralization activity against primary human immunodeficiency virus type 1 isolates elicited by immunization with oligomeric gp160. J. Virol. 71:4319-4330.
117. Venkatesh, S. G. and Deshpande V. (1999) A comparative review of the structure and biosynthesis of thyroglobulin. Comp. Biochem. Physiol. C. Pharmacol. Toxicol. Endocrinol. 122:13-20.
118. Vidard, L., Kovacsovics-Bankowski M., Kraeft S. K., Chen L. B., Benacerraf B., and Rock K. L. (1996) Analysis of MHC class II presentation of particulate antigens of B lymphocytes. J. Immunol. 156:2809-2818
119. White-Scharf, M. E., Potts B. J., Smith L. M., Sokolowski K. A., Rusche J. R. and Silver S. (1993) Broadly neutralizing monoclonal antibodies to the V3 region of HIV-1 can be elicited by peptide immunization. Virology 192:197-206.
120. Wu, L., Gerard N. P., Wyatt R., Choe H., Parolin C., Ruffing N., Borsetti A., Cardoso A. A., Desjardin E., Newman W., Gerard C. and Sodroski J. (1996) CD4-induced interaction of primary HIV-1 gp120 glycoproteins with the chemokine receptor CCR-5. Nature 384:179-183.
121. Wyatt, R. and Sodroski J. (1998b) The HIV-1 envelope glycoproteins: fusogens, antigens, and immunogens. Science 280:1884-1888.
122. Wyatt, R., Desjardin E., Olshevsky U., Nixon C., Binley J., Olshevsky V. and Sodroski J. (1997) Analysis of the interaction of the human immunodeficiency virus type 1 gp120 envelope glycoprotein with the gp41 transmembrane glycoprotein. J. Virol. 71:9722-9731.
123. Wyatt, R., Kwong P. D., Desjardins E., Sweet R. W., Robinson J., Hendrickson W. A. and Sodroski J. G. (1998a) The antigenic structure of the HIV gp120 envelope glycoprotein. Nature 393:705-711.
124. Wyatt, R., Moore J., Accola M., Desjardin E., Robinson J., Sodroski J. (1995) Involvement of the V1/V2 variable loop structure in the exposure of human immunodeficiency virus type 1 gp120 epitopes induced by receptor binding. J. Virol. 69:5723-5733.
125. Zhang, C. W., Chishti Y., Hussey R. E. and Reinherz E. L. (2001) Expression, purification, and characterization of recombinant HIV gp140. The gp41 ectodomain of HIV or simian immunodeficiency virus is sufficient to maintain the retroviral envelope glycoprotein as a trimer. J. Biol. Chem. 276:39577-39585.
126. Zhang, W., Canziani G., Plugariu C., Wyatt R., Sodroski J., Sweet R., Kwong P., Hendrickson W. and Chaiken I. (1999) Conformational changes of gp120 in epitopes near the CCR5 binding site are induced by CD4 and a CD4 miniprotein mimetic. Biochemistry 38:9405-9416.
127. Zhu, P., Olson W. C. and Roux K. H. (2001) Structural flexibility, and functional valence of CD4-IgG2 (PRO 542): potential for cross-linking human immunodeficiency virus type 1 envelope spikes. J. Virol. 75:6682-6686.

1. A composition comprising a pharmaceutically acceptable particle and a stable HIV-1 pre-fusion envelope glycoprotein trimeric complex operably affixed thereto, each monomeric unit of the complex comprising HIV-1 gp120 and HIV-1 gp41, wherein (i) the gp120 and gp41 are bound to each other by at least one disulfide bond between a cysteine residue introduced into the gp120 and a cysteine residue introduced into the gp41, and (ii) the gp120 has deleted from it at least one V-loop present in wild-type HIV-1 gp120.

2. The composition of claim 1, wherein the stable HIV-1 pre-fusion envelope glycoprotein trimeric complex is operably affixed to the particle via an agent which is operably affixed to the particle.

3. The composition of claim 1, further comprising a pharmaceutically acceptable carrier.

4. The composition of claim 1, further comprising an adjuvant.

5. The composition of Claim 1, wherein the gp120 has deleted from it one or more of variable loops V1, V2 and V3.

6. The composition of claim 1, wherein the disulfide bond is formed between a cysteine residue introduced by an A492C mutation in gp120 and a cysteine residue introduced by a T596C mutation in gp41.

7. The composition of claim 1, wherein the gp120 is further characterized by (i) the absence of one or more canonical glycosylation sites present in wild-type HIV-1 gp120, and/or (ii) the presence of one or more canonical glycosylation sites absent in wild-type HIV-1 gp120.

8. The composition of claim 1, wherein the particle is selected from the group consisting of a paramagnetic bead, a non-paramagnetic bead, a liposome and any combination thereof.

9. The composition of claim 1, wherein the particle comprises PLG, latex, polystyrene, polymethyl-methacrylate, or any combination thereof.

10. The composition of claim 1, wherein the mean diameter of the particle is from about 10 nm to 100 μm.

11. The composition of claim 10, wherein the mean diameter of the particle is from about 100 nm to 10 μm.

12. The composition of claim 10, wherein the mean diameter of the particle is from about 100 nm to 1 μm.

13. The composition of claim 10, wherein the mean diameter of the particle is from about 1 μm to 10 μm.

14. The composition of claim 10, wherein the mean diameter of the particle is from about 10 μm to 100 μm.

15. The composition of claim 1, wherein the mean diameter of the particle is from about 10 nm to 100 nm.

16. The composition of claim 1, wherein the mean diameter of the particle is about 50 nm.

17. The composition of claim 2, wherein the agent is selected from the group consisting of an antibody, a fusion protein, streptavidin, avidin, a lectin, and a receptor.

18. The composition of claim 2, wherein the agent is CD4.

19. The composition of claim 17, wherein the agent is an antibody.

20. The composition of claim 4, wherein the adjuvant is selected from the group consisting of alum, Freund's incomplete adjuvant, saponin, Quil A, QS-21, Ribi Detox, monophosphoryl lipid A, a CpG oligonucleotide, CRL-1005, L-121, and any combination thereof.

21. The composition of claim 3, further comprising a cytokine and/or a chemokine.

22. The composition of claim 21, wherein the cytokine is selected from the group consisting of interleukin-2, interleukin-4, interleukin-5, interleukin-12, interleukin-15, interleukin-18, GM-CSF, and any combination thereof.

23. The composition of claim 21, wherein the chemokine is selected from the group consisting of SLC, ELC, Mip3α, Mip3β, IP-10, MIG, and any combination thereof.

24. A method for eliciting an immune response in a subject against HIV-1 or an HIV-1-infected cell comprising administering to the subject a prophylactically or therapeutically effective amount of the composition of claim 1.

25-27. (canceled)

28. A vaccine which comprises a therapeutically effective amount of the composition of claim 1 and a pharmaceutically acceptable carrier.

29. A vaccine which comprises a prophylactically effective amount of the composition of claim 1 and a pharmaceutically acceptable carrier.

30. A method for preventing a subject from becoming infected with HIV-1 comprising administering to the subject a prophylactically effective amount of the composition of claim 1, thereby preventing the subject from becoming infected with HIV-1.

31. A method for reducing the likelihood of a subject's becoming infected with HIV-1 comprising administering to the subject a prophylactically effective amount of the composition of claim 1, thereby reducing the likelihood of the subject's becoming infected with HIV-1.

32-33. (canceled)

34. A method for producing the composition of claim 1, comprising contacting a pharmaceutically acceptable particle with a stable HIV-1 pre-fusion envelope glycoprotein trimeric complex under conditions permitting the complex to become operably affixed to the particle, wherein each monomeric unit of the complex comprises HIV-1 gp120 and HIV-1 gp41, (i) the gp120 and gp41 being bound to each other by at least one disulfide bond between a cysteine residue introduced into the gp120 and a cysteine residue introduced into the gp41, and (ii) the gp120 having deleted from it at least one V-loop present in wild-type HIV-1 gp120.

35-73. (canceled)
